(19) United States
(12) Patent Application Publication
(10) Pub. No.: US 2021/0002706 A1
(43) Pub. Date: Jan. 7, 2021

(54) Methods For Determining Antimicrobial Drug Resistance

(71) Applicant: Ares Genetics GmbH, Vienna (AT)

(72) Inventors: Norhan MAHFOUZ, Vienna (AT); Stephan BEISKEN, Vienna (AT); Andreas POSCH, Vienna (AT)

(21) Appl. No.: 16/920,109

(22) Filed: Jul. 2, 2020

(30) Foreign Application Priority Data
Jul. 5, 2019 (EP) 19 184 836.5

(51) Int. Cl. C12Q 1/689; G16B 20/00; G16B 50/20

(52) U.S. Cl. CPC C12Q 1/689; G16B 20/00; C12Q 2600/158; C12Q 2600/106; C12Q 2600/156; G16B 50/20

(57) ABSTRACT

The present invention provides for the prediction or detection of antimicrobial drug resistance in a microorganism, particularly in a pathogenic microorganism, by determining the presence or absence of antimicrobial resistance markers (AMRs) in the microorganism. This allows for the rapid determination of whether a microorganism is resistant to one or more antimicrobial drugs. The microorganism can be obtained from a patient suffering or suspected of suffering from an infection by the microorganism, and once the determination is made, the patient can be given an antimicrobial drug to which the microorganism is not resistant.

### FIELD OF THE INVENTION

The invention disclosed herein relates to methods for the detection or prediction of the ability of a microorganism to be resistant to one or more antibiotic compounds or drugs, i.e., the ability to still be able to grow in the presence of such antibiotic. The methods are particularly useful in determining whether a microorganism, causing or suspected of causing an infection in a patient, is resistant to a particular antibiotic, so that the patient can be treated with an antibiotic against which the microorganism is not known to be resistant.

### BACKGROUND OF THE INVENTION

Antimicrobial drug resistance is on the rise globally. Estimates are that 700,000 to several million deaths result per year from infections caused by antimicrobial drug resistant microorganisms. For example, each year in the U.S., at least 2 million people become infected with bacteria that are resistant to antibiotics and at least 23.000 people die as a result. For instance, the so-called “ESKAPE” pathogens, e.g., Enterococcus faecium, Staphylococcus aureus, Klebsiella pneumoniae, Acinetobacter baumanii, Pseudomonas aeruginosa, and Enterobacter species, are known to be major causes of hospital acquired infections worldwide.

Rising antimicrobial drug/antibiotic resistance is caused mainly by their overuse in humans and animals, spread of resistant strains between the two, and dumping of inadequately treated effluents from industry. Antibiotics are known to increase selective pressure in bacterial populations, causing susceptible bacteria to die. This increases the percentage of resistant bacteria which continue growing. Even at very low dosage levels of administered antibiotics, resistant bacteria may have a growth advantage over susceptible bacteria. With resistance to antibiotics becoming more common there is greater need for alternative way to quickly identify resistant microorganisms such that patients can be administered effective antimicrobials and avoid administering antimicrobials to which the microorganisms are resistant. This leads not only to faster recovery but also avoids any unnecessary selective pressure on the microorganisms not causing the infection.

It is known that antibiotic resistance can be associated with genetic polymorphisms. For example, it has been shown that resistance can be correlated with genetic changes in the bacteria. Wozniak et al., 2012, BMC Genomics 13:S23 disclosed genetic determinants of drug resistance in Staphylococcus aureus based on genotype and phenotype data. Stoesser et al., 2013, J Antimicrob Chemother 68: 2234-2244 disclosed prediction of antimicrobial susceptibilities for Escherichia coli and Klebsiella pneumoniae isolates using whole genomic sequence data. Chewapreecha et al., 2014, PLoS Genet 10(8):e1004547 described a comprehensive identification method of single nucleotide polymorphisms associated with beta-lactam resistance within pneumococcal mosaic genes.

Such antimicrobial drug/antibiotic resistance mechanisms that emerge and spread globally challenge medical personnel's ability to treat common bacterial infections by reducing the effectiveness of or completely nullifying the effects of existing treatment methodologies. This inevitably leads to increased healthcare expenditure, increased length of stay at healthcare institution, and likely leads to a higher mortality rate. Specifically, antibiotic resistance not only makes diagnosing existing bacterial infections difficult, but it also has subsequent effects in other fields of healthcare.

Existing techniques for combating antibiotic resistance generally involve preventing the spread of antibiotic resistant microorganisms including strategies for controlling antibiotics consumption at the policy and management level, rather than predicting or detecting the various genetic causes of a resistance or a multiple resistance in a microorganism. Typically, a microorganism suspected of being resistant is tested for one or more known specific mutations in known resistance genes. However, these techniques are time- and labor-intensive. There is a need in the art for methods which can quickly determine whether an infectious microorganism is resistant to one or more antimicrobial drugs such that the patient can be administered, in a timelier manner, an antimicrobial drug to which the microorganism causing the infection is susceptible.

The present invention addresses the needs in the art by providing methods and antimicrobial resistance markers (AMRs), which can be used to predict or detect antimicrobial drug resistance/susceptibility of a microorganism, such as a bacterium causing or suspected of causing an infection in a patient.

### SUMMARY OF THE INVENTION

Disclosed herein are methods for determining or predicting the ability of a microorganism to be resistant to one or more antibiotic compounds or to one or more different classes of antibiotic compounds. In other words, the disclosed methods can predict or detect the antimicrobial drug resistance status of a microorganism, i.e., whether the microorganism is resistant or not resistant to one or more particular antibiotic compounds, which compounds can be from one or more different classes of antibiotic compounds. These methods can be used to diagnose infections caused by resistant microorganisms and allow for faster effective treatment of a patient by providing an antibiotic to which the microorganism is not resistant to a patient suffering from such an infection.

The present invention is based, in part, on the identification of antimicrobial resistance markers (AMRs), which when present or absent in a microorganism are indicative of not only the microorganism in which the marker and its correlation to resistance was originally observed but also in microorganisms of other genera, species or strains. In certain cases, the presence or absence of the AMR is directly responsible for the phenotypic change in resistance, in other cases, the presence or absence is statistically associated with such resistance.

The present invention is directed to a method for determining or predicting the antimicrobial drug resistance status of a (any) microorganism, which method comprises:
(i) determining the presence or absence in the microorganism of at least one nucleotide sequence identified in Table 1 or a variant of the nucleotide sequence, and/or
(ii) determining the presence or absence in the microorganism of at least one difference in at least one nucleotide sequence identified in Table I compared with a reference sequence,
wherein the presence or absence of the at least one nucleotide sequence and/or difference indicates the antimicrobial drug resistance status of the microorganism.

In an embodiment, one type of antimicrobial resistance marker (AMR) is a nucleotide sequence identified or a variant of the nucleotide sequence. Another type of AMR is a difference in a nucleotide sequence compared with a reference sequence.

In an embodiment, the microorganism being subject to the method for determining or predicting the antimicrobial drug resistance status of a microorganism is a microorganism different from the microorganism identified in Table I associated with the respective at least one nucleotide sequence and/or the at least one difference in at least one nucleotide sequence (AMR or mkr_id), the presence or absence of which is indicative of antimicrobial drug resistance of the microorganism being subject to the method. As used herein, a different microorganism is a microorganism of a different strain or subspecies, of a different species or of a different genus. Herein, the terms AMR, mkr_id or marker are used interchangeably.

In an embodiment, the at least one nucleotide sequence and/or the at least one difference in at least one nucleotide sequence (AMR) is identified in Table I in rows 0 to 3840. In an embodiment, the AMR is identified in Table I in rows 0 to 3751. In an embodiment, the AMR used in the methods of the invention is not one in rows 3752 to 3840 identified in Table 1. These AMRs (identified in rows 3752 to 3840 of Table I) are known for being indicative of antibiotic resistance in the microorganism in which they were first determined (which microorganism is given in Table I for each respective AMR). However, prior to the present invention as disclosed herein, these AMRs were not known to be indicative of antibiotic resistance when present or absent in microorganisms of different strains, species or genera, and along with all the other AMRs listed in Table I are useful in determining or predicting the antimicrobial drug resistance of any microorganism in which the AMR is accordingly present or absent. The column in Table I entitled Marker associated with Cross-Resistance indicates with a + those AMRs that when present or absent are indicative of resistance to more than one class/type of antibiotic drug. The column in Table I entitled Associated with resistance in multiple pathogens indicates with a + those AMRs that when present or absent are indicative of resistance in more than one kind of microorganism. The column in Table I entitled cross-resistance in multiple pathogens indicates with a + those AMRS that when present or absent are indicative of resistance to more than one class/type of antibiotic drug in more than one kind of microorganism.

In an embodiment, the presence or absence of the at least one nucleotide sequence and/or difference indicates that the microorganism is resistant to one or more antimicrobial drugs, preferably to one or more classes of antimicrobial drugs (e.g., to at least two different types of antimicrobial drugs). In an embodiment, the presence or absence of at least two nucleotide sequences in step (i) is determined or wherein the presence or absence of at least two differences in step (ii) is determined. In an embodiment, more than one difference in one nucleotide sequence in step (ii) is determined. In an embodiment, the AMR is associated with resistance to more than one antimicrobial drug. In an embodiment, the AMR is associated with resistance to more than one class of antimicrobial drugs. In an embodiment, the AMR is associated with resistance in more than one type of microorganism. In an embodiment, the AMR is associated with resistance to more than one antimicrobial drug in more than one type of microorganism. In an embodiment, the AMR is associated with resistance to more than one class of antimicrobial drugs in more than one type of microorganism.

In an embodiment, the difference in the nucleotide sequence is at the position (nc_pos) in the nucleotide sequence identified in Table I or the difference at the position in the nucleotide sequence is the nc_alt residue for that nucleotide sequence identified in Table 1 or the difference in the nucleotide sequence is a point mutation resulting in a change in the encoded amino acid sequence or which change results in a stop codon.

In certain embodiments, the AMR, the presence or absence of which in step (i) and/or (ii) is determined, is identified in rows 0 to 3840 of Table I, preferably in rows 0 to 3751 of Table I.

In an embodiment, the AMR, the presence or absence of which in step (i) and/or (ii) is determined, identified in the following rows of Table I is excluded in the methods of the invention: 3752 to 3840. In certain embodiments, when the microorganism, whose antimicrobial drug resistance status is to be determined or predicted, is Acinetobacter baumannii, the AMRs identified in rows 3752 to 3764 are excluded; when the microorganism is Escherichia coli, the AMRs identified in rows 3778 to 3784 are excluded; when the microorganism is Enterobacter cloacae, the AMRs identified in rows 3768 to 3776 are excluded; when the microorganism is Klebsiella aerogenes, the AMRs identified in rows 3785 to 3787 are excluded; when the microorganism is Klebsiella pneumoniae, the AMRs identified in rows 3788 to 3797 are excluded; when the microorganism is Pseudomonas aeruginosa, the AMRs identified in rows 3008 and 3009 are excluded; when the microorganism is Morganella morganii, the AMR identified in row 3798 is excluded; when the microorganism is Salmonella enterica, the AMRs identified in rows 3810 to 3815 are excluded; when the microorganism is Proteus mirabilis, the AMRs identified in rows 3799 to 3807 are excluded; when the microorganism is Serratia mascescens, the AMR identified in row 3816 is excluded; when the microorganism is Stenotrophomonas maltophilia, the AMR identified in row 3840 is excluded; when the microorganism is Shigella boydii, the AMRs identified in rows 3817 to 3820 are excluded; when the microorganism is Citrobacter freundii, the AMRs identified in rows 3765 to 3766 is excluded; when the microorganism is Shigella sonnei, the AMRs identified in rows 3821 to 3823 are excluded; when the microorganism is Staphylococcus aureus, the AMRs identified in rows 3824 to 3839 are excluded; when the microorganism is Citrobacter koseri, the AMR identified in row 3767 is excluded; when the microorganism is Enterobacter roggenkampii, the AMR identified in row 3777 is excluded.

The microorganism whose resistance is to be determined/predicted can be selected from the group consisting of Achromobacter, Acinetobacter, Burkholderia, Citrobacter, Enterobacter, Escherichia, Klebsiella, Kluyvera, Lelliottia, Morganella, Pantoea, Pluralibacter, Proteus, Providencia, Pseudomonas, Raoultella, Salmonella, Serratia, Shigella, Staphylococcus, and Stenotrophomonas spp.

Exemplary microorganisms include those listed in Table II. Particular microorganisms can include Achromobacter xylosoxidans, Acinetobacter baumannii, Acinetobacter beijerinckii, Acinetobacter bereziniae, Acinetobacter calcoaceticus, Acinetobacter gyllenbergii, Acinetobacter haemolyticus, Acinetobacter indicus, Acinetobacter johnsonii, Acinetobacter junii, Acinetobacter lactucae, Acinetobacter lwoffii, Acinetobacter nosocomialis, Acinetobacter oleivorans, Acinetobacter pittii, Acinetobacter radioresistens, Acinetobacter schindleri, Acinetobacter soli, Acinetobacter sp. ADP1, Acinetobacter sp. AR_0276, Acinetobacter sp. WC-743, Acinetobacter tandoii, Acinetobacter ursingii, Acinetobacter venetianus, Burkholderia ambifaria, Burkholderia cenocepacia, Citrobacter amalonaticus, Citrobacter braakii, Citrobacter freundii, Citrobacter koseri, Citrobacter pasteurii, Citrobacter portucalensis, Citrobacter rodentium, Citrobacter werkmanii, Enterobacter asburiae, Enterobacter bugandensis, Enterobacter cancerogenus, Enterobacter cloacae, Enterobacter cloacae complex, Enterobacter cloacae complex sp. FDA-CDC-AR_0132, Enterobacter cloacae complex sp. FDA-CDC-AR_0164, Enterobacter hormaechei, Enterobacter kobei, Enterobacter ludwigii, Enterobacter roggenkampii, Enterobacter sp. 638, Enterobacter sp. BIDMC 28, Enterobacter sp. MGH 1, Enterobacter sp. MGH 10, Enterobacter sp. MGH 14, Enterobacter sp. MGH 15, Enterobacter sp. MGH 22, Enterobacter sp. MGH 23, Enterobacter sp. MGH 24, Enterobacter sp. MGH 25, Enterobacter sp. MGH 3, Enterobacter sp. MGH 33, Enterobacter sp. MGH 37, Enterobacter sp. MGH 38, Enterobacter sp. MGH 6, Enterobacter sp. MGH 7, Enterobacter sp. R4-368, Enterococcus faecalis, Escherichia albertii, Escherichia coli, Klebsiella aerogenes, Klebsiella grimontii, Klebsiella michiganensis, Klebsiella oxytoca, Klebsiella pneumoniae, Klebsiella quasipneumoniae, Klebsiella variicola, Kluyvera cryocrescens, Kluyvera intermedia, Lelliottia amnigena, Morganella morganii, Pantoea agglomerans, Pantoea vagans, Pluralibacter gergoviae, Proteus mirabilis, Proteus vulgaris, Providencia rettgeri, Providencia stuartii, Pseudomonas aeruginosa, Pseudomonas denitrificans (nomen rejiciendum), Pseudomonas fluorescens, Pseudomonas nitroreducens, Pseudomonas putida, Pseudomonas stutzeri, Raoultella ornithinolytica, Raoultella planticola, Salmonella enterica, Serratia ficaria, Serratia fonticola, Serratia grimesii, Serratia liquefaciens, Serratia marcescens, Serratia odorifera, Serratia plymuthica, Serratia proteamaculans, Serratia rubidaea, Shigella boydii, Shigella dysenteriae, Shigella flexneri, Shigella sonnei, Staphylococcus aureus, Staphylococcus cohnii, Staphylococcus epidermidis, Staphylococcus equorum, Staphylococcus haemolyticus, Staphylococcus saprophyticus, Stenotrophomonas maltophilia.

The classes of antimicrobial drug can be one selected from the group consisting of lactams, such as monobactams, β-lactams, carbapenems; β-lactam inhibitors; penicillins; quinolones and derivatives thereof, preferably fluoroquinolones; aminoglycosides; polyketides; benzene-derived compounds, sulfonamides; tetracyclines; cephalosporins; lincosamides; macrolides; nitrofuranes; glycopeptides; oxazolidinones; ansamycins; carbacephems; and folate synthesis inhibitors. The antimicrobial drugs can be one selected from the group consisting of amoxicillin/K clavulanate (AUG), ampicillin (AM), ampicillin/sulbactam (A/S), aztreonam (AZT), cefazolin (CFZ), cefepime (CPE), cefotaxime (CFT), ceftazidime (CAZ), ceftriaxone (CAX), cefuroxime (CRM), cephalotin (CF), ciprofloxacin (CP), ertapenem (ETP), gentamicin (GM), imipenem (IMP), levofloxacin (LVX), meropenem (MER), piperacillin/tazobactam (P/T), tetracycline (TE), tobramycin (TO), and trimethoprim/sulfamethoxazole (T/S).

In an embodiment, the microorganism is obtained from a patient, preferably from a patient suffering from an infection caused or suspected of being caused by the microorganism.

In an embodiment, the determination of the presence or absence of the nucleotide sequence and/or difference (AMR) is by high throughput sequencing.

In an embodiment, the present invention also involves a method for determining whether a patient is infected with a microorganism potentially resistant to an antimicrobial drug, which method comprises:
(i) determining the presence or absence in a microorganism, contained or suspected of being contained in a sample obtained from the patient, of at least one nucleotide sequence identified in Table I or a variant of the nucleotide sequence, and/or
(ii) determining the presence or absence in the microorganism of at least one difference in at least one nucleotide sequence identified in Table I compared with a reference sequence,
wherein the presence or absence of the at least one nucleotide sequence and/or difference indicates that the patient is infected with an antimicrobial drug resistant microorganism.

In an embodiment, the present invention involves a method of selecting a treatment for a patient suffering from an infection with an antimicrobial drug resistant microorganism, which method comprises:
(a)(i) determining the presence or absence in a microorganism obtained from the patient of at least one nucleotide sequence identified in Table I or a variant of the nucleotide sequence, and/or (a)(ii) determining the presence or absence in the microorganism of at least one difference in at least one nucleotide sequence identified in Table I compared with a reference sequence;
(b) identifying the one or more antimicrobial drugs to which the microorganism is resistant; and
(c) selecting one or more antimicrobial drugs different from those identified in step (b) and to which the microorganism is not resistant. Optionally, the method further comprises administering to the patient one or more of the antimicrobial drugs selected in step (c).

In certain embodiments, the methods can comprise:
(1) (i) determining the presence and/or absence in Acinetobacter spp. of at least one nucleotide sequence identified in rows 0 to 183, 282 to 289, and 291 to 325 of Table I or a variant of the nucleotide sequence, and/or (ii) determining the presence in Acinetobacter spp., of at least one difference in at least one nucleotide sequence identified in rows 184 to 281, 290, and 326 to 330 of Table I compared with a reference sequence,
(2) (i) determining the presence and/or absence in Escherichia spp. of at least one nucleotide sequence identified in rows 1248 to 1499 of Table I or a variant of the nucleotide sequence, and/or (ii) determining the presence in Escherichia spp. of at least one difference in at least one nucleotide sequence identified in rows 1500 to 1586 of Table I compared with a reference sequence,
(3) (i) determining the presence and/or absence in Klebsiella spp. of at least one nucleotide sequence identified in rows 1587 to 1696, 1743 to 1782, 1792 to 2133, 2208 to 2396, 2425 to 2427, and 2431 to 2446 of Table I or a variant of the nucleotide sequence, and/or (ii) determining the presence and/or absence in Klebsiella spp., of at least one difference in at least one nucleotide sequence identified in rows 1697 to 1742, 1783 to 1791, 2134 to 2207, 2397 to 2424, 2428 to 2430, 2447, and 2448 of Table I compared with a reference sequence,
(4) (i) determining the presence and/or absence in Morganella spp. of at least one nucleotide sequence identified in rows 2460 to 2569 of Table I or a variant of the nucleotide sequence, and/or (ii) determining the presence and/or absence in Morganella spp., of at least one difference in at least one nucleotide sequence identified in rows 2570 to 2621 of Table I compared with a reference sequence,
(5) (i) determining the presence and/or absence in Proteus spp. of at least one nucleotide sequence identified in rows 2623 to 2754, and 2789 to 2792 of Table I or a variant of the nucleotide sequence, and/or (ii) determining the presence and/or absence in Proteus spp., of at least one difference in at least one nucleotide sequence identified in rows 2755 to 2788 of Table I compared with a reference sequence,
(6) (i) determining the presence and/or absence in Pseudomonas spp. of at least one nucleotide sequence identified in rows 2793 to 2861, 2877, and 2878 of Table I or a variant of the nucleotide sequence, and/or (ii) determining the presence and/or absence in Pseudomonas spp., of at least one difference in at least one nucleotide sequence identified in rows 2862 to 2876 of Table I compared with a reference sequence,
(7) (i) determining the presence and/or absence in Salmonella spp. of at least one nucleotide sequence identified in rows 2885 to 2990 of Table I or a variant of the nucleotide sequence, and/or (ii) determining the presence in Salmonella spp., of at least one difference in at least one nucleotide sequence identified in rows 2991 to 3008 of Table I compared with a reference sequence,
(8) (i) determining the presence and/or absence in Serratia spp. of at least one nucleotide sequence identified in rows 3009 to 3013, 3023 to 3028, 3031 to 3131, and 3188 to 3198 of Table I or a variant of the nucleotide sequence, and/or (ii) determining the presence and/or absence in Serratia spp., of at least one difference in at least one nucleotide sequence identified in rows 3014 to 3022, 3029, 3030, 3132 to 3187, 3199, and 3200 of Table I compared with a reference sequence,
(9) (i) determining the presence in Stenotrophomonas spp. of at least one nucleotide sequence identified in rows 3746 to 3751 of Table I or a variant of the nucleotide sequence,
(10) (i) determining the presence and/or absence in Shigella spp. of at least one nucleotide sequence identified in rows 3201 to 3234, 3237 to 3256, and 3263 to 3352 of Table I or a variant of the nucleotide sequence, and/or (ii) determining the presence in Shigella spp., of at least one difference in at least one nucleotide sequence identified in rows 3235, 3236, 3257 to 3262, and 3353 to 3375 of Table I compared with a reference sequence,
(11) (i) determining the presence and/or absence in Enterobacter spp. of at least one nucleotide sequence identified in rows 692 to 697, 702 to 1074, 1190 to 1201, 1213 to 1226, 1231 to 1233, 1235 to 1239, 1246, and 1247 of Table I or a variant of the nucleotide sequence, and/or (ii) determining the presence and/or absence in Enterobacter spp., of at least one difference in at least one nucleotide sequence identified in rows 698 to 701, 1075 to 1189, 1202 to 1212, 1227 to 1230, 1234, and 1240 to 1245 of Table I compared with a reference sequence,
(12) (i) determining the presence and/or absence in Staphylococcus spp. of at least one nucleotide sequence identified in rows 3376 to 3664 of Table I or a variant of the nucleotide sequence, and/or (ii) determining the presence in Staphylococcus spp., of at least one difference in at least one nucleotide sequence identified in rows 3665 to 3745 of Table I compared with a reference sequence,
(13) (i) determining the presence and/or absence in Burkholderia spp. of at least the nucleotide sequence identified in row 331 of Table I or a variant of the nucleotide sequence,
(14) (i) determining the presence and/or absence in Citrobacter spp. of at least one nucleotide sequence identified in rows 332 to 342, 345 to 361, 365 to 419, 422 to 581, 634 to 650, 653 to 683, 690, and 691 of Table I or a variant of the nucleotide sequence, and/or (ii) determining the presence in Citrobacter spp., of at least one difference in at least one nucleotide sequence identified in rows 343, 344, 362 to 364, 420, 421, 582 to 633, 651, 652, and 684 to 689 of Table I compared with a reference sequence,
(15) (i) determining the presence and/or absence in Kluyvera spp. of at least one nucleotide sequence identified in rows 2449 to 2457 of Table I or a variant of the nucleotide sequence, and/or (ii) determining the presence in Kluyvera spp., of at least one difference in at least one nucleotide sequence identified in rows 2458 to 2459 of Table I compared with a reference sequence,
(16) (i) determining the presence and/or absence in Pantoea spp. of at least the nucleotide sequence identified in row 2622 of Table I or a variant of the nucleotide sequence, and/or
(17) (i) determining the presence and/or absence in Raoultella spp. of at least one nucleotide sequence identified in rows 2879 to 2881, and 2883 of Table I or a variant of the nucleotide sequence, and/or (ii) determining the presence in Raoultella spp., of at least one difference in at least one nucleotide sequence identified in rows 2882, and 2884 of Table I compared with a reference sequence.

In certain embodiments of the methods disclosed herein, in addition to the use of the AMRs identified in rows 0 to 3840 whose presence or absence is determined to determine or predict antimicrobial resistance of a microorganism:

(1) when determining the presence and/or absence in Acinetobacter spp. of the at least one nucleotide sequence and/or the at least one difference in at least one nucleotide sequence, the at least one nucleotide sequence can further include those identified in rows 3765 to 3840 of Table I;

(2) when determining the presence and/or absence in Escherichia spp. of the at least one nucleotide sequence and/or the at least one difference in at least one nucleotide sequence, the at least one nucleotide sequence and/or the at least one difference can further include those identified in rows 3752 to 3777 and 3785 to 3840 of Table I;

(3) when determining the presence and/or absence in Klebsiella spp. of the at least one nucleotide sequence and/or the at least one difference in at least one nucleotide sequence, the at least one nucleotide sequence and/or the at least one difference can further include those identified in rows 3752 to 3784 and 3798 to 3840 of Table I;

(4) when determining the presence and/or absence in Morganella spp. of the at least one nucleotide sequence and/or the at least one difference in at least one nucleotide sequence, the at least one nucleotide sequence and/or the at least one difference can further include those identified in rows 3752 to 3797 and 3799 to 3840 of Table I;

(5) when determining the presence and/or absence in Proteus spp. of the at least one nucleotide sequence and/or the at least one difference in at least one nucleotide sequence, the at least one nucleotide sequence and/or the at least one difference can further include those identified in rows 3752 to 3798 and 3808 to 3840 of Table I;

(6) when determining the presence and/or absence in Pseudomonas spp. of the at least one nucleotide sequence and/or the at least one difference in at least one nucleotide sequence, the at least one nucleotide sequence and/or the at least one difference can further include those identified in rows 3752 to 3807 and 3810 to 3840 of Table I;

(7) when determining the presence and/or absence in Salmonella spp. of the at least one nucleotide sequence and/or the at least one difference in at least one nucleotide sequence, the at least one nucleotide sequence and/or the at least one difference can further include those identified in rows 3752 to 3809 and 3816 to 3840 of Table I;

(8) when determining the presence and/or absence in Serratia spp. of the at least one nucleotide sequence and/or the at least one difference in at least one nucleotide sequence, the at least one nucleotide sequence and/or the at least one difference can further include those identified in rows 3752 to 3815 and 3817 to 3840 of Table I;

(9) when determining the presence and/or absence in Stenotrophomonas spp. of the at least one nucleotide sequence and/or the at least one difference in at least one nucleotide sequence, the at least one nucleotide sequence and/or the at least one difference can further include those identified in rows 3752 to 3839 of Table I;

(10) when determining the presence and/or absence in Shigella spp. of the at least one nucleotide sequence and/or the at least one difference in at least one nucleotide sequence, the at least one nucleotide sequence and/or the at least one difference can further include those identified in rows 3752 to 3816 and 3824 to 3840 of Table I;

(11) when determining the presence and/or absence in Enterobacter spp. of the at least one nucleotide sequence and/or the at least one difference in at least one nucleotide sequence, the at least one nucleotide sequence and/or the at least one difference can further include those identified in rows 3752 to 3767 and 3778 to 3840 of Table I;

(12) when determining the presence and/or absence in Staphylococcus spp. of the at least one nucleotide sequence and/or the at least one difference in at least one nucleotide sequence, the at least one nucleotide sequence and/or the at least one difference can further include those identified in rows 3752 to 3823 and 3840 of Table I; and/or

(13) when determining the presence and/or absence in Citrobacter spp. of the at least one nucleotide sequence and/or the at least one difference in at least one nucleotide sequence, the at least one nucleotide sequence and/or the at least one difference can further include those identified in rows 3752 to 3764 and 3768 to 3840 of Table I.

In certain embodiments, the methods disclosed herein entail the use of at least 1, 2, 3, 4, 5, 6, 7, 8, 9, 10, 15, 20, 30, 40, 50, 60, 70, 80, 90, 100, 150, 200, 250, 300, 400, 500, 600, 700, 800, 900, 1000, 1500, 2000, 2500, 3000, 3250 of the AMRs identified in Table 1. In other embodiments, the methods disclosed herein entail the use of at least 1, 2, 3, 4, 5, 6, 7, 8, 9, 10, 15, 20, 30, 40, 50, 60, 70, 80, 90, 100, 150, 200, 250, 300, 400, 500, 600, 700, 800, 900, 1000, 1500, 2000, 2500, 3000, 3250 of the AMRs identified in Table I, except:

(1) when determining the presence and/or absence in Acinetobacter spp., the at least one nucleotide sequence and/or the at least one difference excludes those identified in rows 3752 to 3763 of Table I;

(2) when determining the presence and/or absence in Escherichia spp., the at least one nucleotide sequence and/or the at least one difference excludes those identified in rows 3778 to 3784 of Table I;

(3) when determining the presence and/or absence in Klebsiella spp., the at least one nucleotide sequence and/or the at least one difference excludes those identified in rows 3785 to 3797 of Table I;

(4) when determining the presence and/or absence in Morganella spp., the at least one nucleotide sequence and/or the at least one difference excludes that identified in row 3798 of Table I;

(5) when determining the presence and/or absence in Proteus spp., the at least one nucleotide sequence and/or the at least one difference excludes those identified in rows 3799 to 3807 of Table I;

(6) when determining the presence and/or absence in Pseudomonas spp., the at least one nucleotide sequence and/or the at least one difference excludes those identified in rows 3808 and 3809 of Table I;

(7) when determining the presence and/or absence in Salmonella spp., the at least one nucleotide sequence and/or the at least one difference excludes those identified in rows 3810 to 3815 of Table I;

(8) when determining the presence and/or absence in Serratia spp., the at least one nucleotide sequence and/or the at least one difference excludes that identified in row 3816 of Table I;

(9) when determining the presence and/or absence in Stenotrophomonas spp., the at least one nucleotide sequence and/or the at least one difference excludes that identified in row 3840 of Table I;

(10) when determining the presence and/or absence in Shigella spp., the at least one nucleotide sequence and/or the at least one difference excludes those identified in rows 3817 to 3823 of Table I;

(11) when determining the presence and/or absence in Enterobacter spp., the at least one nucleotide sequence and/or the at least one difference excludes those identified in rows 3768 to 3777 of Table I;

(12) when determining the presence and/or absence in Staphylococcus spp., the at least one nucleotide sequence and/or the at least one difference excludes those identified in rows 3824 to 3839 of Table I; and/or

(13) when determining the presence and/or absence in Citrobacter spp., the at least one nucleotide sequence and/or the at least one difference excludes those identified in rows 3765 to 3767 of Table I.

In an embodiment, the methods of the invention preferably entail the use of 1, 2, 3, 4, 5, 6, 7, 8, 9, 10 or more of those AMRs identified with a + in the column of Table I entitled Marker associated with Cross-Resistance. In an embodiment, the methods of the invention preferably entail the use of 1, 2, 3, 4, 5, 6, 7, 8, 9, 10 or more of those AMRs identified with a + in the column of Table I entitled Associated with resistance in multiple pathogens. In an embodiment, the methods of the invention preferably entail the use of 1, 2, 3, 4, 5, 6, 7, 8, 9, 10 or more of those AMRs identified with a + in the column of Table I entitled cross-resistance in multiple pathogens. In an embodiment, a combination of at least 2 AMRs identified with a + from at least two of the aforementioned columns in Table I (at least one from each column) can be used in the methods described herein.

### DETAILED DESCRIPTION OF THE INVENTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Preferably, the terms used herein are defined as described in “A multilingual glossary of biotechnological terms: (IUPAC Recommendations)”, H. G. W. Leuenberger, B. Nagel, and H. Kölbl, Eds., (1995) Helvetica Chimica Acta, CH-4010 Basel, Switzerland.

The practice of the present invention will employ, unless otherwise indicated, conventional methods of biochemistry, cell biology, immunology, and recombinant DNA techniques which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps although in some embodiments such other member, integer or step or group of members, integers or steps may be excluded, i.e., the subject-matter consists in the inclusion of a stated member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed No language in the specification should be construed as indicating any non-claimed element essential to the practice of the invention.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

In an embodiment of the invention, a method for determining or predicting the antimicrobial drug resistance status of a microorganism is provided, which method comprises (i) determining the presence or absence in the microorganism of at least one nucleotide sequence identified in Table I or a variant of the nucleotide sequence, and/or (ii) determining the presence or absence in the microorganism of at least one difference in at least one nucleotide sequence identified in Table I compared with a reference sequence, wherein the presence or absence of the at least one nucleotide sequence and/or difference indicates the antimicrobial drug resistance status of the microorganism, i.e., that the microorganism is or is not resistant to one or more antimicrobial drugs or types/classes of antimicrobial drugs. In a preferred embodiment, the presence or absence of the at least one nucleotide sequence and/or difference indicates the microorganism is resistant to one or more antimicrobial drugs or to one or more types/classes of antimicrobial drugs.

In an embodiment, a diagnostic method for determining whether a patient is infected with a microorganism potentially resistant to an antimicrobial drug is provided, which method comprises (i) determining the presence or absence in a microorganism, contained or suspected of being contained in a sample obtained from the patient, of at least one nucleotide sequence identified in Table I or a variant of the nucleotide sequence, and/or (ii) determining the presence or absence in the microorganism of at least one difference in at least one nucleotide sequence identified in Table I compared with a reference sequence, wherein the presence or absence of the at least one nucleotide sequence and/or difference indicates that the patient is infected with an antimicrobial drug resistant microorganism.

In an embodiment, a method of selecting a treatment for a patient suffering from an infection with an antimicrobial drug resistant microorganism is provided, which method comprises (a)(i) determining the presence or absence in a microorganism, contained or suspected of being contained in a sample obtained from the patient, of at least one nucleotide sequence identified in Table I or a variant of the nucleotide sequence, and/or (a)(ii) determining the presence or absence in the microorganism of at least one difference in at least one nucleotide sequence identified in Table I compared with a reference sequence; (b) identifying the one or more antimicrobial drugs to which the microorganism is resistant; and (c) selecting one or more antimicrobial drugs different from those identified in step (b) and to which the microorganism is not resistant. The method can further comprise a step of administering the selected one or more antimicrobial drugs to the patient, e.g., in a method of treatment. The patient can be a vertebrate, preferably a mammal, for example, human, dog, cat, pig, horse, cattle, sheep, goat, mouse, or rat, preferably the patient is human.

In an embodiment, the at least one nucleotide sequence and/or the at least one difference in nucleotide sequence, the presence or absence of which in step (i) and/or (ii) is determined, is identified in rows 0 to 3840 of Table I, preferably in rows 0 to 3751 of Table I.

In an embodiment, the at least one nucleotide sequence and/or the at least one difference in nucleotide sequence, the presence or absence of which in step (i) and/or (ii) is determined, identified in the following rows of Table I is excluded in the methods of the invention: 3752 to 3840.

Exemplary antimicrobial drug classes include, but are not limited to lactams, such as monobactams, β-lactams, carbapenems; β-lactam inhibitors; penicillins; quinolones and derivatives thereof, preferably fluoroquinolones; aminoglycosides; polyketides; benzene-derived compounds, sulfonamides; tetracyclines; cephalosporins; lincosamides; macrolides; nitrofuranes; glycopeptides; oxazolidinones; ansamycins; carbacephems; and folate synthesis inhibitors. Exemplary antimicrobial drugs falling within the above classes include, but are not limited to amoxicillin/K clavulanate (AUG), ampicillin (AM), ampicillin/sulbactam (A/S), aztreonam (AZT), cefazolin (CFZ), cefepime (CPE), cefotaxime (CFT), ceftazidime (CAZ), ceftriaxone (CAX), cefuroxime (CRM), cephalotin (CF), ciprofloxacin (CP), ertapenem (ETP), gentamicin (GM), imipenem (IMP), levofloxacin (LVX), meropenem (MER), piperacillin/tazobactam (P/T), tetracycline (TE), tobramycin (TO), and trimethoprim/sulfamethoxazole (T/S).

In other embodiments, the method comprises determining the presence/absence of at least two, at least three, at least four, at least five, at least six, at least seven, at least eight, at least nine, at least ten, such as two or more, three or more, four or more, five or more, etc., nucleotide sequences/differences (AMRs). In an embodiment, the microorganism is the one identified in the same respective row as the nucleotide sequence/difference (AMR) in Table I and the antimicrobial drug to which resistance is associated is also identified in the same respective row as the nucleotide sequence/difference (AMR) in Table II. For example, for marker id AR000059 in row 17 of Table I, the marker when present, e.g., in Acinetobacter baumannii, indicates resistance to gentamicin in the aminoglycoside class of antimicrobial drugs, which indication for resistance has a p value of 4.2093×10⁻¹² as set forth in row 18 of Table II. This same maker when present, e.g., in Acinetobacter baumannii, also indicates resistance to ciprofloxacin in the fluoroquinolone class of antimicrobial drugs, which indication has a p value of 0.0245643 as set forth in row 1398 of Table II (see also row 1399 of Table II disclosing that the same marker indicates resistance to levofloxacin with a p value of 1.33364×10⁻¹²). This same marker when present also indicates resistance in Proteus mirabilis to, e.g., gentamicin, tobramycin, cefuroxime, cefotaxime, among others, see rows 37945, 37946, 38355, and 38505 of Table II. Thus, this marker not only indicates resistance to multiple antimicrobial drugs, but also to antimicrobial drugs in different classes and in different, unrelated microorganisms.

In another example, for marker id AR000503-G146* in row 1564 of Table I, when preferably a thymidine residue (T) is present at position 436 in the nucleotide sequence (a difference from the adenosine residue (A) present in the reference sequence), in, e.g., Escherichia coli, indicates resistance to ampicillin in the penicillin class of antimicrobial drugs, which indication has a p value of 4.07194×10⁻⁶ as set forth in row 22696 of Table II. Where the alternative amino acid is indicated by an asterix, this means that the change in the encoding nucleotide resulted in a stop codon and that the protein sequence terminates. In yet another example, for marker id AR003113-E729D in row 2764 of Table I, when preferably a thymidine residue (T) is present at position 2187 in the nucleotide sequence (a difference from the guanosine residue (G) present in the reference sequence), in, e.g., Proteus mirabilis, indicates resistance to tobramycin and ceftazidime, see rows 40092 and 40225 in Table II, respectively. This same marker also indicates resistance to multiple classes of antimicrobial drugs in Serratia marcescens, see rows 47084 (gentamicin) and 47271 (trimethoprim-sulfamethoxaole) in Table II.

In particular embodiments, the methods disclosed herein comprise determining the presence or absence of the at least one nucleotide sequence and determining the presence or absence of the at least one difference in a nucleotide sequence. In an embodiment, the methods of the invention can comprise determining the presence/absence of a combination of any number of nucleotide sequences/differences (AMRs). In an embodiment, only the nucleotide sequences/differences the presence of which is indicative of antimicrobial drug resistance are determined. In an embodiment, only the nucleotide sequences/differences the absence of which is indicative of antimicrobial drug resistance are determined.

In certain embodiments of the methods disclosed herein, antimicrobial drug resistance is indicated where the presence or absence of the difference in a particular nucleotide sequence can be any difference located at the position identified in Table I for that particular nucleotide sequence (nc_pos), i.e., any nucleotide except for the wild-type nucleotide. In certain embodiments of the methods disclosed herein, antimicrobial drug resistance is indicated where the presence or absence of the difference in a particular nucleotide sequence not only is located at the position identified in Table I for that particular nucleotide sequence but also where the specific difference at that position is as identified in Table I (nc_alt) for that particular nucleotide sequence. In an embodiment, the specific difference at the specific position also includes any difference in the same codon in which the specific position is located and which leads to the same or to a different change in the encoded amino acid sequence.

The present invention is based, in part, on the identification of antimicrobial resistance markers (AMRs), which when present or absent in a microorganism, are indicative of the microorganism being resistant to one or more antimicrobial drugs. In certain cases, the presence or absence of the AMR is directly responsible for the phenotypic change in resistance, in other cases, the presence or absence is statistically associated with such resistance. These markers are identified in Table I and have been identified in a number of different exemplary microorganisms, such as Achromobacter, Acinetobacter, Burkholderia, Citrobacter, Enterobacter, Escherichia, Klebsiella, Kluyvera, Lelliottia, Morganella, Pantoea, Pluralibacter, Proteus, Providencia, Pseudomonas, Raoultella, Salmonella, Serratia, Shigella, Staphylococcus, and Stenotrophomonas spp.

As used herein, a variant of a nucleotide sequence whose presence or absence is to be determined can be one that is at least 80%, at least 85%, at least 90%, at least 95%, at least 96%, at least 97%, at least 98%, or at least 99% identical over the entire length of the sequence compared to the corresponding nucleotide sequence disclosed herein. The variant can be a naturally occurring variant observed in other isolates of the sequence from the same microorganism, e.g., from the same genus or from the same species. In cases where the specified nucleotide sequence encodes a peptide/protein, the variant nucleotide sequence also will typically encode the same peptide/protein having essentially the same wild type amino acid sequence and activity. Essentially the same means that although there may be amino acid substitutions in the sequence, the encoded peptide/protein will have the same activity, though such activity may be increased or decreased. For example, the activity is decreased by no more than 5%, no more than 10%, no more than 15%, no more than 20%, no more than 25%, no more than 30%, no more than 35%, no more than 40%, no more than 45% or no more than 50%. Further, nucleotide sequences in which a difference in the sequence is to be determined can encompass variants of the sequence. A variant of the sequence can be one that is a naturally occurring variant in which there are differences at positions observed in other isolates of the sequence from the same microorganism, e.g., from the same genus or from the same species, other than the difference specified in Table I.

Table I identifies each AMR by an individual identifier number or a unique marker ID, and whether or not its presence or absence in the microorganism is indicative of (associated with) resistance of the microorganism to at least the listed antimicrobial drugs in Table II for that marker. For certain AMRs where the presence or absence of a difference in the nucleotide sequence is indicative of resistance, a preferred position (i.e., nc_pos or aa_pos) where the difference is located, as well as a preferred difference (i.e., nc_alt or as alt) in the sequence are disclosed in Table I. If there is any discrepancy between the identification number of an AMR in the text and in Tables I or II, the identification set forth in the tables is controlling. The abbreviated headings in Table I are “mrk_id”—marker ID; “nc_pos”/“aa_pos”—position in the nucleotide/amino acid sequence, respectively, of the difference; “nc_ref”/“aa_ref”—the wild-type nucleotide/amino acid residue, respectively, at that position; “nc_alt”/“aa_alt”—the preferred changed in the nucleotide/amino acid sequence, respectively, at that position whose presence or absence is associated with antimicrobial drug resistance.

Further, Table I in the column “Marker associated with Cross-Resistance” identifies the particular AMRs that are indicative of resistance to more than one class/type of antimicrobial drug, e.g., resistance to imipenem (a carbapenem) and tobramycin (an aminoglycoside), with the plus (+) symbol. “The minus (−) symbol means that the marker is indicative of resistance only to one class of antimicrobial drugs. In the column “Associated with resistance in multiple pathogens”, Table I identifies those markers with the plus (+) symbol that are associated with resistance in more than one type of microorganism. The minus (−) symbol means that the maker is indicative of resistance only in the one type of microorganism with which it is associated in Table I. In the column “cross-resistance in multiple pathogens”, Table I identifies those markers with the plus (+) symbol that are indicative of resistance to more than one class/type of antimicrobial drug in more than one type of microorganism. Any marker identified with a plus (+) symbol in one or more of these three columns is a preferred marker for use in the methods disclosed herein.

Table I also sets forth for each of the identified AMRs the sequence identifier number (SEQ ID NO) in the Sequence Listing for the nucleotide sequence (DNA Sequence) and its encoded amino acid sequence (PRT Sequence). The nucleotide and encoded amino acid sequences set forth in the Sequence Listing are the respective wild type/reference nucleotide and amino acid sequences, except where a difference in the sequence indicates antimicrobial drug resistance. In those AMRs, the preferred position of the difference is indicated by “n” in the nucleotide sequence and “Xaa” in the amino acid sequence.

In Table II, 25 drugs grouped into drug classes based on their category in the 2017 EUCAST guidelines (EUCAST. European Committee on Antimicrobial Susceptibility Testing Antifungal Agents Breakpoint tables for interpretation of MICs. Eucast 1-5) are disclosed: 5 drugs belong to cephalosporins (cefuroxime—2nd generation, cefotaxime, ceftazidime and ceftriaxone—3rd generation, cefepime—4th generation), 7 to penicillins, 3 to carbapenems, 3 to fluoroquinolones, 2 to aminoglycosides, in addition to 1 tetracycline, 1 monobactam, 1 folate pathway inhibitor, 1 lincosamide and 1 macrolide. It is important to note that even though Tables I and II deal with the same AMRs, the number of entries from Tables I will not match the number of entries in Table II since a single marker (single entry in Table I) can be reported as indicative of resistance against multiple compounds and in multiple microorganisms and, therefore, have more than one entry in Table II.

Table II also sets forth the performance of the respective marker in predicting resistance against the respective antimicrobial drug in a particular species using performance metrics such as sensitivity, specificity, accuracy, positive predictive value (ppv), and negative predictive value (npv).

As used herein, the reference sequence is the corresponding wild-type sequence in the microorganism for the particular nucleotide/amino acid sequence. The wild-type sequence can be the sequence found in same genus and/or same genus/species of the microorganism which is or is not resistant to the respective antimicrobial drug, depending on whether the presence or absence of the difference indicates resistance. The reference sequence can also be that disclosed in a reference database or be the result of sequencing a number of different isolates of the same microbial species where the sequence having the highest frequency is the reference sequence.

Where the microorganism is Acinetobacter spp., preferably Acinetobacter baumannii, Acinetobacter calcoaceticus, Acinetobacter haemolyticus, Acinetobacter nosocomialis, Acinetobacter pitti, or Acinetobacter sp. ADP1, the AMRs that when present or absent are indicative of resistance to at least one antimicrobial drug are preferably selected from those identified in rows 0 to 330 of Table I.

The AMRs can optionally further comprise those identified in rows 3752 to 3764 of Table I. A preferred difference in the nucleotide sequence identified in rows 184 to 281, 290, 326 to 330, and optionally in row 3764 of Table 1 is set forth in each of the same respective rows of Table I. The preferred at least one antimicrobial drug to which resistance is indicated by the presence or absence of the AMR is disclosed for the same respective marker ID of the AMR in Table II.

Those AMRs when present in Acinetobacter spp. which are indicative of resistance to at least one antimicrobial drug are preferably selected from those identified in rows 0 to 66, 73 to 83, 85 to 92, 94 to 99, 101, 103 to 108, 117 to 123, 125 to 127, 129 to 140, 142 to 145, 150 to 157, 161, 164, 166, 167, 170 to 183, 185, 186, 188, 190, 191, 195 to 202, 204, 205, 207 to 211, 213, 216 to 230, 234 to 247, 249 to 253, 255 to 263, 265, 267 to 270, and 272 to 282, 284, 285 to 290, 292 to 330 of Table I.

Those AMRs when absent in Acinetobacter spp. which are indicative of resistance to at least one antimicrobial drug are preferably selected from those identified in rows 67 to 72, 84, 93, 100, 102, 109 to 116, 124, 128, 141, 146 to 149, 158 to 160, 162, 163, 165, 168, 169, 184, 187, 189, 192 to 194, 203, 206, 212, 214, 215, 231 to 233, 248, 254, 264, 266, 271, 283, and 291 of Table I.

Those AMRs which involve a nucleotide sequence that when present in Acinetobacter spp. are indicative of resistance to at least one antimicrobial drug are preferably selected from those identified in rows 0 to 66, 73 to 83, 85 to 92, 94 to 99, 101, 103 to 108, 117 to 123, 125 to 127, 129 to 140, 142 to 145, 150 to 157, 161, 164, 166, 167, 170 to 183, 282, 284 to 289, 292 to 325, and 330 of Table I. Further, the AMRs which involve at least one difference in at least one nucleotide sequence compared to a reference sequence that when present are indicative of resistance to at least one antimicrobial drug are preferably selected from those identified in rows 185, 186, 188, 190, 191, 195 to 202, 204, 205, 207 to 211, 213, 216 to 230, 234 to 247, 249 to 253, 255 to 263, 265, 267 to 270, and 272 to 281, 290, and 326 to 329 in Table I.

Those AMRs which involve a nucleotide sequence that when absent in Acinetobacter spp. are indicative of resistance to at least one antimicrobial drug are preferably selected from those identified in rows 67 to 72, 84, 93, 100, 102, 109 to 116, 124, 128, 141, 146 to 149, 158 to 160, 162, 163, 165, 168, 169, 283, and 291 of Table I. Further, the AMRs which involve at least one difference in at least one nucleotide sequence compared to a reference sequence that when absent are indicative of resistance to at least one antimicrobial drug are preferably selected from those identified in rows 184, 187, 189, 192 to 194, 203, 206, 212, 214, 215, 231 to 233, 248, 254, 264, 266, and 271 in Table I.

Those AMRs when present in Acinetobacter baumannii which are indicative of resistance to at least one antimicrobial drug are preferably selected from those identified in rows 0 to 66, 73 to 83, 85 to 92, 94 to 99, 101, 103 to 108, 117 to 123, 125 to 127, 129 to 140, 142 to 145, 150 to 157, 161, 164, 166, 167, 170 to 183, 185, 186, 188, 190, 191, 195 to 202, 204, 205, 207 to 211, 213, 216 to 230, 234 to 247, 249 to 253, 255 to 263, 265, 267 to 270, and 272 to 281 of Table I. Those AMRs when absent in Acinetobacter baumannii which are indicative of resistance to at least one antimicrobial drug are preferably selected from those identified in rows 67 to 72, 84, 93, 100, 102, 109 to 116, 124, 128, 141, 146 to 149, 158 to 160, 162, 163, 165, 168, 169, 184, 187, 189, 192 to 194, 203, 206, 212, 214, 215, 231 to 233, 248, 254, 264, 266, and 271 of Table I.

Those AMRs when present in Acinetobacter calcoaceticus which are indicative of resistance to at least one antimicrobial drug are preferably selected from those identified in row 282 of Table I. Those AMRs when absent in Acinetobacter calcoaceticus which are indicative of resistance to at least one antimicrobial drug are preferably selected from those identified in row 283 of Table I.

Those AMRs when present in Acinetobacter haemolyticus which are indicative of resistance to at least one antimicrobial drug are preferably selected from those identified in row 284 of Table I.

Those AMRs when present in Acinetobacter nosocomialis which are indicative of resistance to at least one antimicrobial drug are preferably selected from those identified in rows 285 to 290 of Table I.

Those AMRs when present in Acinetobacter pitti which are indicative of resistance to at least one antimicrobial drug are preferably selected from those identified in rows 292 to 325 of Table I. Those AMRs when absent in Acinetobacter pitti which are indicative of resistance to at least one antimicrobial drug are preferably selected from those identified in row 291 of Table I.

Those AMRs when present in Acinetobacter sp. ADP1 which are indicative of resistance to at least one antimicrobial drug are preferably selected from those identified in row 330 of Table I.

In embodiments where the Acinetobacter microorganism is a particular species of Acinetobacter, the methods preferably comprise determining the presence and/or absence of the AMRs determined to be indicative of antimicrobial drug resistance in that species set forth in Table I.

It is intended that the foregoing disclosure regarding the AMRs for Acinetobacter spp. be equally applicable to all of the methods disclosed herein, in particular those methods which comprise steps of determining the presence or absence of nucleotide sequences and/or differences in nucleotide sequences. In an embodiment, those AMRs for Acinetobacter spp. having p values not greater than 0.1, 0.01, 0.001, 0.0001, 10⁻⁵, 10⁻¹⁰, or 10⁻²⁰ as set forth in Table II are the AMRs used in the methods disclosed herein to indicate antimicrobial drug resistance. In an embodiment, those AMRs for Acinetobacter spp. having ppv values greater than 0.7, 0.75, 0.8, 0.85, 0.9, 0.95, or 0.98 as set forth in Table II are the AMRs used in the methods disclosed herein to indicate antimicrobial drug resistance. In an embodiment, those AMRs for Acinetobacter spp. having p values not greater than 0.1, 0.01, 0.001, 0.0001, 10⁻⁵, 10⁻¹⁰, or 10⁻²⁰ as set forth in Table II and having ppv values greater than 0.7, 0.75, 0.8, 0.85, 0.9, 0.95, or 0.98 as set forth in Table II are the AMRs used in the methods disclosed herein to indicate antimicrobial drug resistance.

In accordance with the foregoing, in an exemplary embodiment, where the microorganism is Acinetobacter, the methods disclosed herein comprise (i) determining the presence and/or absence in Acinetobacter spp. of at least one nucleotide sequence identified in rows 0 to 183, 282 to 289, 291 to 325, and 330 of Table I or a variant of the nucleotide sequence, and/or (ii) determining the presence in Acinetobacter spp., of at least one difference in at least one nucleotide sequence identified in rows 184 to 281, 290, and 326 to 330 of Table I compared with a reference sequence.

In an embodiment, the method comprises determining the presence in Acinetobacter spp. of at least one nucleotide sequence identified in rows 0 to 66, 73 to 83, 85 to 92, 94 to 99, 101, 103 to 108, 117 to 123, 125 to 127, 129 to 140, 142 to 145, 150 to 157, 161, 164, 166, 167, 170 to 183, 282, 284 to 289, 292 to 325, and 330 of Table I or a variant of the nucleotide sequence. In an embodiment, the method comprises determining the presence in Acinetobacter spp. of at least one difference in at least one nucleotide sequence identified in rows 185, 186, 188, 190, 191, 195 to 202, 204, 205, 207 to 211, 213, 216 to 230, 234 to 247, 249 to 253, 255 to 263, 265, 267 to 270, and 272 to 281, 290, and 326 to 329 of Table I compared with a reference sequence.

In an embodiment, the method comprises determining the absence in Acinetobacter spp. of at least one nucleotide sequence identified in rows 67 to 72, 84, 93, 100, 102, 109 to 116, 124, 128, 141, 146 to 149, 158 to 160, 162, 163, 165, 168, 169, 283, and 291 of Table I or a variant of the nucleotide sequence. In an embodiment, the method comprises determining the absence in Acinetobacter spp. of at least one difference in at least one nucleotide sequence identified in rows 184, 187, 189, 192 to 194, 203, 206, 212, 214, 215, 231 to 233, 248, 254, 264, 266, and 271 of Table I compared with a reference sequence.

Where the microorganism is Citrobacter spp., preferably Citrobacter amalonaticus, Citrobacter braakii, Citrobacter freundii, Citrobacter koseri, Citrobacter pasteurii, Citrobacter portucalensis, Citrobacter rodentium, or Citrobacter werkmanii, the AMRs that when present or absent are indicative of resistance to at least one antimicrobial drug are preferably selected from those identified in rows 332 to 691 of Table I. The AMRs can optionally further comprise those identified in rows 3765 to 3767 of Table I. A preferred difference in the nucleotide sequence identified in rows 343, 344, 362 to 364, 420, 421, 582 to 633, 651, 652, and 684 to 689 of Table 1 is set forth in each of the same respective rows of Table I. The preferred at least one antimicrobial drug to which resistance is indicated by the presence or absence of the AMR is disclosed for the same respective marker ID of the AMR in Table II.

Those AMRs when present in Citrobacter spp. which are indicative of resistance to at least one antimicrobial drug are preferably selected from those identified in rows 332 to 344, 347 to 406, 408 to 419, 421 to 443, 445 to 449, 451 to 453, 455 to 476, 478 to 521, 525 to 608, 610 to 614, 616, 618 to 632, 634, 638, 639, 642 to 651, 653 to 655, 657, 659, 660, 663 to 667, 671 to 673, 675 to 680, and 688 to 691 of Table I.

Those AMRs when absent in Citrobacter spp. which are indicative of resistance to at least one antimicrobial drug are preferably selected from those identified in rows 345, 346, 407, 420, 444, 450, 454, 477, 522 to 524, 609, 615, 617, 633, 635 to 637, 640, 641, 652, 656, 658, 661, 662, 669, 670, 674, and 681 to 687 of Table I.

Those AMRs which involve a nucleotide sequence that when present in Citrobacter spp. are indicative of resistance to at least one antimicrobial drug are preferably selected from those identified in rows 332 to 342, 347 to 361, 365 to 406, 408 to 419, 422 to 443, 445 to 449, 451 to 453, 455 to 476, 478 to 521, 525 to 581, 634, 638, 639, 642 to 650, 653 to 655, 657, 659, 660, 663 to 667, 671 to 673, 675 to 680, 690, and 691 of Table I. Further, the AMRs which involve at least one difference in at least one nucleotide sequence compared to a reference sequence that when present are indicative of resistance to at least one antimicrobial drug are preferably selected from those identified in rows 343, 344, 362 to 364, 421, 582 to 608, 610 to 614, 616, 618 to 632, 651, 688 and 689 in Table I.

Those AMRs which involve a nucleotide sequence that when absent in Citrobacter spp. are indicative of resistance to at least one antimicrobial drug are preferably selected from those identified in rows 345, 346, 444, 450, 454, 477, 522 to 524, 635 to 637, 640, 641, 656, 658, 661, 662, 669, 670, 674, and 681 to 683 of Table I. Further, the AMRs which involve at least one difference in at least one nucleotide sequence compared to a reference sequence that when absent are indicative of resistance to at least one antimicrobial drug are preferably selected from those identified in rows 609, 615, 617, 633, 652, and 684 to 687 in Table I.

Those AMRs when present in Citrobacter amalonaticus which are indicative of resistance to at least one antimicrobial drug are preferably selected from those identified in rows 332 to 344 of Table I.

Those AMRs when present in Citrobacter braakii which are indicative of resistance to at least one antimicrobial drug are preferably selected from those identified in rows 347 to 364 of Table I. Those AMRs when absent in Citrobacter braakii which are indicative of resistance to at least one antimicrobial drug are preferably selected from those identified in rows 345 and 346 of Table I.

Those AMRs when present in Citrobacter freundii which are indicative of resistance to at least one antimicrobial drug are preferably selected from those identified in rows 365 to 406, 408 to 419, and 421 of Table I. Those AMRs when absent in Citrobacter freundii which are indicative of resistance to at least one antimicrobial drug are preferably selected from those identified in rows 407 and 420 of Table I.

Those AMRs when present in Citrobacter koseri which are indicative of resistance to at least one antimicrobial drug are preferably selected from those identified in rows 422 to 443, 445 to 449, 451 to 453, 455 to 476, 478 to 521, 525 to 608, 610 to 614, 616, 618 to 632 of Table I. Those AMRs when absent in Citrobacter koseri which are indicative of resistance to at least one antimicrobial drug are preferably selected from those identified in rows 444, 450, 454, 477, 522 to 524, 609, 615, 617, and 633 of Table I.

Those AMRs when present in Citrobacter pasteurii which are indicative of resistance to at least one antimicrobial drug are preferably selected from those identified in rows 634, 638, 639, and 642 to 651 of Table I. Those AMRs when absent in Citrobacter pasteurii which are indicative of resistance to at least one antimicrobial drug are preferably selected from those identified in rows 635 to 637, 640, 641, and 652 of Table I.

Those AMRs when present in Citrobacter portucalensis which are indicative of resistance to at least one antimicrobial drug are preferably selected from those identified in rows 653 to 655, and 657 of Table I. Those AMRs when absent in Citrobacter portucalensis which are indicative of resistance to at least one antimicrobial drug are preferably selected from those identified in rows 656 of Table I.

Those AMRs when present in Citrobacter rodentium which are indicative of resistance to at least one antimicrobial drug are preferably selected from those identified in rows 659, 660, 663 to 667, 671 to 673, 675 to 680, 688, and 689 of Table I. Those AMRs when absent in Citrobacter rodentium which are indicative of resistance to at least one antimicrobial drug are preferably selected from those identified in rows 658, 661, 662, 669, 670, 674, and 681 to 687 of Table I.

Those AMRs when present in Citrobacter werkmanii which are indicative of resistance to at least one antimicrobial drug are preferably selected from those identified in rows 690 and 691 of Table I.

In embodiments where the Citrobacter microorganism is a particular species of Citrobacter, the methods preferably comprise determining the presence and/or absence of the AMRs determined to be indicative of antimicrobial drug resistance in that species set forth in Table I.

It is intended that the foregoing disclosure regarding the AMRs for Citrobacter spp. be equally applicable to all of the methods disclosed herein, in particular those methods which comprise steps of determining the presence or absence of nucleotide sequences and/or differences in nucleotide sequences.

In an embodiment, those AMRs for Citrobacter spp. having p values not greater than 0.1, 0.01, 0.001, 0.0001, 10⁻⁵, 10⁻¹⁰, or 10⁻²⁰ as set forth in Table II are the AMRs used in the methods disclosed herein to indicate antimicrobial drug resistance. In an embodiment, those AMRs for Acinetobacter spp. having ppv values greater than 0.7, 0.75, 0.8, 0.85, 0.9, 0.95, or 0.98 as set forth in Table II are the AMRs used in the methods disclosed herein to indicate antimicrobial drug resistance. In an embodiment, those AMRs for Citrobacter spp. having p values not greater than 0.1, 0.01, 0.001, 0.0001, 10⁻⁵, 10⁻¹⁰, or 10⁻²⁰ as set forth in Table II and having ppv values greater than 0.7, 0.75, 0.8, 0.85, 0.9, 0.95, or 0.98 as set forth in Table II are the AMRs used in the methods disclosed herein to indicate antimicrobial drug resistance.

In accordance with the foregoing, in an exemplary embodiment, where the microorganism is Citrobacter, the methods disclosed herein comprise (i) determining the presence and/or absence in Citrobacter spp. of at least one nucleotide sequence identified in rows 332 to 342, 347 to 361, 365 to 406, 408 to 419, 422 to 443, 445 to 449, 451 to 453, 455 to 476, 478 to 521, 525 to 581, 634, 638, 639, 642 to 650, 653 to 655, 657, 659, 660, 663 to 667, 671 to 673, 675 to 680, 690, and 691 of Table I or a variant of the nucleotide sequence, and/or (ii) determining the presence and/or absence in Citrobacter spp., of at least one difference in at least one nucleotide sequence identified in rows 343, 344, 362 to 364, 421, 582 to 608, 610 to 614, 616, 618 to 632, 651, 688 and 689 of Table I compared with a reference sequence.

In an embodiment, the method comprises determining the presence in Citrobacter spp. of at least one nucleotide sequence identified in rows 332 to 342, 347 to 361, 365 to 406, 408 to 419, 422 to 443, 445 to 449, 451 to 453, 455 to 476, 478 to 521, 525 to 581, 634, 638, 639, 642 to 650, 653 to 655, 657, 659, 660, 663 to 667, 671 to 673, 675 to 680, 690, and 691 of Table I or a variant of the nucleotide sequence. In an embodiment, the method comprises determining the presence in Citrobacter spp. of at least one difference in at least one nucleotide sequence identified in rows 343, 344, 362 to 364, 421, 582 to 608, 610 to 614, 616, 618 to 632, 651, 688 and 689 of Table I compared with a reference sequence.

In an embodiment, the method comprises determining the absence in Citrobacter spp. of at least one nucleotide sequence identified in rows 345, 346, 444, 450, 454, 477, 522 to 524, 635 to 637, 640, 641, 656, 658, 661, 662, 669, 670, 674, and 681 to 683 of Table I or a variant of the nucleotide sequence. In an embodiment, the method comprises determining the absence in Citrobacter spp. of at least one difference in at least one nucleotide sequence identified in rows 609, 615, 617, 633, 652, and 684 to 687 of Table I compared with a reference sequence.

Where the microorganism is Escherichia spp., preferably Escherichia coli, the AMRs that when present or absent are indicative of resistance to at least one antimicrobial drug are preferably selected from those identified in rows 1248 to 1586 of Table I. The AMRs can optionally further comprise those identified in rows 3778 to 3784 of Table I. A preferred difference in the nucleotide sequence identified in rows 1500 to 1586 of Table I is set forth in each of the same respective rows of Table I. The preferred at least one antimicrobial drug to which resistance is indicated by the presence of the AMR is disclosed for the same respective marker ID of the AMR in Table II.

Those AMRs which involve a nucleotide sequence that when present in Escherichia spp. are indicative of resistance to at least one antimicrobial drug are preferably selected from those identified in rows 1248 to 1253, 1255 to 1484, 1487 to 1499 of Table I. Further, the AMRs which involve at least one difference in at least one nucleotide sequence compared to a reference sequence that when present are indicative of resistance to at least one antimicrobial drug are preferably selected from those identified in rows 1500 to 1579 and 1583 to 1586 in Table I.

Those AMRs which involve a nucleotide sequence that when absent in Escherichia spp. are indicative of resistance to at least one antimicrobial drug are preferably selected from those identified in rows 1254, 1485, and 1486 of Table I. Further, the AMRs which involve at least one difference in at least one nucleotide sequence compared to a reference sequence that when absent are indicative of resistance to at least one antimicrobial drug are preferably selected from those identified in rows 1580 to 1582 in Table I.

It is intended that the foregoing disclosure regarding the AMRs for Escherichia spp. be equally applicable to all of the methods disclosed herein, in particular those methods which comprise steps of determining the presence or absence of nucleotide sequences and/or differences in nucleotide sequences. In an embodiment, those AMRs for Escherichia spp. having p values not greater than 0.1, 0.01, 0.001, 0.0001, 10⁻⁵, 10⁻¹⁰, or 10⁻²⁰ as set forth in Table II are the AMRs used in the methods disclosed herein to indicate antimicrobial drug resistance. In an embodiment, those AMRs for Escherichia spp. having ppv values greater than 0.7, 0.75, 0.8, 0.85, 0.9, 0.95, or 0.98 as set forth in Table II are the AMRs used in the methods disclosed herein to indicate antimicrobial drug resistance. In an embodiment, those AMRs for Escherichia spp. having p values not greater than 0.1, 0.01, 0.001, 0.0001, 10⁻⁵, 10⁻¹⁰, or 10⁻²⁰ as set forth in Table II and having ppv values greater than 0.7, 0.75, 0.8, 0.85, 0.9, 0.95, or 0.98 as set forth in Table II are the AMRs used in the methods disclosed herein to indicate antimicrobial drug resistance.

In accordance with the foregoing, in an exemplary embodiment, where the microorganism is Escherichia, the methods disclosed herein comprise (i) determining the presence and/or absence in Escherichia spp. of at least one nucleotide sequence identified in rows 1248 to 1499 of Table I or a variant of the nucleotide sequence, and/or (ii) determining the presence and/or absence in Escherichia spp. of at least one difference in at least one nucleotide sequence identified in rows 1500 to 1586 of Table I compared with a reference sequence.

In an embodiment, the method comprises determining the presence in Escherichia spp. of at least one nucleotide sequence identified in rows 1248 to 1253, 1255 to 1484, 1487 to 1499 of Table I or a variant of the nucleotide sequence. In an embodiment, the method comprises determining the presence in Escherichia spp. of at least one difference in at least one nucleotide sequence identified in rows 1500 to 1579 and 1583 to 1586 of Table I compared with a reference sequence.

In an embodiment, the method comprises determining the absence in Escherichia spp. of at least one nucleotide sequence identified in rows 1254, 1485, and 1486 of Table I or a variant of the nucleotide sequence. In an embodiment, the method comprises determining the absence in Escherichia spp. of at least one difference in at least one nucleotide sequence identified in rows 1580 to 1582 of Table I compared with a reference sequence.

Where the microorganism is Klebsiella spp., preferably Klebsiella aerogenes, Klebsiella michiganensis, Klebsiella oxytoca, Klebsiella pneumoniae, Klebsiella quasipneumoniae or Klebsiella variicola the AMRs that when present or absent are indicative of resistance to at least one antimicrobial drug are preferably selected from those identified in rows 1587 to 2448 of Table I. The AMRs can optionally further comprise those identified in rows 3785 to 3797 of Table I. A preferred difference in the nucleotide sequence identified in rows 1697 to 1742, 1783 to 1791, 2134 to 2207, 2397 to 2424, 2428 to 2430, 2447, and 2448 of Table 1 is set forth in each of the same respective rows of Table I. The preferred at least one antimicrobial drug to which resistance is indicated by the presence or absence of the AMR is disclosed for the same respective marker ID of the AMR in Table II.

Those AMRs when present in Klebsiella spp. which are indicative of resistance to at least one antimicrobial drug are preferably selected from those identified in rows 1587 to 1717, 1719 to 1736, 1739 to 1744, 1746 to 1749, 1752 to 1754, 1756 to 1765, 1767 to 1773, 1775 to 1780, 1782, 1784, 1786, 1788, 1789, 1792 to 1794, 1796, 1797, 1800, 1803, 1804, 1806, 1807, 1820, 1821, 1829 to 1834, 1836 to 1841, 1843 to 1864, 1866 to 1878, 1880 to 1887, 1889 to 1910, 1912 to 2027, 2034 to 2040, 2045 to 2051, 2058, 2059, 2061 to 2066, 2077, 2079, 2082, 2084 to 2090, 2093, 2094, 2100, 2110 to 2116, 2119 to 2121, 2132 to 2134, 2136, 2139 to 2148, 2150 to 2154, 2156 to 2171, 2173, 2175, 2177 to 2185, 2187, 2188, 2193, 2194, 2196 to 2199, 2202, 2204, 2206 to 2211, 2213 to 2222, 2264 to 2273, 2275 to 2380, 2382 to 2388, 2390 to 2397, 2401, 2403, 2409 to 2415, 2417 to 2420, 2422 to 2425, 2427 to 2431, 2441, 2442, and 2444 to 2447 of Table I.

Those AMRs when absent in Klebsiella spp. which are indicative of resistance to at least one antimicrobial drug are preferably selected from those identified in rows 1718, 1737, 1738, 1740, 1745, 1750, 1751, 1755, 1766, 1774, 1781, 1783, 1785, 1787, 1790, 1791, 1795, 1798, 1799, 1801, 1802, 1805, 1808 to 1819, 1822 to 1828, 1835, 1842, 1865, 1879, 1888, 1911, 2028 to 2033, 2041 to 2044, 2052 to 2057, 2060, 2067 to 2076, 2078, 2080, 2081, 2083, 2091, 2092, 2095 to 2099, 2101 to 2109, 2117, 2118, 2122 to 2131, 2135, 2137, 2138, 2149, 2155, 2172, 2174, 2176, 2186, 2189 to 2192, 2195, 2200, 2201, 2203, 2205, 2212, 2223 to 2263, 2274, 2381, 2389, 2398 to 2400, 2402, 2405 to 2408, 2416, 2421, 2426, 2432 to 2440, 2443, and 2448 of Table I.

Those AMRs which involve a nucleotide sequence that when present in Klebsiella spp. are indicative of resistance to at least one antimicrobial drug are preferably selected from those identified in rows 1587 to 1696, 1743, 1744, 1746 to 1749, 1752 to 1754, 1756 to 1765, 1767 to 1773, 1775 to 1780, 1782, 1792 to 1794, 1796, 1797, 1800, 1803, 1804, 1806, 1807, 1820, 1821, 1829 to 1834, 1836 to 1841, 1843 to 1864, 1866 to 1878, 1880 to 1887, 1889 to 1910, 1912 to 2027, 2034 to 2040, 2045 to 2051, 2058, 2059, 2061 to 2066, 2077, 2079, 2082, 2084 to 2090, 2093, 2094, 2100, 2110 to 2116, 2119 to 2121, 2132, 2133, 2208 to 2211, 2213 to 2222, 2264 to 2273, 2275 to 2380, 2382 to 2388, 2390 to 2396, 2425, 2427, 2431, 2441, 2442, and 2444 to 2446 of Table I. Further, the AMRs which involve at least one difference in at least one nucleotide sequence compared to a reference sequence that when present are indicative of resistance to at least one antimicrobial drug are preferably selected from those identified in rows 1697 to 1717, 1719 to 1736, 1739, 1741, 1742, 1784, 1786, 1788, 1789, 2134, 2136, 2139 to 2148, 2150 to 2154, 2156 to 2171, 2173, 2175, 2177 to 2185, 2187, 2188, 2193, 2194, 2196 to 2199, 2202, 2204, 2206, 2207, 2397 to 2397, 2401, 2403, 2409 to 2415, 2417 to 2420, 2422 to 2424, 2428 to 2430, and 2447 in Table I.

Those AMRs which involve a nucleotide sequence that when absent in Klebsiella spp. are indicative of resistance to at least one antimicrobial drug are preferably selected from those identified in rows 1745, 1750, 1751, 1755, 1766, 1774, 1781, 1795, 1798, 1799, 1801, 1802, 1805, 1808 to 1819, 1822 to 1828, 1835, 1842, 1865, 1879, 1888, 1911, 2028 to 2033, 2041 to 2044, 2052 to 2057, 2060, 2067 to 2076, 2078, 2080, 2081, 2083, 2091, 2092, 2095 to 2099, 2101 to 2109, 2117, 2118, 2122 to 2131, 2212, 2223 to 2263, 2274, 2381, 2389, 2426, 2432 to 2440, and 2443 of Table I. Further, the AMRs which involve at least one difference in at least one nucleotide sequence compared to a reference sequence that when absent are indicative of resistance to at least one antimicrobial drug are preferably selected from those identified in rows 1718, 1737, 1738, 1740, 1783, 1785, 1787, 1790, 1791, 2135, 2137, 2138, 2149, 2155, 2172, 2174, 2176, 2186, 2189 to 2192, 2195, 2200, 2201, 2203, 2205, 2398 to 2400, 2402, 2405 to 2408, 2416, 2421, and 2448 in Table I.

Those AMRs when present in Klebsiella aerogenes which are indicative of resistance to at least one antimicrobial drug are preferably selected from those identified in rows 1587 to 1696, 1697 to 1717, 1719 to 1736, 1739, 1741, and 1742 of Table I. Those AMRs when absent in Klebsiella aerogenes which are indicative of resistance to at least one antimicrobial drug are preferably selected from those identified in rows 1718, 1737, 1738 and 1740 of Table I.

Those AMRs when present in Klebsiella michiganensis which are indicative of resistance to at least one antimicrobial drug are preferably selected from those identified in rows 1743, 1744, 1746 to 1749, 1752 to 1754, 1756 to 1765, 1767 to 1773, 1775 to 1780, 1782, 1784, 1786, 1788, and 1789 of Table I. Those AMRs when absent in Klebsiella michiganensis which are indicative of resistance to at least one antimicrobial drug are preferably selected from those identified in rows 1745, 1750, 1751, 1755, 1766, 1774, 1781, 1783, 1785, 1787, 1790, and 1791 of Table I.

Those AMRs when present in Klebsiella oxytoca which are indicative of resistance to at least one antimicrobial drug are preferably selected from those identified in rows 1792 to 1794, 1796, 1797, 1800, 1803, 1804, 1806, 1807, 1820, 1821, 1829 to 1834, 1836 to 1841, 1843 to 1864, 1866 to 1878, 1880 to 1887, 1889 to 1910, 1912 to 2027, 2034 to 2040, 2045 to 2051, 2058, 2059, 2061 to 2066, 2077, 2079, 2082, 2084 to 2090, 2093, 2094, 2100, 2110 to 2116, 2119 to 2121, 2132, 2133, 2134, 2136, 2139 to 2148, 2150 to 2154, 2156 to 2171, 2173, 2175, 2177 to 2185, 2187, 2188, 2193, 2194, 2196 to 2199, 2202, 2204, 2206, and 2207 of Table I. Those AMRs when absent in Klebsiella oxytoca which are indicative of resistance to at least one antimicrobial drug are preferably selected from those identified in rows 1795, 1798, 1799, 1801, 1802, 1805, 1808 to 1819, 1822 to 1828, 1835, 1842, 1865, 1879, 1888, 1911, 2028 to 2033, 2041 to 2044, 2052 to 2057, 2060, 2067 to 2076, 2078, 2080, 2081, 2083, 2091, 2092, 2095 to 2099, 2101 to 2109, 2117, 2118, 2122 to 2131, 2135, 2137, 2138, 2149, 2155, 2172, 2174, 2176, 2186, 2189 to 2192, 2195, 2200, 2201, 2203, and 2205 of Table I.

Those AMRs when present in Klebsiella pneumoniae which are indicative of resistance to at least one antimicrobial drug are preferably selected from those identified in rows 2208 to 2211, 2213 to 2222, 2264 to 2273, 2275 to 2380, 2382 to 2388, 2390 to 2396, 2397 to 2397, 2401, 2403, 2409 to 2415, 2417 to 2420, and 2422 to 2424 of Table I. Those AMRs when absent in Klebsiella pneumoniae which are indicative of resistance to at least one antimicrobial drug are preferably selected from those identified in rows 2212, 2223 to 2263, 2274, 2381, 2389, 2398 to 2400, 2402, 2405 to 2408, 2416, and 2421 of Table I.

Those AMRs when present in Klebsiella quasipneumoniae which are indicative of resistance to at least one antimicrobial drug are preferably selected from those identified in rows 2425, and 2427 to 2430 of Table I. Those AMRs when absent in Klebsiella quasipneumoniae which are indicative of resistance to at least one antimicrobial drug are preferably selected from those identified in row 2426 of Table I.

Those AMRs when present in Klebsiella variicola which are indicative of resistance to at least one antimicrobial drug are preferably selected from those identified in rows 2431, 2441, 2442, and 2444 to 2447 of Table I. Those AMRs when absent in Klebsiella variicola which are indicative of resistance to at least one antimicrobial drug are preferably selected from those identified in row 2448 of Table I. In embodiments where the Klebsiella microorganism is a particular species of Klebsiella, the methods preferably comprise determining the presence and/or absence of the AMRs determined to be indicative of antimicrobial drug resistance in that species set forth in Table I.

It is intended that the foregoing disclosure regarding the AMRs for Klebsiella spp. be equally applicable to all of the methods disclosed herein, in particular those methods which comprise steps of determining the presence or absence of nucleotide sequences and/or differences in nucleotide sequences. In an embodiment, those AMRs for Klebsiella spp. having p values not greater than 0.1, 0.01, 0.001, 0.0001, 10⁻⁵, 10⁻¹⁰, or 10⁻²⁰ as set forth in Table II are the AMRs used in the methods disclosed herein to indicate antimicrobial drug resistance. In an embodiment, those AMRs for Klebsiella spp. having ppv values greater than 0.7, 0.75, 0.8, 0.85, 0.9, 0.95, or 0.98 as set forth in Table II are the AMRs used in the methods disclosed herein to indicate antimicrobial drug resistance. In an embodiment, those AMRs for Klebsiella spp. having p values not greater than 0.1, 0.01, 0.001, 0.0001, 10⁻⁵, 10⁻¹⁰, or 10⁻²⁰ as set forth in Table II and having ppv values greater than 0.7, 0.75, 0.8, 0.85, 0.9, 0.95, or 0.98 as set forth in Table II are the AMRs used in the methods disclosed herein to indicate antimicrobial drug resistance.

In accordance with the foregoing, in an exemplary embodiment, where the microorganism is Klebsiella, the methods disclosed herein comprise (i) determining the presence and/or absence in Klebsiella spp. of at least one nucleotide sequence identified in rows 1587 to 1696, 1743 to 1782, 1792 to 2133, 2208 to 2396, 2425 to 2427, and 2431 to 2446 of Table I or a variant of the nucleotide sequence, and/or (ii) determining the presence and/or absence in Klebsiella spp., of at least one difference in at least one nucleotide sequence identified in rows 1697 to 1742, 1783 to 1791, 2134 to 2207, 2397 to 2424, 2428 to 2430, 2447, and 2448 of Table I compared with a reference sequence.

In an embodiment, the method comprises determining the presence in Klebsiella spp. of at least one nucleotide sequence identified in rows 1587 to 1696, 1743, 1744, 1746 to 1749, 1752 to 1754, 1756 to 1765, 1767 to 1773, 1775 to 1780, 1782, 1792 to 1794, 1796, 1797, 1800, 1803, 1804, 1806, 1807, 1820, 1821, 1829 to 1834, 1836 to 1841, 1843 to 1864, 1866 to 1878, 1880 to 1887, 1889 to 1910, 1912 to 2027, 2034 to 2040, 2045 to 2051, 2058, 2059, 2061 to 2066, 2077, 2079, 2082, 2084 to 2090, 2093, 2094, 2100, 2110 to 2116, 2119 to 2121, 2132, 2133, 2208 to 2211, 2213 to 2222, 2264 to 2273, 2275 to 2380, 2382 to 2388, 2390 to 2396, 2425, 2427, 2431, 2441, 2442, and 2444 to 2446 of Table I or a variant of the nucleotide sequence. In an embodiment, the method comprises determining the presence in Klebsiella spp. of at least one difference in at least one nucleotide sequence identified in rows 1697 to 1717, 1719 to 1736, 1739, 1741, 1742, 1784, 1786, 1788, 1789, 2134, 2136, 2139 to 2148, 2150 to 2154, 2156 to 2171, 2173, 2175, 2177 to 2185, 2187, 2188, 2193, 2194, 2196 to 2199, 2202, 2204, 2206, 2207, 2397 to 2397, 2401, 2403, 2409 to 2415, 2417 to 2420, 2422 to 2424, 2428 to 2430, and 2447 of Table I compared with a reference sequence.

In an embodiment, the method comprises determining the absence in Klebsiella spp. of at least one nucleotide sequence identified in rows 1745, 1750, 1751, 1755, 1766, 1774, 1781, 1795, 1798, 1799, 1801, 1802, 1805, 1808 to 1819, 1822 to 1828, 1835, 1842, 1865, 1879, 1888, 1911, 2028 to 2033, 2041 to 2044, 2052 to 2057, 2060, 2067 to 2076, 2078, 2080, 2081, 2083, 2091, 2092, 2095 to 2099, 2101 to 2109, 2117, 2118, 2122 to 2131, 2212, 2223 to 2263, 2274, 2381, 2389, 2426, 2432 to 2440, and 2443 of Table I or a variant of the nucleotide sequence. In an embodiment, the method comprises determining the absence in Klebsiella spp. of at least one difference in at least one nucleotide sequence identified in rows 1718, 1737, 1738, 1740, 1783, 1785, 1787, 1790, 1791, 2135, 2137, 2138, 2149, 2155, 2172, 2174, 2176, 2186, 2189 to 2192, 2195, 2200, 2201, 2203, 2205, 2398 to 2400, 2402, 2405 to 2408, 2416, 2421, and 2448 of Table I compared with a reference sequence.

Where the microorganism is Morganella spp., preferably Morganella morganii, the AMRs that when present or absent are indicative of resistance to at least one antimicrobial drug are preferably selected from those identified in 2460 to 2621 of Table I. The AMRs can further comprise those identified in row 3798 of Table I. A preferred difference in the nucleotide sequence identified in rows 2570 to2621 of Table I is set forth in each of the same respective rows of Table I. The preferred at least one antimicrobial drug to which resistance is indicated by the presence of the AMR is disclosed for the same respective marker ID of the AMR in Table II.

Those AMRs when present in Morganella spp. which are indicative of resistance to at least one antimicrobial drug are preferably selected from those identified in rows 2460 to 2471, 2473 to 2488, 2490 to 2495, 2511 to 2515, 2519 to 2523, 2525, 2526, 2528 to 2530, 2539 to 2586, 2588, 2589, 2591 to 2621 of Table I.

Those AMRs when absent in Morganella spp. which are indicative of resistance to at least one antimicrobial drug are preferably selected from those identified in rows 2472, 2489, 2496 to 2510, 2516 to 2518, 2524, 2527, 2531 to 2538, 2587, and 2590 of Table I.

Those AMRs which involve a nucleotide sequence that when present in Morganella spp. are indicative of resistance to at least one antimicrobial drug are preferably selected from those identified in rows 2460 to 2471, 2473 to 2488, 2490 to 2495, 2511 to 2515, 2519 to 2523, 2525, 2526, 2528 to 2530, 2539 to 2569 of Table I. Further, the AMRs which involve at least one difference in at least one nucleotide sequence compared to a reference sequence that when present are indicative of resistance to at least one antimicrobial drug are preferably selected from those identified in rows 2570 to 2586, 2588, 2589, 2591 to 2621 in Table I.

Those AMRs which involve a nucleotide sequence that when absent in Morganella spp. are indicative of resistance to at least one antimicrobial drug are preferably selected from those identified in rows 2472, 2489, 2496 to 2510, 2516 to 2518, 2524, 2527, and 2531 to 2538 of Table I. Further, the AMRs which involve at least one difference in at least one nucleotide sequence compared to a reference sequence that when absent are indicative of resistance to at least one antimicrobial drug are preferably selected from those identified in rows 2587, and 2590 in Table I.

It is intended that the foregoing disclosure regarding the AMRs for Morganella spp. be equally applicable to all of the methods disclosed herein, in particular those methods which comprise steps of determining the presence or absence of nucleotide sequences and/or differences in nucleotide sequences. In an embodiment, those AMRs for Morganella spp. having p values not greater than 0.1, 0.01, 0.001, 0.0001, 10⁻⁵, 10⁻¹⁰, or 10⁻²⁰ as set forth in Table II are the AMRs used in the methods disclosed herein to indicate antimicrobial drug resistance. In an embodiment, those AMRs for Morganella spp. having ppv values greater than 0.7, 0.75, 0.8, 0.85, 0.9, 0.95, or 0.98 as set forth in Table II are the AMRs used in the methods disclosed herein to indicate antimicrobial drug resistance. In an embodiment, those AMRs for Morganella spp. having p values not greater than 0.1, 0.01, 0.001, 0.0001, 10⁻⁵, 10⁻¹⁰, or 10⁻²⁰ as set forth in Table II and having ppv values greater than 0.7, 0.75, 0.8, 0.85, 0.9, 0.95, or 0.98 as set forth in Table II are the AMRs used in the methods disclosed herein to indicate antimicrobial drug resistance.

In accordance with the foregoing, in an exemplary embodiment, where the microorganism is Morganella, the methods disclosed herein comprise (i) determining the presence and/or absence in Morganella spp. of at least one nucleotide sequence identified in rows 2460 to 2569 of Table I or a variant of the nucleotide sequence, and/or (ii) determining the presence and/or absence in Morganella spp., of at least one difference in at least one nucleotide sequence identified in rows 2570 to 2621 of Table I compared with a reference sequence.

In an embodiment, the method comprises determining the presence in Morganella spp. of at least one nucleotide sequence identified in rows 2460 to 2471, 2473 to 2488, 2490 to 2495, 2511 to 2515, 2519 to 2523, 2525, 2526, 2528 to 2530, 2539 to 2569 of Table I or a variant of the nucleotide sequence. In an embodiment, the method comprises determining the presence in Morganella spp. of at least one difference in at least one nucleotide sequence identified in rows 2570 to 2586, 2588, 2589, 2591 to 2621 of Table I compared with a reference sequence.

In an embodiment, the method comprises determining the absence in Morganella spp. of at least one nucleotide sequence identified in rows 2472, 2489, 2496 to 2510, 2516 to 2518, 2524, 2527, and 2531 to 2538 of Table I or a variant of the nucleotide sequence. In an embodiment, the method comprises determining the absence in Morganella spp. of at least one difference in at least one nucleotide sequence identified in rows 2587 and 2590 of Table I compared with a reference sequence.

Where the microorganism is Proteus spp., preferably Proteus mirabilis or Proteus vulgaris, the AMRs that when present or absent are indicative of resistance to at least one antimicrobial drug are preferably selected from those identified in rows 2623 to 2792 of Table I. The AMRs can further comprise those identified in rows 3799 to 3807 of Table I. A preferred difference in the nucleotide sequence identified in rows 2755 to 2788 of Table 1 is set forth in each of the same respective rows of Table I. The preferred at least one antimicrobial drug to which resistance is indicated by the presence or absence of the AMR is disclosed for the same respective marker ID of the AMR in Table II.

Those AMRs when present in Proteus spp. which are indicative of resistance to at least one antimicrobial drug are preferably selected from those identified in rows 2623 to 2636, 2639, 2646 to 2663, and 2665 to 2792 of Table I.

Those AMRs when absent in Proteus spp. which are indicative of resistance to at least one antimicrobial drug are preferably selected from those identified in rows 2637, 2638, 2640 to 2645, and 2664 of Table I.

Those AMRs which involve a nucleotide sequence that when present in Proteus spp. are indicative of resistance to at least one antimicrobial drug are preferably selected from those identified in rows 2623 to 2636, 2639, 2646 to 2754, and 2789 to 2792 of Table I. Further, the AMRs which involve at least one difference in at least one nucleotide sequence compared to a reference sequence that when present are indicative of resistance to at least one antimicrobial drug are preferably selected from those identified in rows 2755 to 2663, and 2665 to 2788 in Table I.

Those AMRs which involve a nucleotide sequence that when absent in Proteus spp. are indicative of resistance to at least one antimicrobial drug are preferably selected from those identified in rows 2637, 2638, and 2640 to 2645 of Table I. Further, the AMRs which involve at least one difference in at least one nucleotide sequence compared to a reference sequence that when absent are indicative of resistance to at least one antimicrobial drug are preferably selected from those identified in row 2664 in Table I.

Those AMRs when present in Proteus mirabilis which are indicative of resistance to at least one antimicrobial drug are preferably selected from those identified in rows 2623 to 2636, 2639, 2646 to 2663, and 2665 to 2788 of Table I. Those AMRs when absent in Proteus mirabilis which are indicative of resistance to at least one antimicrobial drug are preferably selected from those identified in rows 2637, 2638, 2640 to 2645, and 2664 of Table I.

Those AMRs when present in Proteus vulgaris which are indicative of resistance to at least one antimicrobial drug are preferably selected from those identified in rows 2789 to 2792 of Table I.

In embodiments where the Proteus microorganism is a particular species of Proteus, the methods preferably comprise determining the presence and/or absence of the AMRs determined to be indicative of antimicrobial drug resistance in that species set forth in Table I.

It is intended that the foregoing disclosure regarding the AMRs for Proteus spp. be equally applicable to all of the methods disclosed herein, in particular those methods which comprise steps of determining the presence or absence of nucleotide sequences and/or differences in nucleotide sequences. In an embodiment, those AMRs for Proteus spp. having p values not greater than 0.1, 0.01, 0.001, 0.0001, 10⁻⁵, 10⁻¹⁰, or 10⁻²⁰ as set forth in Table II are the AMRs used in the methods disclosed herein to indicate antimicrobial drug resistance. In an embodiment, those AMRs for Proteus spp. having ppv values greater than 0.7, 0.75, 0.8, 0.85, 0.9, 0.95, or 0.98 as set forth in Table II are the AMRs used in the methods disclosed herein to indicate antimicrobial drug resistance. In an embodiment, those AMRs for Proteus spp. having p values not greater than 0.1, 0.01, 0.001, 0.0001, 10⁻⁵, 10⁻¹⁰, or 10⁻²⁰ as set forth in Table II and having ppv values greater than 0.7, 0.75, 0.8, 0.85, 0.9, 0.95, or 0.98 as set forth in Table II are the AMRs used in the methods disclosed herein to indicate antimicrobial drug resistance.

In accordance with the foregoing, in an exemplary embodiment, where the microorganism is Proteus, the methods disclosed herein comprise (i) determining the presence and/or absence in Proteus spp. of at least one nucleotide sequence identified in rows 2623 to 2754 and 2789 to 2792 of Table I or a variant of the nucleotide sequence, and/or (ii) determining the presence and/or absence in Proteus spp., of at least one difference in at least one nucleotide sequence identified in rows 2755 to 2788 of Table I compared with a reference sequence.

In an embodiment, the method comprises determining the presence in Proteus spp. of at least one nucleotide sequence identified in rows 2623 to 2636, 2639, 2646 to 2754, and 2789 to 2792 of Table I or a variant of the nucleotide sequence. In an embodiment, the method comprises determining the presence in Proteus spp. of at least one difference in at least one nucleotide sequence identified in rows 2755 to 2663, and 2665 to 2788 of Table I compared with a reference sequence.

In an embodiment, the method comprises determining the absence in Proteus spp. of at least one nucleotide sequence identified in rows 2637, 2638, and 2640 to 2645 of Table I or a variant of the nucleotide sequence. In an embodiment, the method comprises determining the absence in Proteus spp. of at least one difference in at least one nucleotide sequence identified in row 2664 of Table I compared with a reference sequence.

Where the microorganism is Pseudomonas spp., preferably Pseudomonas aeruginosa, Pseudomonas fluorescens or Pseudomonas nitroreducens, the AMRs that when present or absent are indicative of resistance to at least one antimicrobial drug are preferably selected from those identified in rows 2793 to 2878 of Table I. The AMRs can further comprise those identified in rows 3808 and 3809 of Table I. A preferred difference in the nucleotide sequence identified in rows 2862 to 2876 of Table 1 is set forth in each of the same respective rows of Table I. The preferred at least one antimicrobial drug to which resistance is indicated by the presence or absence of the AMR is disclosed for the same respective marker ID of the AMR in Table II.

Those AMRs when present in Pseudomonas spp. which are indicative of resistance to at least one antimicrobial drug are preferably selected from those identified in rows 2793, 2794, 2796 to 2862, 2864 to 2870, and 2872 to 2878 of Table I. Further, the AMRs which involve at least one difference in at least one nucleotide sequence compared to a reference sequence that when present are indicative of resistance to at least one antimicrobial drug are preferably selected from those identified in rows 2862, 2864 to 2870, and 2872 to 2876 in Table I.

Those AMRs when absent in Pseudomonas spp. which are indicative of resistance to at least one antimicrobial drug are preferably selected from those identified in row 2795 of Table I. Further, the AMRs which involve at least one difference in at least one nucleotide sequence compared to a reference sequence that when absent are indicative of resistance to at least one antimicrobial drug are preferably selected from those identified in rows 2863 and 2871 in Table I.

Those AMRs which involve a nucleotide sequence that when present in Pseudomonas spp. are indicative of resistance to at least one antimicrobial drug are preferably selected from those identified in rows 2793, 2794, 2796 to 2861, 2877, and 2878 of Table I. Further, the AMRs which involve at least one difference in at least one nucleotide sequence compared to a reference sequence that when present are indicative of resistance to at least one antimicrobial drug are preferably selected from those identified in rows 2862, 2864 to 2870, and 2872 to 2876 in Table I.

Those AMRs which involve a nucleotide sequence that when absent in Pseudomonas spp. are indicative of resistance to at least one antimicrobial drug are preferably selected from those identified in row 2795 of Table I. Further, the AMRs which involve at least one difference in at least one nucleotide sequence compared to a reference sequence that when absent are indicative of resistance to at least one antimicrobial drug are preferably selected from those identified in rows 2863 and 2871 in Table I.

Those AMRs when present in Pseudomonas aeruginosa which are indicative of resistance to at least one antimicrobial drug are preferably selected from those identified in rows 2793, 2794, 2796 to 2862, 2864 to 2870, and 2872 to 2876 of Table I. Those AMRs when absent in Pseudomonas aeruginosa which are indicative of resistance to at least one antimicrobial drug are preferably selected from those identified in rows 2795, 2863, and 2871 of Table I.

Those AMRs when present in Pseudomonas fluorescens which are indicative of resistance to at least one antimicrobial drug are preferably selected from those identified in row 2877 of Table I.

Those AMRs when present in Pseudomonas nitroreducens which are indicative of resistance to at least one antimicrobial drug are preferably selected from those identified in row 2878 of Table I.

In embodiments where the Pseudomonas microorganism is a particular species of Pseudomonas, the methods preferably comprise determining the presence and/or absence of the AMRs determined to be indicative of antimicrobial drug resistance in that species set forth in Table I.

It is intended that the foregoing disclosure regarding the AMRs for Pseudomonas spp. be equally applicable to all of the methods disclosed herein, in particular those methods which comprise steps of determining the presence or absence of nucleotide sequences and/or differences in nucleotide sequences. In an embodiment, those AMRs for Pseudomonas spp. having p values not greater than 0.1, 0.01, 0.001, 0.0001, 10⁻⁵, 10⁻¹⁰, or 10⁻²⁰ as set forth in Table II are the AMRs used in the methods disclosed herein to indicate antimicrobial drug resistance. In an embodiment, those AMRs for Pseudomonas spp. having ppv values greater than 0.7, 0.75, 0.8, 0.85, 0.9, 0.95, or 0.98 as set forth in Table II are the AMRs used in the methods disclosed herein to indicate antimicrobial drug resistance. In an embodiment, those AMRs for Pseudomonas spp. having p values not greater than 0.1, 0.01, 0.001, 0.0001, 10⁻⁵, 10⁻¹⁰, or 10⁻²⁰ as set forth in Table II and having ppv values greater than 0.7, 0.75, 0.8, 0.85, 0.9, 0.95, or 0.98 as set forth in Table II are the AMRs used in the methods disclosed herein to indicate antimicrobial drug resistance.

In accordance with the foregoing, in an exemplary embodiment, where the microorganism is Pseudomonas, the methods disclosed herein comprise (i) determining the presence and/or absence in Pseudomonas spp. of at least one nucleotide sequence identified in rows 2793 to 2861, 2877, and 2878 of Table I or a variant of the nucleotide sequence, and/or (ii) determining the presence and/or absence in Pseudomonas spp., of at least one difference in at least one nucleotide sequence identified in rows 2862 to 2876 of Table I compared with a reference sequence.

In an embodiment, the method comprises determining the presence in Pseudomonas spp. of at least one nucleotide sequence identified in rows 2793, 2794, 2796 to 2861, 2877, and 2878 of Table I or a variant of the nucleotide sequence. In an embodiment, the method comprises determining the presence in Pseudomonas spp. of at least one difference in at least one nucleotide sequence identified in rows 2862, 2864 to 2870, and 2872 to 2876 of Table I compared with a reference sequence.

In an embodiment, the method comprises determining the absence in Pseudomonas spp. of at least one nucleotide sequence identified in row 2795 of Table I or a variant of the nucleotide sequence. In an embodiment, the method comprises determining the absence in Pseudomonas spp. of at least one difference in at least one nucleotide sequence identified in rows 2863 and 2871 of Table I compared with a reference sequence.

Where the microorganism is Salmonella spp., preferably Salmonella enterica, the AMRs that when present or absent are indicative of resistance to at least one antimicrobial drug are preferably selected from those identified in rows 2885 to 3008 of Table I. The AMRs can further comprise those identified in rows 3810 to 3815 of Table I. A preferred difference in the nucleotide sequence identified in rows 2991 to 3008 of Table 1 is set forth in each of the same respective rows of Table I. The preferred at least one antimicrobial drug to which resistance is indicated by the presence of the AMR is disclosed for the same respective marker ID of the AMR in Table II.

Those AMRs when present in Salmonella spp. which are indicative of resistance to at least one antimicrobial drug are preferably selected from those identified in rows 2885 to 2898, and 2990 to 3008 of Table I.

Those AMRs when absent in Salmonella spp. which are indicative of resistance to at least one antimicrobial drug are preferably selected from those identified in row 2899 of Table I.

Those AMRs which involve a nucleotide sequence that when present in Salmonella spp. are indicative of resistance to at least one antimicrobial drug are preferably selected from those identified in rows 2885 to 2898, and 2990 of Table I. Further, the AMRs which involve at least one difference in at least one nucleotide sequence compared to a reference sequence that when present are indicative of resistance to at least one antimicrobial drug are preferably selected from those identified in rows 2991 to 3008 in Table I.

Those AMRs which involve a nucleotide sequence that when absent in Salmonella spp. are indicative of resistance to at least one antimicrobial drug are preferably selected from those identified in row 2899 of Table I.

It is intended that the foregoing disclosure regarding the AMRs for Salmonella spp. be equally applicable to all of the methods disclosed herein, in particular those methods which comprise steps of determining the presence or absence of nucleotide sequences and/or differences in nucleotide sequences. In an embodiment, those AMRs for Salmonella spp. having p values not greater than 0.1, 0.01, 0.001, 0.0001, 10⁻⁵, 10⁻¹⁰, or 10⁻²⁰ as set forth in Table II are the AMRs used in the methods disclosed herein to indicate antimicrobial drug resistance. In an embodiment, those AMRs for Salmonella spp. having ppv values greater than 0.7, 0.75, 0.8, 0.85, 0.9, 0.95, or 0.98 as set forth in Table II are the AMRs used in the methods disclosed herein to indicate antimicrobial drug resistance. In an embodiment, those AMRs for Salmonella spp. having p values not greater than 0.1, 0.01, 0.001, 0.0001, 10⁻⁵, 10⁻¹⁰, or 10⁻²⁰ as set forth in Table II and having ppv values greater than 0.7, 0.75, 0.8, 0.85, 0.9, 0.95, or 0.98 as set forth in Table II are the AMRs used in the methods disclosed herein to indicate antimicrobial drug resistance.

In accordance with the foregoing, in an exemplary embodiment, where the microorganism is Salmonella, the methods disclosed herein comprise (i) determining the presence in Salmonella spp. of at least one nucleotide sequence identified in rows 2885 to 2990 of Table I or a variant of the nucleotide sequence, and/or (ii) determining the presence in Salmonella spp., of at least one difference in at least one nucleotide sequence identified in rows 2991 to 3008 of Table I compared with a reference sequence.

In an embodiment, the method comprises determining the presence in Salmonella spp. of at least one nucleotide sequence identified in rows 2885 to 2898, and 2990 of Table I or a variant of the nucleotide sequence. In an embodiment, the method comprises determining the presence in Salmonella spp. of at least one difference in at least one nucleotide sequence identified in rows 2991 to 3008 of Table I compared with a reference sequence.

In an embodiment, the method comprises determining the absence in Salmonella spp. of at least one nucleotide sequence identified in row 2899 of Table I or a variant of the nucleotide sequence.

Where the microorganism is Serratia spp., preferably Serratia ficaria, Serratia fonticola, Serratia liquefaciens, Serratia marcescens Serratia odorifera, Serratia plymuthica or Serratia rubidaea, the AMRs that when present or absent are indicative of resistance to at least one antimicrobial drug are preferably selected from those identified in rows 3009 to 3200 of Table I. The AMRs can further comprise those identified in row 3816 of Table I. A preferred difference in the nucleotide sequence identified in rows 3014 to 3022, 3029, 3030, 3132 to 3185, 3186, 3187, 3199, and 3200 of Table I is set forth in each of the same respective rows of Table I. The preferred at least one antimicrobial drug to which resistance is indicated by the presence or absence of the AMR is disclosed for the same respective marker ID of the AMR in Table II.

Those AMRs when present in Serratia spp. which are indicative of resistance to at least one antimicrobial drug are preferably selected from those identified in rows of 3012, 3014, 3021, 3023, 3024, 3028, 3030 to 3043, 3046 to 3112, 3114 to 3135, 3137 to 3146, 3149 to 3185, 3188 to 3190, 3196, and 3198 to 3200 Table I.

Those AMRs when absent in Serratia spp. which are indicative of resistance to at least one antimicrobial drug are preferably selected from those identified in rows 3009 to 3011, 3013, 3015 to 3020, 3022, 3025 to 3027, 3029, 3044, 3045, 3113, 3136, 3147, 3148, 3186, 3187, 3191 to 3195, and 3197 of Table I.

Those AMRs which involve a nucleotide sequence that when present in Serratia spp. are indicative of resistance to at least one antimicrobial drug are preferably selected from those identified in rows 3012, 3023, 3024, 3028, 3031 to 3043, 3046 to 3112, 3114 to 3131, 3188 to 3190, 3196, and 3198 of Table I. Further, the AMRs which involve at least one difference in at least one nucleotide sequence compared to a reference sequence that when present are indicative of resistance to at least one antimicrobial drug are preferably selected from those identified in rows 3014, 3021, 3030, 3132 to 3135, 3137 to 3147, 3149 to 3185, 3199, and 3200 in Table I.

Those AMRs which involve a nucleotide sequence that when absent in Serratia spp. are indicative of resistance to at least one antimicrobial drug are preferably selected from those identified in rows 3009 to 3011, 3013, 3025 to 3027, 3044, 3045, 3113, 3191 to 3195, and 3197 of Table I. Further, the AMRs which involve at least one difference in at least one nucleotide sequence compared to a reference sequence that when absent are indicative of resistance to at least one antimicrobial drug are preferably selected from those identified in rows 3015 to 3020, 3022, 3029, 3136, 3147, 3148, 3186, and 3187 in Table I.

Those AMRs when absent in Serratia ficaria which are indicative of resistance to at least one antimicrobial drug are preferably selected from those identified in row 3009 of Table I.

Those AMRs when present in Serratia fonticola which are indicative of resistance to at least one antimicrobial drug are preferably selected from those identified in rows 3012, 3014, and 3021 of Table I. Those AMRs when absent in Serratia fonticola which are indicative of resistance to at least one antimicrobial drug are preferably selected from those identified in rows 3010, 3011, 3013, 3015 to 3020, and 3022 of Table I.

Those AMRs when present in Serratia liquefaciens which are indicative of resistance to at least one antimicrobial drug are preferably selected from those identified in rows 3023, 3024, 3028, and 3030 of Table I. Those AMRs when absent in Serratia liquefaciens which are indicative of resistance to at least one antimicrobial drug are preferably selected from those identified in rows 3025 to 3027, and 3029 of Table I.

Those AMRs when present in Serratia marcescens which are indicative of resistance to at least one antimicrobial drug are preferably selected from those identified in rows 3031 to 3043, 3046 to 3112, 3114 to 3135, 3137 to 3146, and 3149 to 3185 of Table I. Those AMRs when absent in Serratia marcescens which are indicative of resistance to at least one antimicrobial drug are preferably selected from those identified in rows 3044, 3045, 3113, 3136, 3147, and 3148 of Table I.

Those AMRs when absent in Serratia odorifera which are indicative of resistance to at least one antimicrobial drug are preferably selected from those identified in rows 3186 and 3187 of Table I.

Those AMRs when present in Serratia plymuthica which are indicative of resistance to at least one antimicrobial drug are preferably selected from those identified in rows 3188 and 3189 of Table I.

Those AMRs when present in Serratia rubidaea which are indicative of resistance to at least one antimicrobial drug are preferably selected from those identified in rows 3190, 3196, 3198 to 3200 of Table I. Those AMRs when absent in Serratia rubidaea which are indicative of resistance to at least one antimicrobial drug are preferably selected from those identified in rows 3191 to 3195, and 3197 of Table I.

In embodiments where the Serratia microorganism is a particular species of Serratia, the methods preferably comprise determining the presence and/or absence of the AMRs determined to be indicative of antimicrobial drug resistance in that species set forth in Table I.

It is intended that the foregoing disclosure regarding the AMRs for Serratia spp. be equally applicable to all of the methods disclosed herein, in particular those methods which comprise steps of determining the presence or absence of nucleotide sequences and/or differences in nucleotide sequences. In an embodiment, those AMRs for Serratia spp. having p values not greater than 0.1, 0.01, 0.001, 0.0001, 10⁻⁵, 10⁻¹⁰, or 10⁻²⁰ as set forth in Table II are the AMRs used in the methods disclosed herein to indicate antimicrobial drug resistance. In an embodiment, those AMRs for Serratia spp. having ppv values greater than 0.7, 0.75, 0.8, 0.85, 0.9, 0.95, or 0.98 as set forth in Table II are the AMRs used in the methods disclosed herein to indicate antimicrobial drug resistance. In an embodiment, those AMRs for Serratia spp. having p values not greater than 0.1, 0.01, 0.001, 0.0001, 10⁻⁵, 10⁻¹⁰, or 10⁻²⁰ as set forth in Table II and having ppv values greater than 0.7, 0.75, 0.8, 0.85, 0.9, 0.95, or 0.98 as set forth in Table II are the AMRs used in the methods disclosed herein to indicate antimicrobial drug resistance.

In accordance with the foregoing, in an exemplary embodiment, where the microorganism is Serratia, the methods disclosed herein comprise (i) determining the presence and/or absence in Serratia spp. of at least one nucleotide sequence identified in rows 3009 to 3013, 3023 to 3028, 3031 to 3131, 3188, and 3189 to 3198 of Table I or a variant of the nucleotide sequence, and/or (ii) determining the presence and/or absence in Serratia spp., of at least one difference in at least one nucleotide sequence identified in rows 3014 to 3022, 3029, 3030, 3132 to 3185, 3186, 3187, 3199, and 3200 of Table I compared with a reference sequence.

In an embodiment, the method comprises determining the presence in Serratia spp. of at least one nucleotide sequence identified in rows 3012, 3023, 3024, 3028, 3031 to 3043, 3046 to 3112, 3114 to 3131, 3188 to 3190, 3196, and 3198 of Table I or a variant of the nucleotide sequence. In an embodiment, the method comprises determining the presence in Serratia spp. of at least one difference in at least one nucleotide sequence identified in rows 3014, 3021, 3030, 3132 to 3135, 3137 to 3147, 3149 to 3185, 3199, and 3200 of Table I compared with a reference sequence.

In an embodiment, the method comprises determining the absence in Serratia spp. of at least one nucleotide sequence identified in rows 3009 to 3011, 3013, 3025 to 3027, 3044, 3045, 3113, 3191 to 3195, and 3197 of Table I or a variant of the nucleotide sequence. In an embodiment, the method comprises determining the absence in Serratia spp. of at least one difference in at least one nucleotide sequence identified in rows 3015 to 3020, 3022, 3029, 3136, 3147, 3148, 3186, and 3187 of Table I compared with a reference sequence.

Where the microorganism is Stenotrophomonas spp., preferably Stenotrophomonas maltophilia, the AMRs that when present are indicative of resistance to at least one antimicrobial drug are preferably selected from those identified in rows 3746 to 3751 of Table I. The AMRs can further comprise those identified in row 3840 of Table I. The preferred at least one antimicrobial drug to which resistance is indicated by the presence of the AMR is disclosed for the same respective marker ID of the AMR in Table II.

It is intended that the foregoing disclosure regarding the AMRs for Stenotrophomonas spp. be equally applicable to all of the methods disclosed herein, in particular those methods which comprise steps of determining the presence or absence of nucleotide sequences and/or differences in nucleotide sequences. In an embodiment, those AMRs for Stenotrophomonas spp. having p values not greater than 0.1, 0.01, 0.001, 0.0001, 10⁻⁵, 10⁻¹⁰, or 10⁻²⁰ as set forth in Table II are the AMRs used in the methods disclosed herein to indicate antimicrobial drug resistance. In an embodiment, those AMRs for Stenotrophomonas spp. having ppv values greater than 0.7, 0.75, 0.8, 0.85, 0.9, 0.95, or 0.98 as set forth in Table II are the AMRs used in the methods disclosed herein to indicate antimicrobial drug resistance. In an embodiment, those AMRs for Stenotrophomonas spp. having p values not greater than 0.1, 0.01, 0.001, 0.0001, 10⁻⁵, 10⁻¹⁰, or 10⁻²⁰ as set forth in Table II and having ppv values greater than 0.7, 0.75, 0.8, 0.85, 0.9, 0.95, or 0.98 as set forth in Table II are the AMRs used in the methods disclosed herein to indicate antimicrobial drug resistance.

In accordance with the foregoing, in an exemplary embodiment, where the microorganism is Stenotrophomonas, the methods disclosed herein comprise (i) determining the presence in Stenotrophomonas spp. of at least one nucleotide sequence identified in rows 3746 to 3751 of Table I or a variant of the nucleotide sequence.

Where the microorganism is Kluyvera spp., preferably Kluyvera intermedia, the AMRs that when present are indicative of resistance to at least one antimicrobial drug are preferably selected from those identified in rows 2449 to 2459 of Table I. The preferred at least one antimicrobial drug to which resistance is indicated by the presence of the AMR is disclosed for the same respective marker ID of the AMR in Table II.

Those AMRs when present in Kluyvera spp. which are indicative of resistance to at least one antimicrobial drug are preferably selected from those identified in rows 2449 to 2458 of Table I. Those AMRs when absent in Proteus spp. which are indicative of resistance to at least one antimicrobial drug are preferably selected from those identified in row 2459 of Table I.

Those AMRs which involve a nucleotide sequence that when present in Kluyvera spp. are indicative of resistance to at least one antimicrobial drug are preferably selected from those identified in rows 2449 to 2457 of Table I. Further, the AMRs which involve at least one difference in at least one nucleotide sequence compared to a reference sequence that when present are indicative of resistance to at least one antimicrobial drug are preferably selected from those identified in row 2458 in Table I.

Further, the AMRs which involve at least one difference in at least one nucleotide sequence compared to a reference sequence that when absent are indicative of resistance to at least one antimicrobial drug are preferably selected from those identified in row 2459 in Table I.

It is intended that the foregoing disclosure regarding the AMRs for Kluyvera spp. be equally applicable to all of the methods disclosed herein, in particular those methods which comprise steps of determining the presence or absence of nucleotide sequences and/or differences in nucleotide sequences. In an embodiment, those AMRs for Kluyvera spp. having p values not greater than 0.1, 0.01, 0.001, 0.0001, 10⁻⁵, 10⁻¹⁰, or 10⁻²⁰ as set forth in Table II are the AMRs used in the methods disclosed herein to indicate antimicrobial drug resistance. In an embodiment, those AMRs for Kluyvera spp. having ppv values greater than 0.7, 0.75, 0.8, 0.85, 0.9, 0.95, or 0.98 as set forth in Table II are the AMRs used in the methods disclosed herein to indicate antimicrobial drug resistance. In an embodiment, those AMRs for Kluyvera spp. having p values not greater than 0.1, 0.01, 0.001, 0.0001, 10⁻⁵, 10⁻¹⁰, or 10⁻²⁰ as set forth in Table II and having ppv values greater than 0.7, 0.75, 0.8, 0.85, 0.9, 0.95, or 0.98 as set forth in Table II are the AMRs used in the methods disclosed herein to indicate antimicrobial drug resistance.

In accordance with the foregoing, in an exemplary embodiment, where the microorganism is Kluyvera, the methods disclosed herein comprise (i) determining the presence in Kluyvera spp. of at least one nucleotide sequence identified in rows 2449 to 2457 of Table I or a variant of the nucleotide sequence, and/or (ii) determining the presence and/or absence in Kluyvera spp., of at least one difference in at least one nucleotide sequence identified in rows 2458 and 2459 of Table I compared with a reference sequence.

Where the microorganism is Burkholdia spp., preferably Burkholdia cenocepacia, the AMRs that when present are indicative of resistance to at least one antimicrobial drug are preferably selected from those identified in row 331 of Table I. The preferred at least one antimicrobial drug to which resistance is indicated by the presence of the AMR is disclosed for the same respective marker ID of the AMR in Table II.

In accordance with the foregoing, in an exemplary embodiment, where the microorganism is Burkholdia, the methods disclosed herein comprise (i) determining the presence in Burkholdia spp. of the nucleotide sequence identified in row 331 of Table I or a variant of the nucleotide sequence.

Where the microorganism is Pantoea spp., preferably Pantoea vagans, the AMRs that when present are indicative of resistance to at least one antimicrobial drug are preferably selected from those identified in row 2622 of Table I. The preferred at least one antimicrobial drug to which resistance is indicated by the presence of the AMR is disclosed for the same respective marker ID of the AMR in Table II.

In accordance with the foregoing, in an exemplary embodiment, where the microorganism is Pantoea, the methods disclosed herein comprise (i) determining the presence in Pantoea spp. of the nucleotide sequence identified in row 2622 of Table I or a variant of the nucleotide sequence.

Where the microorganism is Shigella spp., preferably Shigella boydii, Shigella dysenteriae, Shigella flexneri, or Shigella sonnei, the AMRs that when present or absent are indicative of resistance to at least one antimicrobial drug are preferably selected from those identified in rows 3201 to 3375 of Table I. The AMRS can further comprise those identified in rows 3817 to 3823 of Table I. A preferred difference in the nucleotide sequence identified in rows 3235, 3236, 3257 to 3262, and 3353 to 3375 of Table I is set forth in each of the same respective rows of Table I. The preferred at least one antimicrobial drug to which resistance is indicated by the presence or absence of the AMR is disclosed for the same respective marker ID of the AMR in Table II.

Those AMRs when present in Shigella spp. which are indicative of resistance to at least one antimicrobial drug are preferably selected from those identified in rows 3201, 3203 to 3223, 3225 to 3236, 3238 to 3262, 3264 to 3280, 3282 to 3285, 3287, 3289 to 3355, 3357 to 3373, and 3375 of Table I.

Those AMRs when absent in Shigella spp. which are indicative of resistance to at least one antimicrobial drug are preferably selected from those identified in rows 3202, 3224, 3237, 3263, 3281, 3286, 3288, 3356, and 3374 of Table I.

Those AMRs which involve a nucleotide sequence that when present in Shigella spp. are indicative of resistance to at least one antimicrobial drug are preferably selected from those identified in rows 3201, 3203 to 3223, 3225 to 3234, 3238 to 3256, 3264 to 3280, 3282 to 3285, 3287, and 3289 to 3352 of Table I. Further, the AMRs which involve at least one difference in at least one nucleotide sequence compared to a reference sequence that when present are indicative of resistance to at least one antimicrobial drug are preferably selected from those identified in rows 3235, 3236, 3257 to 3262, 3353 to 3355, 3357 to 3373, and 3375 in Table I.

Those AMRs which involve a nucleotide sequence that when absent in Shigella spp. are indicative of resistance to at least one antimicrobial drug are preferably selected from those identified in rows 3202, 3224, 3237, 3263, 3281, 3286, and 3288 of Table I. Further, the AMRs which involve at least one difference in at least one nucleotide sequence compared to a reference sequence that when absent are indicative of resistance to at least one antimicrobial drug are preferably selected from those identified in rows 3356 and 3374 in Table I.

Those AMRs when present in Shigella boydii which are indicative of resistance to at least one antimicrobial drug are preferably selected from those identified in rows 3201, 3203 to 3223, and 3225 to 3236 of Table I. Those AMRs when absent in Shigella boydii which are indicative of resistance to at least one antimicrobial drug are preferably selected from those identified in row 3202 and 3224 of Table I.

Those AMRs when absent in Shigella dysenteriae which are indicative of resistance to at least one antimicrobial drug are preferably selected from those identified in row 3237 of Table I.

Those AMRs when present in Shigella flexneri which are indicative of resistance to at least one antimicrobial drug are preferably selected from those identified in rows 3238 to 3262 of Table I.

Those AMRs when present in Shigella sonnei which are indicative of resistance to at least one antimicrobial drug are preferably selected from those identified in rows 3264 to 3280, 3282 to 3285, 3287, 3289 to 3355, 3357 to 3373, and 3375 of Table I. Those AMRs when absent in Shigella sonnei which are indicative of resistance to at least one antimicrobial drug are preferably selected from those identified in rows 3263, 3281, 3286, 3288, 3356, and 3374 of Table I.

In embodiments where the Shigella microorganism is a particular species of Shigella, the methods preferably comprise determining the presence and/or absence of the AMRs determined to be indicative of antimicrobial drug resistance in that species set forth in Table I.

It is intended that the foregoing disclosure regarding the AMRs for Shigella spp. be equally applicable to all of the methods disclosed herein, in particular those methods which comprise steps of determining the presence or absence of nucleotide sequences and/or differences in nucleotide sequences. In an embodiment, those AMRs for Shigella spp. having p values not greater than 0.1, 0.01, 0.001, 0.0001, 10⁻⁵, 10⁻¹⁰, or 10⁻²⁰ as set forth in Table II are the AMRs used in the methods disclosed herein to indicate antimicrobial drug resistance. In an embodiment, those AMRs for Shigella spp. having ppv values greater than 0.7, 0.75, 0.8, 0.85, 0.9, 0.95, or 0.98 as set forth in Table II are the AMRs used in the methods disclosed herein to indicate antimicrobial drug resistance. In an embodiment, those AMRs for Shigella spp. having p values not greater than 0.1, 0.01, 0.001, 0.0001, 10⁻⁵, 10⁻¹⁰, or 10⁻²⁰ as set forth in Table II and having ppv values greater than 0.7, 0.75, 0.8, 0.85, 0.9, 0.95, or 0.98 as set forth in Table II are the AMRs used in the methods disclosed herein to indicate antimicrobial drug resistance.

In accordance with the foregoing, in an exemplary embodiment, where the microorganism is Shigella, the methods disclosed herein comprise (i) determining the presence and/or absence in Shigella spp. of at least one nucleotide sequence identified in rows 3201 to 3234, 3237 to 3256, and 3263 to 3352 of Table I or a variant of the nucleotide sequence, and/or (ii) determining the presence in Shigella spp., of at least one difference in at least one nucleotide sequence identified in rows 3235, 3236, 3257 to 3262, and 3353 to 3375 of Table I compared with a reference sequence.

In an embodiment, the method comprises determining the presence in Shigella spp. of at least one nucleotide sequence identified in rows 3201, 3203 to 3223, 3225 to 3234, 3238 to 3256, 3264 to 3280, 3282 to 3285, 3287, and 3289 to 3352 of Table I or a variant of the nucleotide sequence. In an embodiment, the method comprises determining the presence in Shigella spp. of at least one difference in at least one nucleotide sequence identified in rows 3235, 3236, 3257 to 3262, 3353 to 3355, 3357 to 3373, and 3375 of Table I compared with a reference sequence.

In an embodiment, the method comprises determining the absence in Shigella spp. of at least one nucleotide sequence identified in rows 3202, 3224, 3237, 3263, 3281, 3286, and 3288 of Table I or a variant of the nucleotide sequence. In an embodiment, the method comprises determining the absence in Shigella spp. of at least one difference in at least one nucleotide sequence identified in rows 3356 and 3374 of Table I compared with a reference sequence.

Where the microorganism is Enterobacter spp., preferably Enterobacter asburiae, Enterobacter cancerogenus, Enterobacter cloacae, Enterobacter cloacae complex, Enterobacter hormaechei, Enterobacter ludwigii, Enterobacter roggenkampii, Enterobacter sp. MGH 1, and Enterobacter sp. MGH 15, the AMRs that when present or absent are indicative of resistance to at least one antimicrobial drug are preferably selected from those identified in rows 692 to 1247 of Table I. The AMRs can further comprise those identified in rows 3768 to 3777 of Table I. A preferred difference in the nucleotide sequence identified in rows 698 to 701, 1075 to 1189, 1202 to 1212, 1227 to 1230, 1324, and 1240 to 1245 of Table I is set forth in each of the same respective rows of Table I. The preferred at least one antimicrobial drug to which resistance is indicated by the presence of the AMR is disclosed for the same respective marker ID of the AMR in Table II.

Those AMRs when present in Enterobacter spp. which are indicative of resistance to at least one antimicrobial drug are preferably selected from those identified in rows 692 to 700, 702 to 717, 719 to 988, 991 to 1033, 1035 to 1043, 1047 to 1050, 1052 to 1097, 1099 to 1103, 1105, 1106, 1108 to 1195, 1197 to 1214, 1216 to 1220, 1222, 1226, 1227, 1229 to 1232, 1234, 1238 to 1240, and 1242 to 1246 of Table I.

Those AMRs when absent in Enterobacter spp. which are indicative of resistance to at least one antimicrobial drug are preferably selected from those identified in rows 701, 718, 989, 990, 1034, 1044 to 1046, 1051, 1098, 1104, 1107, 1196, 1215, 1221, 1223 to 1225, 1228, 1233, 1235 to 1237, 1241, and 1247 of Table I.

Those AMRs which involve a nucleotide sequence that when present in Enterobacter spp. are indicative of resistance to at least one antimicrobial drug are preferably selected from those identified in rows 692 to 697, 702 to 717, 719 to 988, 991 to 1033, 1035 to 1043, 1047 to 1050, 1052 to 1074, 1190 to 1195, 1197 to 1201, 1213, 1214, 1216 to 1220, 1222, 1226, 1231, 1232, 1238, 1239, and 1246 of Table I. Further, the AMRs which involve at least one difference in at least one nucleotide sequence compared to a reference sequence that when present are indicative of resistance to at least one antimicrobial drug are preferably selected from those identified in rows 698 to 700, 1075 to 1096, 1099 to 1103, 1105, 1106, 1108 to 1189, 1202 to 1212, 1227, 1229, 1230, 1234, 1240, and 1242 to 1245 in Table I.

Those AMRs which involve a nucleotide sequence that when absent in Enterobacter spp. are indicative of resistance to at least one antimicrobial drug are preferably selected from those identified in rows 718, 989, 990, 1034, 1044 to 1046, 1051, 1196, 1215, 1221, 1223 to 1225, 1233, 1235 to 1237, and 1247 of Table I. Further, the AMRs which involve at least one difference in at least one nucleotide sequence compared to a reference sequence that when absent are indicative of resistance to at least one antimicrobial drug are preferably selected from those identified in rows 701, 1098, 1104, 1107, 1228, and 1241 in Table I.

Those AMRs when present in Enterobacter asburiae which are indicative of resistance to at least one antimicrobial drug are preferably selected from those identified in rows 692 to 699 of Table I.

Those AMRs when present in Enterobacter cancerogenus which are indicative of resistance to at least one antimicrobial drug are preferably selected from those identified in row 700 of Table I. Those AMRs when absent in Enterobacter cancerogenus which are indicative of resistance to at least one antimicrobial drug are preferably selected from those identified in row 701 of Table I.

Those AMRs when present in Enterobacter cloacae which are indicative of resistance to at least one antimicrobial drug are preferably selected from those identified in rows 702 to 717, 719 to 988, 991 to 1033, 1035 to 1043, 1047 to 1050, 1052 to 1097, 1099 to 1103, 1105, 1106, and 1108 to 1189 of Table I. Those AMRs when absent in Enterobacter cloacae which are indicative of resistance to at least one antimicrobial drug are preferably selected from those identified in rows 718, 989, 990, 1034, 1044 to 1046, 1051, 1098, 1104, and 1107 of Table I.

Those AMRs when present in Enterobacter cloacae complex which are indicative of resistance to at least one antimicrobial drug are preferably selected from those identified in rows 1190 to 1195, 1197 to 1212 of Table I. Those AMRs when absent in Enterobacter cloacae complex which are indicative of resistance to at least one antimicrobial drug are preferably selected from those identified in row 1196 of Table I.

Those AMRs when present in Enterobacter hormaechei which are indicative of resistance to at least one antimicrobial drug are preferably selected from those identified in rows 1213, 1214, 1216 to 1220, 1222, 1226, 1227, 1229, 1230 of Table I. Those AMRs when absent in Enterobacter hormaechei which are indicative of resistance to at least one antimicrobial drug are preferably selected from those identified in rows 1215, 1221, 1223 to 1225, and 1228 of Table I.

Those AMRs when present in Enterobacter ludwigii which are indicative of resistance to at least one antimicrobial drug are preferably selected from those identified in rows 1231, 1232, and 1234 of Table I. Those AMRs when absent in Enterobacter lugwigii which are indicative of resistance to at least one antimicrobial drug are preferably selected from those identified in row 1234 of Table I.

Those AMRs when present in Enterobacter roggenkampii which are indicative of resistance to at least one antimicrobial drug are preferably selected from those identified in rows 1238 to 1240, 1242 to 1245 of Table I. Those AMRs when absent in Enterobacter roggenkampii which are indicative of resistance to at least one antimicrobial drug are preferably selected from those identified in rows 1235 to 1237, and 1241 of Table I.

Those AMRs when present in Enterobacter sp. MGH 1 which are indicative of resistance to at least one antimicrobial drug are preferably selected from those identified in row 1246 of Table I.

Those AMRs when absent in Enterobacter sp. MGH 15 which are indicative of resistance to at least one antimicrobial drug are preferably selected from those identified in row 1247 of Table I.

In embodiments where the Enterobacter microorganism is a particular species of Enterobacter, the methods preferably comprise determining the presence and/or absence of the AMRs determined to be indicative of antimicrobial drug resistance in that species set forth in Table I.

It is intended that the foregoing disclosure regarding the AMRs for Enterobacter spp. be equally applicable to all of the methods disclosed herein, in particular those methods which comprise steps of determining the presence or absence of nucleotide sequences and/or differences in nucleotide sequences. In an embodiment, those AMRs for Enterobacter spp. having p values not greater than 0.1, 0.01, 0.001, 0.0001, 10⁻⁵, 10⁻¹⁰, or 10⁻²⁰ as set forth in Table II are the AMRs used in the methods disclosed herein to indicate antimicrobial drug resistance. In an embodiment, those AMRs for Enterobacter spp. having ppv values greater than 0.7, 0.75, 0.8, 0.85, 0.9, 0.95, or 0.98 as set forth in Table II are the AMRs used in the methods disclosed herein to indicate antimicrobial drug resistance. In an embodiment, those AMRs for Enterobacter spp. having p values not greater than 0.1, 0.01, 0.001, 0.0001, 10⁻⁵, 10⁻¹⁰, or 10⁻²⁰ as set forth in Table II and having ppv values greater than 0.7, 0.75, 0.8, 0.85, 0.9, 0.95, or 0.98 as set forth in Table II are the AMRs used in the methods disclosed herein to indicate antimicrobial drug resistance.

In accordance with the foregoing, in an exemplary embodiment, where the microorganism is Enterobacter, the methods disclosed herein comprise (i) determining the presence and/or absence in Enterobacter spp. of at least one nucleotide sequence identified in rows 692 to 697, 702 to 1074, 1190 to 1201, 1213 to 1226, 1231 to 1233, 1235 to 1239, 1246, and 1247 of Table I or a variant of the nucleotide sequence, and/or (ii) determining the presence and/or absence in Enterobacter spp., of at least one difference in at least one nucleotide sequence identified in rows 698 to 701, 1075 to 1189, 1202 to 1212, 1227 to 1230, 1234, and 1240 to 1245 of Table I compared with a reference sequence.

In an embodiment, the method comprises determining the presence in Enterobacter spp. of at least one nucleotide sequence identified in rows 692 to 697, 702 to 717, 719 to 988, 991 to 1033, 1035 to 1043, 1047 to 1050, 1052 to 1074, 1190 to 1195, 1197 to 1201, 1213, 1214, 1216 to 1220, 1222, 1226, 1231, 1232, 1238, 1239, and 1246 of Table I or a variant of the nucleotide sequence. In an embodiment, the method comprises determining the presence in Enterobacter spp. of at least one difference in at least one nucleotide sequence identified in rows 698 to 700, 1075 to 1096, 1099 to 1103, 1105, 1106, 1108 to 1189, 1202 to 1212, 1227, 1229, 1230, 1234, 1240, and 1242 to 1245 of Table I compared with a reference sequence.

In an embodiment, the method comprises determining the absence in Enterobacter spp. of at least one nucleotide sequence identified in rows 718, 989, 990, 1034, 1044 to 1046, 1051, 1196, 1215, 1221, 1223 to 1225, 1233, 1235 to 1237, and 1247 of Table I or a variant of the nucleotide sequence. In an embodiment, the method comprises determining the absence in Enterobacter spp. of at least one difference in at least one nucleotide sequence identified in rows 701, 1098, 1104, 1107, 1228, and 1241 of Table I compared with a reference sequence.

Where the microorganism is Staphylococcus spp., preferably Staphylococcus aureus, the AMRs that when present or absent are indicative of resistance to at least one antimicrobial drug are preferably selected from those identified in rows 3376 to 3745 of Table I. The AMRs can further comprise those identified in rows 3824 to 3839 of Table I. A preferred difference in the nucleotide sequence identified in rows 3365 to 3745 of Table I is set forth in each of the same respective rows of Table I. The preferred at least one antimicrobial drug to which resistance is indicated by the presence of the AMR is disclosed for the same respective marker id of the AMR in Table II.

Those AMRs when present in Staphylococcus spp. which are indicative of resistance to at least one antimicrobial drug are preferably selected from those identified in rows 3376 to 3385, 3387 to 3393, 3395 to 3398, 3401, 3402, 3404, 3405, 3407, 3410 to 3412, 3414, 3416, 3418 to 3422, 3425 to 3429, 3431 to 3435, 3437 to 3441, 3443 to 3448, 3450, 3451, 3454 to 3461, 3463 to 3469, 3471 to 3475, 3477 to 3494, 3496 to 3506, 3508 to 3510, 3513 to 3528, 3533 to 3577, 3582 to 3593, 3595 to 3601, 3603 to 3652, 3654 to 3669, 3672 to 3675, 3678, 3679, 3681 to 3689, 3691, 3692, 3695 to 3701, 3703, 3704, 3706 to 3708, 3710 to 3722, 3724, 3728 to 3730, 3734 to 3736, 3738, 3741, and 3743 to 3745 of Table I.

Those AMRs when absent in Staphylococcus spp. which are indicative of resistance to at least one antimicrobial drug are preferably selected from those identified in rows 3386, 3394, 3399, 3400, 3423, 3424, 3430, 3436, 3442, 3449, 3452, 3453, 3462, 3470, 3476, 3495, 3507, 3511, 3512, 3529 to 3532, 3578 to 3581, 3594, 3602, 3653, 3670, 3671, 3676, 3677, 3680, 3690, 3693, 3694, 3702, 3705, 3709, 3723, 3725 to 3727, 3731 to 3733, 3737, 3739, 3740, and 3742 of Table I.

Those AMRs which involve a nucleotide sequence that when present in Staphylococcus spp. are indicative of resistance to at least one antimicrobial drug are preferably selected from those identified in rows 3376 to 3385, 3387 to 3393, 3395 to 3398, 3401, 3402, 3404, 3405, 3407, 3410 to 3412, 3414, 3416, 3418 to 3422, 3425 to 3429, 3431 to 3435, 3437 to 3441, 3443 to 3448, 3450, 3451, 3454 to 3461, 3463 to 3469, 3471 to 3475, 3477 to 3494, 3496 to 3506, 3508 to 3510, 3513 to 3528, 3533 to 3577, 3582 to 3593, 3595 to 3601, 3603 to 3652, 3654 to 3664 of Table I. Further, the AMRs which involve at least one difference in at least one nucleotide sequence compared to a reference sequence that when present are indicative of resistance to at least one antimicrobial drug are preferably selected from those identified in rows 3665 to 3669, 3672 to 3675, 3678, 3679, 3681 to 3689, 3691, 3692, 3695 to 3701, 3703, 3704, 3706 to 3708, 3710 to 3722, 3724, 3728 to 3730, 3734 to 3736, 3738, 3741, and 3743 to 3745 in Table I.

Those AMRs which involve a nucleotide sequence that when absent in Staphylococcus spp. are indicative of resistance to at least one antimicrobial drug are preferably selected from those identified in rows 3386, 3394, 3399, 3400, 3423, 3424, 3430, 3436, 3442, 3449, 3452, 3453, 3462, 3470, 3476, 3495, 3507, 3511, 3512, 3529 to 3532, 3578 to 3581, 3594, 3602, 3653 of Table I. Further, the AMRs which involve at least one difference in at least one nucleotide sequence compared to a reference sequence that when absent are indicative of resistance to at least one antimicrobial drug are preferably selected from those identified in rows 3670, 3671, 3676, 3677, 3680, 3690, 3693, 3694, 3702, 3705, 3709, 3723, 3725 to 3727, 3731 to 3733, 3737, 3739, 3740, and 3742 in Table I.

It is intended that the foregoing disclosure regarding the AMRs for Staphylococcus spp. be equally applicable to all of the methods disclosed herein, in particular those methods which comprise steps of determining the presence or absence of nucleotide sequences and/or differences in nucleotide sequences. In an embodiment, those AMRs for Staphylococcus spp. having p values not greater than 0.1, 0.01, 0.001, 0.0001, 10⁻⁵, 10⁻¹⁰, or 10⁻²⁰ as set forth in Table II are the AMRs used in the methods disclosed herein to indicate antimicrobial drug resistance. In an embodiment, those AMRs for Staphylococcus spp. having ppv values greater than 0.7, 0.75, 0.8, 0.85, 0.9, 0.95, or 0.98 as set forth in Table II are the AMRs used in the methods disclosed herein to indicate antimicrobial drug resistance. In an embodiment, those AMRs for Staphylococcus spp. having p values not greater than 0.1, 0.01, 0.001, 0.0001, 10⁻⁵, 10⁻¹⁰, or 10⁻²⁰ as set forth in Table II and having ppv values greater than 0.7, 0.75, 0.8, 0.85, 0.9, 0.95, or 0.98 as set forth in Table II are the AMRs used in the methods disclosed herein to indicate antimicrobial drug resistance.

In accordance with the foregoing, in an exemplary embodiment, where the microorganism is Staphylococcus, the methods disclosed herein comprise (i) determining the presence and/or absence in Staphylococcus spp. of at least one nucleotide sequence identified in rows 3376 to 3664 of Table I or a variant of the nucleotide sequence, and/or (ii) determining the presence and/or absence in Staphylococcus spp., of at least one difference in at least one nucleotide sequence identified in rows 3665 to 3745 of Table I compared with a reference sequence.

In an embodiment, the method comprises determining the presence in Staphylococcus spp. of at least one nucleotide sequence identified in rows 3376 to 3385, 3387 to 3393, 3395 to 3398, 3401, 3402, 3404, 3405, 3407, 3410 to 3412, 3414, 3416, 3418 to 3422, 3425 to 3429, 3431 to 3435, 3437 to 3441, 3443 to 3448, 3450, 3451, 3454 to 3461, 3463 to 3469, 3471 to 3475, 3477 to 3494, 3496 to 3506, 3508 to 3510, 3513 to 3528, 3533 to 3577, 3582 to 3593, 3595 to 3601, 3603 to 3652, 3654 to 3664 or a variant of the nucleotide sequence. In an embodiment, the method comprises determining the presence in Staphylococcus spp. of at least one difference in at least one nucleotide sequence identified in rows 3665 to 3669, 3672 to 3675, 3678, 3679, 3681 to 3689, 3691, 3692, 3695 to 3701, 3703, 3704, 3706 to 3708, 3710 to 3722, 3724, 3728 to 3730, 3734 to 3736, 3738, 3741, and 3743 to 3745 of Table I compared with a reference sequence.

In an embodiment, the method comprises determining the absence in Staphylococcus spp. of at least one nucleotide sequence identified in rows 3386, 3394, 3399, 3400, 3423, 3424, 3430, 3436, 3442, 3449, 3452, 3453, 3462, 3470, 3476, 3495, 3507, 3511, 3512, 3529 to 3532, 3578 to 3581, 3594, 3602, 3653 of Table I or a variant of the nucleotide sequence. In an embodiment, the method comprises determining the absence in Staphylococcus spp. of at least one difference in at least one nucleotide sequence identified in rows 3670, 3671, 3676, 3677, 3680, 3690, 3693, 3694, 3702, 3705, 3709, 3723, 3725 to 3727, 3731 to 3733, 3737, 3739, 3740, and 3742 of Table I compared with a reference sequence.

Where the microorganism is Raoultella spp., preferably Raoultella ornithinolytica or Raoultella planticola, the AMRs that when present or absent are indicative of resistance to at least one antimicrobial drug are preferably selected from those identified in rows 2879 to 2884 of Table I. A preferred difference in the nucleotide sequence identified in rows 2882 and 2884 of Table 1 is set forth in each of the same respective rows of Table I. The preferred at least one antimicrobial drug to which resistance is indicated by the presence or absence of the AMR is disclosed for the same respective marker ID of the AMR in Table II.

Those AMRs when present in Raoultella spp. which are indicative of resistance to at least one antimicrobial drug are preferably selected from those identified in rows 2879 to 2882 of Table I.

Those AMRs when absent in Raoultella spp. which are indicative of resistance to at least one antimicrobial drug are preferably selected from those identified in rows 2883 and 2884 of Table I.

Those AMRs which involve a nucleotide sequence that when present in Raoultella spp. are indicative of resistance to at least one antimicrobial drug are preferably selected from those identified in rows 2879 to 2881 of Table I. Further, the AMRs which involve at least one difference in at least one nucleotide sequence compared to a reference sequence that when present are indicative of resistance to at least one antimicrobial drug are preferably selected from those identified in rows 2882 in Table I.

Those AMRs which involve a nucleotide sequence that when absent in Raoultella spp. are indicative of resistance to at least one antimicrobial drug are preferably selected from those identified in row 2883 of Table I. Further, the AMRs which involve at least one difference in at least one nucleotide sequence compared to a reference sequence that when absent are indicative of resistance to at least one antimicrobial drug are preferably selected from those identified in row 2884 in Table I.

Those AMRs when present in Raoultella ornithinolytica which are indicative of resistance to at least one antimicrobial drug are preferably selected from those identified in rows 2879 to 2882 of Table I.

Those AMRs when absent in Raoultella planticola which are indicative of resistance to at least one antimicrobial drug are preferably selected from those identified in rows 2883 and 2884 of Table I.

In embodiments where the Raoultella microorganism is a particular species of Raoultella, the methods preferably comprise determining the presence and/or absence of the AMRs determined to be indicative of antimicrobial drug resistance in that species set forth in Table I.

It is intended that the foregoing disclosure regarding the AMRs for Raoultella spp. be equally applicable to all of the methods disclosed herein, in particular those methods which comprise steps of determining the presence or absence of nucleotide sequences and/or differences in nucleotide sequences. In an embodiment, those AMRs for Raoultella spp. having p values not greater than 0.1, 0.01, 0.001, 0.0001, 10⁻⁵, 10⁻¹⁰, or 10⁻²⁰ as set forth in Table II are the AMRs used in the methods disclosed herein to indicate antimicrobial drug resistance. In an embodiment, those AMRs for Raoultella spp. having ppv values greater than 0.7, 0.75, 0.8, 0.85, 0.9, 0.95, or 0.98 as set forth in Table II are the AMRs used in the methods disclosed herein to indicate antimicrobial drug resistance. In an embodiment, those AMRs for Raoultella spp. having p values not greater than 0.1, 0.01, 0.001, 0.0001, 10⁻⁵, 10⁻¹⁰, or 10⁻²⁰ as set forth in Table II and having ppv values greater than 0.7, 0.75, 0.8, 0.85, 0.9, 0.95, or 0.98 as set forth in Table II are the AMRs used in the methods disclosed herein to indicate antimicrobial drug resistance.

In accordance with the foregoing, in an exemplary embodiment, where the microorganism is Raoultella, the methods disclosed herein comprise (i) determining the presence and/or absence in Raoultella spp. of at least one nucleotide sequence identified in rows 2879 to 2881, and 2883 of Table I or a variant of the nucleotide sequence, and/or (ii) determining the presence and/or absence in Raoultella spp., of at least one difference in at least one nucleotide sequence identified in rows 2882 and 2884 of Table I compared with a reference sequence.

In an embodiment, the method comprises determining the presence in Raoultella spp. of at least one nucleotide sequence identified in rows 2879 to 2881 of Table I or a variant of the nucleotide sequence. In an embodiment, the method comprises determining the presence in Raoultella spp. of at least one difference in at least one nucleotide sequence identified in rows 2882 of Table I compared with a reference sequence.

In an embodiment, the method comprises determining the absence in Raoultella spp. of at least one nucleotide sequence identified in row 2883 of Table I or a variant of the nucleotide sequence. In an embodiment, the method comprises determining the absence in Raoultella spp. of at least one difference in at least one nucleotide sequence identified in row 2884 of Table I compared with a reference sequence.

Preferred AMRs whose presence or absence is determined are those having p values of less than 10′, more preferably less than 10⁻³, 10⁻⁴, 10⁻⁵, 10⁻¹⁰, 10⁴⁵, 10⁻²⁰, 10′⁵, 10⁻³°, 10⁻⁴°. The p value for each AMR is set forth in Table II. In an embodiment, the antimicrobial drug resistance status is indicated by the presence and/or absence of AMRs having a p value of less than 10⁻², 10⁻³, 10⁻⁴, 10⁻⁵, 10⁻¹⁰, 10⁻¹⁵, 10⁻²⁰, 10⁻²⁵, 10⁻³⁰, 10⁻⁴⁰.

Preferred AMRs whose presence or absence is determined are those having positive predictive value (ppv) of greater than 0.6, 0.65, 0.7, 0.75, 0.8, 0.85, 0.9, 0.95, 0.96, 0.97, or 0.98. The ppv for each AMR is set forth in Table II. In an embodiment, the antimicrobial drug resistance status is indicated by the presence and/or absence of AMRs having a ppv of at least 0.6, 0.65, 0.7, 0.75, 0.8, 0.85, 0.9, 0.95, 0.96, 0.97, or 0.98. In certain embodiments, preferred AMRs are those meeting threshold values of one or of combinations of particular values for p value, ppv, npv, specificity, sensitivity, specificity, and/or accuracy.

The determination of the presence or absence of nucleotide sequences and/or the differences in nucleotide sequences in the microorganism can be carried out using nucleic acid sequencing techniques, preferably, high throughput sequencing, e.g., “next generation” or third generation sequencing, such as by the Illumina/Solexa or the Oxford Nanopore methodology.

In some embodiments, the entire genome of the microorganism and any extra-genomic nucleic acids present in the microorganism are sequenced. In some embodiments, only the coding sequences of the genome/extra-genomic nucleic acids are sequenced. In some embodiments, only the non-coding sequences of the genomic/extra-genomic nucleic acids are sequenced. The extra-genomic nucleic acids can be naturally occurring in the microorganism or are not naturally occurring, for example, an artificial plasmid transformed into the microorganism.

In an embodiment, the difference in the nucleotide sequence is a point mutation, optionally resulting in a change in the amino acid sequence of the encoded amino acid sequence. In an embodiment, the point mutation does not result in the creation of a stop codon. In an embodiment, the point mutation results in the creation of a stop codon. In embodiments where the presence or absence of a difference in a nucleotide sequence is indicative of antimicrobial drug resistance, the nucleotide sequence is a nucleic acid of the microorganism, such as a gene, that preferably encodes a protein/peptide, and the difference can be a base substitution or an insertion or deletion of one or more bases, e.g., allowing for an in-frame deletion or addition in the encoded protein/peptide. The difference can also be the result of a gene fusion.

In an embodiment of the invention, one or more of the AMRs identified in Table I, whose nucleotide and amino acid sequences are set forth in the Sequence Listing, can be used as antimicrobial drug resistance markers.

The term “AMR” refers herein to a genetic marker which is a nucleotide sequence or difference in a nucleotide sequence compared to a corresponding reference sequence associated with antimicrobial drug resistance, particularly antibiotic resistance. In certain embodiments, the presence or absence of such AMR in the genomic or extra-genomic DNA of a microorganism, particularly bacterium, indicates antimicrobial drug resistance. The AMR may be a gene or a naturally occurring variant thereof. In certain embodiments, the AMR may be a coding region of a gene or a naturally occurring variant thereof. In certain embodiments, the AMR may be a non-coding region of a gene or a naturally occurring variant thereof. In certain embodiments, the AMR may be an intergenic region or a naturally occurring variant thereof, and thus not a gene. In certain embodiments, AMRs associated with antimicrobial drug resistance are specific mutations or differences in a nucleotide sequence compared to a corresponding wild type/reference sequence.

The terms “subject” or “patient” are used interchangeably and relate to vertebrates, preferably mammals. For example, mammals in the context of the present invention are humans, non-human primates, domesticated animals such as dogs, cats, sheep, cattle, goats, pigs, horses etc., laboratory animals such as mice, rats, rabbits, fish, guinea pigs, etc. as well as animals in captivity such as animals of zoos. The term “animal” also includes humans. Preferably, the terms “subject” or “patient” refer to male and female mammals, in particular male and female humans. The subject can be of any age, including neonates (e.g., from birth to about 6 months), infants (e.g., from about 6 months to about 2 years), children (e.g., from about 2 years to about 10 years), adolescents (e.g., from about 10 years to about 21 years), and adults (e.g., about 21 years and older).

As used herein, “sample” includes any biological sample obtained from a subject, e.g., from the body of the subject. Examples of such biological samples include whole blood, blood fractions such as plasma, serum, smears or swabs of a tissue, sputum, bronchial aspirate, urine, semen, stool, bile, gastrointestinal secretions, reproductive system secretions, lymph fluid, liquor, bone marrow, organ aspirates and tissue biopsies, including punch biopsies. Optionally, the biological sample can be obtained from a mucous membrane of the patient. The term “sample” can also include processed biological samples such as fractions or isolates, e.g., nucleic acids or isolated cells. Preferably, the biological sample contains nucleic acids, e.g., DNA or mRNA, such that the sequence of the nucleic acids can be determined. In an embodiment, the sample can be one that is obtained from a tissue showing signs of a disease state, e.g., showing signs of infection. In a preferred embodiment, the sample is blood or blood plasma obtained from the subject. The sample is analyzed according to the methods of the invention and during the method or thereafter is not normally returned to the body. In most embodiments, the presence of the subject's body is not necessary in order to carry out the methods of the invention.

Tissues of the patient from which the sample can be obtained include, but are not limited to, throat, mouth, nasal, stomach, intestinal, skin, liver, pancreatic, lung, neuronal cervical, vaginal, urethral, rectal, penial, and muscle. Any suitable method for obtaining the sample from the patient and/or from an appropriate tissue can be used in connection with the present invention.

In one embodiment, the sample is blood plasma, preferably obtained directly from the subject. The blood plasma is preferably cell-free, preferably mainly/mostly cell-free, e.g., fewer than 10,000, 1,000, 100, or 10 cells per mL. The biological sample, e.g., blood plasma, may contain free circulating nucleic acids, comprising nucleic acids of the subject and nucleic acids not of the subject, e.g., those of a microorganism. In one embodiment the biological sample can be diluted or concentrated. In another embodiment the sample is processed prior to sequencing, preferably the sample is purified to remove cellular components, such as lipids and proteins, prior to sequencing.

The term “in vivo” relates to the situation in a subject.

The term “genome” relates to the total amount of genetic information in the chromosomes of a cell.

The term “exome” refers to part of the genome formed by exons, which are coding portions of expressed genes. The exome provides the genetic blueprint used in the synthesis of proteins and other functional gene products. It is the most functionally relevant part of the genome and, therefore, it is most likely to contribute to the phenotype of an organism.

The terms “extra-genomic nucleic acids” or “extra-genomic DNA” as used herein may refer to naturally occurring extra-genomic nucleic acids or extra-genomic DNA in a microorganism, preferably in a bacterium, or to not naturally occurring extra-genomic nucleic acids or extra-genomic DNA, as for example, an artificial plasmid transformed into the microorganism, or a plasmid acquired from another microorganism, preferably another bacterium, e.g., due to bacterial conjugation.

The term “bacterial conjugation” as used herein denotes the transfer of genetic material between bacterial cells by direct cell-to-cell contact or by a bridge-like connection between two cells. It is a mechanism of horizontal gene transfer as are transformation and transduction; although these two other mechanisms do not involve cell-to-cell contact.

The term “transcriptome” relates to the set of all RNA molecules, including mRNA, rRNA, tRNA, and other non-coding RNA produced in one cell or a population of cells. In context of the present invention the transcriptome means the set of all RNA molecules produced in one cell, a population of cells, or all cells of a given individual at a certain time point.

The term “genetic material” includes isolated nucleic acid, either DNA or RNA, a section of a double helix, a section of a chromosome, or an organism's or cell's entire genome, in particular its exome or transcriptome.

According to the invention, “nucleic acid” is preferably deoxyribonucleic acid (DNA) or ribonucleic acid (RNA). Nucleic acids include genomic DNA, cDNA, mRNA, recombinantly produced and chemically synthesized molecules. A nucleic acid may be present as a single-stranded or double-stranded and linear or covalently circularly closed molecule, as well as mixtures thereof. A nucleic acid can be isolated. Preferably, the nucleic acid is a free circulating DNA and/or RNA molecule. In one embodiment, the term “nucleic acid” is also understood to mean “nucleic acid sequence”. Further, prior to sequencing, the nucleic acids can be processed, for example, enriched or amplified. In cases where the nucleic acid obtained from the sample is RNA, the RNA can be reverse transcribed into DNA for sequencing or the RNA itself can be sequenced.

The term “mutation” refers to a change of or a difference in the nucleic acid sequence (nucleotide substitution, addition or deletion) compared to a reference. According to the invention, the term “mutation” includes point mutations, indels, fusions, chromothripsis and RNA edits.

According to the invention, the term “indel” describes a special mutation class, defined as a mutation resulting in a co-localized insertion and deletion and a net gain or loss in nucleotides. In coding regions of the genome, unless the length of an indel is a multiple of 3, they produce a frameshift mutation. Indels can be contrasted with a point mutation; where an indel inserts and deletes nucleotides from a sequence, a point mutation is a form of substitution that replaces one of the nucleotides.

According to the invention, the term “chromothripsis” refers to a genetic phenomenon by which specific regions of the genome are shattered and then stitched together via a single devastating event.

Fusions can generate hybrid genes formed from two previously separate genes. It can occur as the result of a translocation, interstitial deletion, or chromosomal inversion. Often, fusion genes are oncogenes. Oncogenic fusion genes may lead to a gene product with a new or different function from the two fusion partners.

In context of the present invention, the term “sequencing” means to determine the sequence of at least one nucleic acid, and it includes any method that is used to determine the order of the bases in a strand of at least one nucleic acid. A preferred method of sequencing is high-throughput sequencing, such as next-generation sequencing or third generation sequencing.

For clarification purposes: the terms “Next Generation Sequencing” or “NGS” in the context of the present invention mean all high throughput sequencing technologies which, in contrast to the “conventional” sequencing methodology known as Sanger chemistry, read nucleic acid templates randomly in parallel along the entire genome by breaking the entire genome into small pieces. Such NGS technologies (also known as massively parallel sequencing technologies) are able to deliver nucleic acid sequence information of a whole genome, exome, transcriptome (all transcribed sequences of a genome) or methylome (all methylated sequences of a genome) in very short time periods, e.g., within 1-2 weeks, preferably within 1-7 days or most preferably within less than 24 hours and allow, in principle, single cell sequencing approaches. Multiple NGS platforms which are commercially available or which are mentioned in the literature can be used in the context of the present invention, e.g., those described in detail in Zhang et al., 2011, The impact of next-generation sequencing on genomics. J. Genet Genomics 38:95-109; or in Voelkerding et al., 2009, Next generation sequencing: From basic research to diagnostics, Clinical chemistry 55:641-658. Non-limiting examples of such NGS technologies/platforms are
1) The sequencing-by-synthesis technology known as pyrosequencing implemented, e.g., in the GS-FLX 454 Genome Sequencer™ of Roche-associated company 454 Life Sciences (Branford, Conn.), first described in Ronaghi et al., 1998, A sequencing method based on real-time pyrophosphate, Science 281:363-365. This technology uses an emulsion PCR in which single-stranded DNA binding beads are encapsulated by vigorous vortexing into aqueous micelles containing PCR reactants surrounded by oil for emulsion PCR amplification. During the pyrosequencing process, light emitted from phosphate molecules during nucleotide incorporation is recorded as the polymerase synthesizes the DNA strand.
2) The sequencing-by-synthesis approaches developed by Solexa (now part of Illumina Inc., San Diego, Calif.) which is based on reversible dye-terminators and implemented, e.g., in the Illumina/Solexa Genome Analyzer™ and in the Illumina HiSeq 2000 Genome Analyzer™. In this technology, all four nucleotides are added simultaneously into oligo-primed cluster fragments in flow-cell channels along with DNA polymerase. Bridge amplification extends cluster strands with all four fluorescently labeled nucleotides for sequencing.
3) Sequencing-by-ligation approaches, e.g., implemented in the SOLid™ platform of Applied Biosystems (now Life Technologies Corporation, Carlsbad, Calif.). In this technology, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting bead, each containing only copies of the same DNA molecule, are deposited on a glass slide. As a second example, the Polonator™ G.007 platform of Dover Systems (Salem, N.H.) also employs a sequencing-by-ligation approach by using a randomly arrayed, bead-based, emulsion PCR to amplify DNA fragments for parallel sequencing.
4) Single-molecule sequencing technologies such as, e.g., implemented in the PacBio RS system of Pacific Biosciences (Menlo Park, Calif.) or in the HeliScope™ platform of Helicos Biosciences (Cambridge, Mass.). The distinct characteristic of this technology is its ability to sequence single DNA or RNA molecules without amplification, defined as Single-Molecule Real Time (SMRT) DNA sequencing. For example, HeliScope uses a highly sensitive fluorescence detection system to directly detect each nucleotide as it is synthesized. A similar approach based on fluorescence resonance energy transfer (FRET) has been developed from Visigen Biotechnology (Houston, Tex.). Other fluorescence-based single-molecule techniques are from U.S. Genomics (GeneEngine™) and Genovoxx (AnyGene™)
5) Nano-technologies for single-molecule sequencing in which various nanostructures are used which are, e.g., arranged on a chip to monitor the movement of a polymerase molecule on a single strand during replication. Non-limiting examples for approaches based on nano-technologies are the GridON™ platform of Oxford Nanopore Technologies (Oxford, UK), the hybridization-assisted nano-pore sequencing (HANS™) platforms developed by Nabsys (Providence, R.I.), and the proprietary ligase-based DNA sequencing platform with DNA nanoball (DNB) technology called combinatorial probe-anchor ligation (cPAL™)
6) Electron microscopy based technologies for single-molecule sequencing, e.g., those developed by LightSpeed Genomics (Sunnyvale, Calif.) and Halcyon Molecular (Redwood City, Calif.)
7) Ion semiconductor sequencing which is based on the detection of hydrogen ions that are released during the polymerization of DNA. For example, Ion Torrent Systems (San Francisco, Calif.) uses a high-density array of micro-machined wells to perform this biochemical process in a massively parallel way. Each well holds a different DNA template. Beneath the wells is an ion-sensitive layer and beneath that a proprietary Ion sensor.

Other sequencing methods useful in the context of the invention include tunneling currents sequencing (Xu et al., 2007, The electronic properties of DNA bases, Small 3:1539-4543, Di Ventra, 2013, Fast DNA sequencing by electrical means inches closer, Nanotechnology 24:342501). Particularly preferable next-generation sequencing (NGS) methodologies include Illumina, IONTorrent and NanoPore sequencing.

Preferably, DNA and RNA preparations serve as starting material for NGS. Such nucleic acids can be easily obtained from biological samples, e.g., from blood or fresh, flash-frozen or formalin-fixed tissue samples or from freshly isolated cells. Although nucleic acids extracted can be highly fragmented, they are nonetheless suitable for NGS applications.

Several targeted NGS methods for exome sequencing are described in the literature (for review see, e.g., Teer and Mullikin, 2010, Human Mol Genet 19:R145-51), all of which can be used in conjunction with the present invention. Many of these methods (described, e.g., as genome capture, genome partitioning, genome enrichment, etc.) use hybridization techniques and include array-based (e.g., Hodges et al., 2007, Nat Genet 39:1522-1527) and liquid-based (e.g., Choi et al., 2009, Proc Natl Acad Sci USA 106:19096-19101) hybridization approaches. Commercial kits for DNA sample preparation and subsequent exome capture are also available: for example, Illumina Inc. (San Diego, Calif.) offers the TruSeq™ DNA Sample Preparation Kit and the Exome Enrichment Kit TruSeq™ Exome Enrichment Kit.

In certain embodiments of the invention, in order to reduce the number of false positive findings, it is preferred to determine/compare the sequences in replicates. Thus, it is preferred that nucleic acid sequences in a biological sample be sequenced twice, three times or more. In one embodiment, the sequencing of nucleic acid sequences obtained from a sample is determined twice, three times or more. It may also be possible to determine the sequence more than once by determining at least once the sequence in genomic DNA and determining at least once the sequence in RNA of said sample. For example, by determining the variations between replicates of a sample, the expected rate of false positive (FDR) mutations as a statistical quantity can be estimated. Technical repeats of a sample should generate identical results and any detected mutation in this “same vs. same comparison” is a false positive. Furthermore, various quality related metrics (e.g., coverage or SNP quality) may be combined into a single quality score using a machine learning approach.

In context of the present invention, the term “database” relates to an organized collection of data, preferably as an electronic filing system. In an embodiment, a sequence database is a type of database that is composed of a collection of computerized (“digital”) nucleic acid sequences, protein sequences, or other polymer sequences stored on a computer. Preferably, the database is a collection of nucleic acid sequences, i.e., the genetic information from a number of species. The genetic information can be derived from the genome and/or the exome and/or the transcriptome of a species. Exemplary nucleic acid databases useful in the present invention include, but are not limited to, International Nucleotide Sequence Database (INSD), DNA Data Bank of Japan (National Institute of Genetics), EMBL (European Bioinformatics Institute), GenBank (National Center for Biotechnology Information), Bioinformatic Harvester, Gene Disease Database, SNPedia, CAMERA Resource for microbial genomics and metagenomics, EcoCyc (a database that describes the genome and the biochemical machinery of the model organism E. coli K-12), Ensembl (provides automatic annotation databases for human, mouse, other vertebrate and eukaryote genomes) Ensembl Genomes (provides genome-scale data for bacteria, protists, fungi, plants and invertebrate metazoa, through a unified set of interactive and programmatic interfaces (using the Ensembl software platform)), Exome Aggregation Consortium (ExAC) (exome sequencing data from a wide variety of large-scale sequencing projects (Broad Institute)), PATRIC (PathoSystems Resource Integration Center), MGI Mouse Genome (Jackson Laboratory), JGI Genomes of the DOE-Joint Genome Institute (provides databases of many eukaryote and microbial genomes), National Microbial Pathogen Data Resource (a manually curated database of annotated genome data for the pathogens Campylobacter, Chlamydia, Chlamydophila, Haemophilus, Listeria, Mycoplasma, Neisseria, Staphylococcus, Streptococcus, Treponema, Ureaplasma and Vibrio), RegulonDB (a model of the complex regulation of transcription initiation or regulatory network of the cell E. coli K-12), Saccharomyces Genome Database (genome of the yeast model organism), Viral Bioinformatics Resource Center (curated database containing annotated genome data for eleven virus families), The SEED platform (includes all complete microbial genomes, and most partial genomes, the platform is used to annotate microbial genomes using subsystems), WormBase ParaSite (parasitic species), UCSC Malaria Genome Browser (genome of malaria causing species (Plasmodium falciparum and others)), Rat Genome Database (genomic and phenotype data for Rattus norvegicus), INTEGRALL (database dedicated to integrons, bacterial genetic elements involved in the antibiotic resistance), VectorBase (NIAID Bioinformatics Resource Center for Invertebrate Vectors of Human Pathogens), EzGenome, comprehensive information about manually curated genome projects of prokaryotes (archaea and bacteria), GeneDB (Apicomplexan Protozoa, Kinetoplastid Protozoa, Parasitic Helminths, Parasite Vectors as well as several bacteria and viruses), EuPathDB (eukaryotic pathogen database resources includes amoeba, fungi, plasmodium, trypanosomatids etc.); The 1000 Genomes Project (providing the genomes of more than a thousand anonymous participants from a number of different ethnic groups), Personal Genome Project (providing human genomes).

Exemplary microorganisms encompassed within the methods of the invention include bacteria, fungi and parasites, preferably bacteria. Exemplary bacteria include, but are not limited to, those listed above and the following: Acinetobacter spp., such as Acinetobacter baumannii, Escherichia spp., such as Escherichia coli, Klebsiella spp., such as Klebsiella aerogenes, Klebsiella oxytoca, Klebsiella pneumoniae, Morganella spp., such as Morganella morganii, Proteus spp., such as Proteus mirabilis, Pseudomonas spp., such as Pseudomonas aeruginosa, Salmonella spp., such as Salmonella enterica, Serratia spp., such as Serratia marcescens, Stenotrophomonas spp., such as Stenotrophomonas maltophilia, Shigella spp., such as Shigella boydii, Shigella flexneri, Shigella sonnei, Enterobacter spp., such as Enterobacter cloacae, Staphylococcus spp., such as Staphylococcus aureus, Neisseria meningitis, Streptococcus pneumoniae, Streptococcus pyogenes, Moraxella catarrhalis, Bordetella pertussis, Clostridium tetani, Corynebacterium diphtheria, Haemophilus influenza, Streptococcus agalactiae, Chlamydia trachomatis, Chlamydia pneumoniae, Helicobacter pylori, Bacillus anthracis, Yersinia pestis, Staphylococcus epidermis, Clostridium perfringens, Clostridium botulinum, Legionella pneumophila, Coxiella burnetii, Brucella spp. such as B. abortus, B. canis, B. melitensis, B. neotomae, B. ovis, B. suis, B. pinnipediae, Francisella spp. such as F. novicida, F. philomiragia, F. tularensis, Neisseria gonorrhoeae, Treponema pallidum, Haemophilus ducreyi, Enterococcus faecalis, Enterococcus faecium, Staphylococcus saprophyticus, Yersinia enterocolitica, Mycobacterium tuberculosis, Rickettsia spp., Listeria monocytogenes, Vibrio cholera, Salmonella typhi, Borrelia burgdorferi, and Porphyromonas gingivalis.

In an embodiment, the term “percentage identity” within the context of nucleotide sequences is intended to denote a percentage of nucleotides which are identical between the two sequences to be compared, obtained after the best alignment, this percentage being purely statistical and the differences between the two sequences being distributed randomly and over their entire length. Sequence comparisons between two nucleic acid sequences are conventionally carried out by comparing these sequences after having aligned them optimally, said comparison being carried out by segment or by “window of comparison” in order to identify and compare local regions of sequence similarity. The optimal alignment of the sequences for comparison may be produced, besides manually, by means of the local homology algorithm of Smith and Waterman, 1981, Ads App. Math. 2, 482, by means of the local homology algorithm of Needleman and Wunsch, 1970, J. Mol. Biol. 48, 443, by means of the similarity search method of Pearson and Lipman, 1988, Proc. Natl Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

The percentage identity is calculated by determining the number of identical positions between the two nucleic sequences being compared, dividing this number by the number of positions compared and multiplying the result obtained by 100 so as to obtain the percentage identity between these two sequences.

The term “wild type” as used herein refers to the phenotype of the typical form of a species of a microorganism as it occurs in nature. However, most or all gene loci exist in a variety of allelic forms, which vary in frequency throughout the geographic range of a species, and it is held that in most instances a uniform wild type does not exist in nature. In general, however, the most prevalent allele, i.e., the one with the highest gene frequency, is the one deemed as wild type. Within the context of the present invention, the nucleotide and amino acid sequences depicted in the Sequence Listing, except for the “n” and “Xaa” positions refer to wild type sequences, of which naturally occurring variants exist, e.g., due to the said variety of allelic forms.

“Reference nucleotide sequences”, for instance, are not particularly limited to those set forth in the Sequence Listing and may be taken from one or more databases, such as those listed above. Depending on the microorganism to be tested, different reference nucleic acid sequences can be used for aligning; e.g., corresponding wild type gene sequences, mutated gene sequences associated with antimicrobial drug resistance, or intergenic nucleic acid sequences associated with antimicrobial drug resistance, preferably antibiotic resistance. Using such reference nucleotide sequences, nucleotide alterations present in the input nucleotide sequences may be obtained that mediate antimicrobial drug resistance in one or more microbial genus and species. Particularly, nucleotide alterations present in the input nucleotide sequences may be obtained that mediate antimicrobial drug resistance in one or more microorganisms of different genus and/or species.

In context of the present invention, the term “antibiotic resistance” means a loss of susceptibility of microorganism to the killing, or growth-inhibiting properties of an antibiotic agent. It also relates to resistance of a microorganism to an antimicrobial drug that was originally effective for treatment of infections caused by it. Resistant microorganisms, including bacteria, are able to withstand attack by antimicrobial drugs, such as antibacterial drugs, so that standard treatments become ineffective and infections persist.

In the context of the present invention, the terms “susceptible”, “susceptibility”, “sensitive” or “sensitivity” as used herein refer to the ability of a microorganism, preferably a bacterium, to be growth inhibited or killed by an antimicrobial drug.

In an embodiment, the present invention is directed also to a device for carrying out the method according to the present invention, wherein the presence or absence of nucleotide sequences and/or differences in nucleotide sequences to determine whether or not a microorganism is resistant to one or more antimicrobial drugs is computed by a central processing unit of the device. In an embodiment, the present invention is directed also to a device for carrying out the method according to the present invention, wherein (i) determining the presence or absence in the microorganism of at least one nucleotide sequence identified in Table I or a variant of the nucleotide sequence, and/or (ii) determining the presence or absence in the microorganism of at least one difference in at least one nucleotide sequence identified in Table I compared with a reference sequence, is computed by a central processing unit of the device. In an embodiment, the central processing unit is a field-programmable gate array (FPGA).

The methodology of the present invention is preferably not restricted to the determination of an antimicrobial drug resistance status of a single specific microorganism. In one embodiment, the present method determines the status for all microorganisms, e.g., present in a sample obtained from a patient, preferably all microorganisms relevant for an infection in the patient.

Thus, the present invention provides a useful method for determining the antimicrobial drug resistance status of a microorganism causing or suspected of causing an infection in a patient within short time, such that an appropriate therapy for the infection can be selected within short time, as well as to avoid non-effective therapies such as administering antimicrobials to which the microorganism is resistant.

The present invention is described in detail by the figures and examples below, which are used only for illustration purposes and are not meant to be limiting. Owing to the description and the examples, further embodiments which are likewise included in the invention are accessible to the skilled worker.

### FIGURES

FIG. 1 depicts a schematic diagram for the computational work-flow applied for the identification of AMRs.

### EXAMPLES

1. Determination of Antimicrobial Resistance Markers (AMRs)

Bacterial Isolates

The dataset of 11,087 isolates consisted of 1,001 isolates from the S. aureus strain collection of Saarland University Medical Center and a collection of 10,086 413 Gram-negative bacterial clinical isolates that form part of the microbiology strain collection of Siemens Healthcare Diagnostics (West Sacramento, Calif., 415 USA). DNA extraction using the Siemens VERSANT® sample preparation system and whole-genome next-generation sequencing were performed for all isolates (2×100 bp paired-end on Illumina Hiseq2000/2500 sequencers).

Susceptibility Testing and Resistance Profiles

For 993 isolates from the S. aureus strain collection detection of methicillin resistant and susceptible Staphylococcus aureus (MRSA/MSSA) isolates was performed. The specimen were plated on CHROMagar MRSA detection biplates (Mast, Germany). All MRSA positive culture isolates were further confirmed using a penicillin binding protein 2a latex agglutination test (Alere, Germany).

For 9,998 isolates from the Gram-negative isolate collection antimicrobial susceptibility testing (AST) was performed. Frozen reference AST panels were prepared following Clinical Laboratory Standards Institute (CLSI) recommendationsl. The antimicrobial agents included in the panels are set forth below in “Drug Information”. Prior to use with clinical isolates, AST panels were tested and considered acceptable for testing with clinical isolates when the QC results met QC ranges described by CLSI (CLSI Performance Standards for Antimicrobial Susceptibility Testing; Twenty-Fifth Informational Supplement. CLSI document M100-S25, Wayne, P. C. and L. S. I. 2015. M100-S25 Performance Standards for Antimicrobial. (2014)). Isolates were cultured on trypticase soy agar with 5% sheep blood (BBL, 436 Cockeysville, Md.) and incubated in ambient air at 35±1° C. for 18-24 h. Isolated colonies panels were inoculated according to CLSI recommendations (CLSI additional reference) and incubated in ambient air at 35±1° C. for 16-20 h. Panel results 439 were read visually, and minimal inhibitory concentrations (MIC) were determined.

Drug Information

The 25 used drugs were grouped into different drug classes based on their category in the EUCAST guidelines (EUCAST. European Committee on Antimicrobial Susceptibility Testing Antifungal Agents Breakpoint tables for interpretation of MICs. Eucast 1-5 (2017)): 5 drugs belong to cephalosporins (cefuroxime—2nd generation, cefotaxime, ceftazidime and ceftriaxone—3rd generation, cefepime—4th generation), 7 to penicillins, 3 to carbapenems, 3 to fluoroquinolones, 2 to aminoglycosides in addition to 1 tetracycline, 1 monobactam, 1 folate pathway inhibitor, 1 lincosamide and 1 macrolide.

Antimicrobial Marker Extraction

Antimicrobial markers were extracted per species and antimicrobial compound.

Data Preparation

Genotype matrix (X): A sample by feature presence/absence matrix of genes, single nucleotide variants, and small genetic variations was compiled. Features were encoded categorically into 0 (absence) and 1 (presence). Nearly constant features and sparse features with missing values are removed.

Phenotype vector (y): Associated sample-wise minimum inhibitory concentrations were transformed into categorical labels for antimicrobial resistance (R) and antimicrobial susceptibility (S) using EUCAST guidelines v. 7.1 2. Intermediate values (I) were converted to resistance value (R).

Training/Test sets: Prior to marker extraction, i.e., feature selection, the data set (X/y) was split into a training (Xtrain/ytrain) and test (Xtest/ytest) set for independent validation using stratified sampling on the phenotype (y) and information about population structure consisting of the year the sample was isolated, name of the submitting organization, geographical location, t-SNE projected genomic distance, and multi-compound resistance information.

Data Processing

Extracted markers, so called Self-Consistent Feature Sets, were derived from the consensus of multiple individual Genome-Wide-Analysis (GWAS) runs on bootstrapped training sets in order to increase the robustness of the extracted markers (FIG. 1).

Bootstrapping: Multiple training subsets (Xtrain′) were created by repeated sampling with replacement on the original training set (Xtrain) prior to GWAS and feature extraction.

GWAS: The training set (Xtrain) was subjected to GWAS for feature selection—once prior to bootstrapping and ten times (m) after bootstrapping. MatrixEQTL (Shabalin, 2012, Matrix eQTL: Ultra fast eQTL analysis via large matrix operations, Bioinformatics (doi:10.1093/bioinformatics/bts163)) was used for association testing between individual features and phenotypes using linear regression. Covariates as listed in section ‘Training/Test sets’ were added to the analysis with MatrixEQTL to account for the population structure and multi-compound resistance. Features were selected after each GWAS run based on a false discovery rate (FDR) of less than 0.05 (I) before the residual effect of other high-ranking features (II) was analyzed. These conditional analyses reduced the effect of already selected features over five iterations by addition of primary components as fixed effects from PLS-DA analyses on previously selected features and their associated phenotypes. Any additional features that came out of these conditional GWAS analyses with a FDR of less than 0.05 were added to the selected features. In addition, up to five top-ranking features according to PPV or NPV below the required FDR of 0.05 were also added to each feature set selection round. The filtered—feature selected—training subset (Xtrain′) contained the entirety of features from (I) and iterations in (II).

Feature Selection: The self-consistent feature set for a species-compound pair was defined as all significant features from the repeated GWAS feature selections runs that came up in at more than two runs in total.

Evaluation

The self-consistent feature set was evaluated using random forest models. Model parameters were optimized using exhaustive grid search and the model was trained with cross validation on a balanced training set of Xtrain′ before model performance was assessed using the test set (Xtest).

For every species-compound pair, the markers comprising the self-consistent feature set are reported in Tables I and II. To demonstrate the performance of said markers in predicting resistance against said compound in isolates of the said species, we report performance metrics such as sensitivity, specificity, accuracy, positive predictive value (ppv), and negative predictive value (npv), as set forth in Table II.

The reported performance metrics can be calculated for every feature from a confusion matrix, i.e., a table reflecting how many isolates are correctly and incorrectly labelled as resistant to a compound based on the presence (or absence) of a feature.

Table III presents a confusion matrix for binary classification, where tp are true positive, fp—false positive, fn—false negative, and to—true negative counts. Here, a positive case refers to resistance and a negative case refers to susceptibility.

TABLE III
A confusion matrix for binary classification.

Prediction

| | As Positive/ | As Negative/ |
|---|---|---|
| Actual | Resistant | Susceptible |
| Positive/Resistant | tp | fn |
| Negative/Susceptible | fp | tn |

According to scientific literature (e.g., Sokolova, et al., 2006, Beyond Accuracy, F-Score and ROC: A Family of Discriminant Measures for Performance Evaluation, pages 1015-1021 (Springer, Berlin, Heidelberg (doi:10.1007/11941439_1144)), accuracy, sensitivity, specificity, positive predictive value and negative predictive value are among the commonly accepted performance evaluation metrics that can be calculated from a confusion matrix (Table III).

$$accuracy = \frac{tp + tn}{tp + fp + fn + tn}$$

$$sensitivity = \frac{tp}{tp + fn}$$

$$specificity = \frac{tn}{fp + tn}$$

$$positive predicitve value = \frac{tp}{tp + fp}$$

$$negative predictive value = \frac{tn}{fn + tn}$$

Using the foregoing methodology, the AMRs that were identified in various microorganisms whose absence or presence in the microorganism is associated with/indicative of a microorganism being resistant to one or more antimicrobial drugs are listed in rows 0 to 3840 of Table I. Further, the subset of AMRs identified that indicate that the microorganism is resistant to more than one type/class of antimicrobial drug are listed in the rows of Table I in which a plus (+) is in the column “Marker associated with Cross-Resistance. The subset of AMRs identified that indicate antimicrobial resistance in more than one species of microorganism are listed in the rows of Table I in which a plus (+) is in the column “Associated with resistance in multiple pathogens”. Those AMRs identified that are associated with antimicrobial resistance to more than one type/class of antimicrobial drug in more than one species of microorganism are listed in the rows of Table I in which a plus (+) is in the column “cross-resistance in multiple pathogens.

2. Determining the Antimicrobial Drug Resistance Status of a Microorganism

A sample that has been obtained from a patient having or suspected of having an infection, which sample contains or is suspected of containing a microorganism causing or suspected of causing the infection in the patient, is processed using standard methods such that the genome and/or extra-genomic nucleic acids of the microorganism is sequenced using a high throughput method of sequencing.

The resulting sequence information is compared with a database containing the AMRs identified in Tables I and II along with their relevance to antimicrobial drug resistance, i.e., whether an individual AMR is present or absent in the microorganism indicates resistance to an antimicrobial drug, specifically the drug associated with the respective AMR. The comparison will result in a determination of the antimicrobial drug resistance status of the microorganism.

If the microorganism is not resistant to any antimicrobial drugs, the patient will be give any antimicrobial drug determined to be appropriate by the treating physician. If the microorganism is determined to be resistant to one or more antimicrobial drugs, an appropriate antimicrobial drug to which the microorganism is sensitive will be given to the patient.

Lengthy table referenced here
US20210002706A1-20210107-T00001
Please refer to the end of the specification for access instructions.

Lengthy table referenced here
US20210002706A1-20210107-T00002
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20210002706A1). An electronic copy of the table will also be available from the USPTO upon request and payment of the fee set forth in 37 CFR 1.19(b)(3).

1-34. (canceled)

35. A method for treating a patient suffering from an infection with an antimicrobial drug resistant microorganism, the method comprising the steps of:
(a) obtaining or providing a sample containing or suspected of containing at least one microorganism from the patient;
(b) performing or having performed a genotyping assay on the sample to determine (i) the presence or absence in the microorganism of at least one nucleotide sequence identified in Table I or a variant of the nucleotide sequence and/or (ii) the presence or absence in the microorganism of at least one difference in at least one nucleotide sequence identified in Table I compared with a reference sequence; wherein the presence or absence of the at least one nucleotide sequence or variant thereof and/or the difference indicates that the microorganism is resistant to one or more antimicrobial drugs;
(c) administering to the patient an antimicrobial drug that is different from the one or more antimicrobial drugs to which the microorganism is resistant.

36. The method of claim 35, wherein the microorganism is selected from the group consisting of Acinetobacter, Escherichia, Klebsiella, Morganella, Proteus, Pseudomonas, Salmonella, Serratia, Stenotrophomonas, Shigella, Enterobacter, Kluyvera, Burkholdia, Citrobacter, Pantoea, Raoultella, and Staphylococcus.

37. The method of claim 35, wherein the microorganism is (1) an Acinetobacter species, (2) an Escherichia species, (3) a Klebsiella species, (4) a Morganella species, (5) a Proteus species, (6) a Pseudomonas species, (7) a Salmonella species, (8) a Serratia species, (9) a Stenotrophomonas species, (10) a Shigella species, (11) an Enterobacter species, (12) a Staphylococcus species, (13) a Burkholderia species, (14) a Citrobacter species, (15) a Kluyvera species, (16) a Pantoea species, or (17) a Raoultella species; and
wherein step (b) comprises:
(b′ 1) performing or having performed a genotyping assay on the sample to determine (i) the presence or absence in the Acinetobacter species of at least one nucleotide sequence identified in rows 0 to 183, 282 to 289, and 291 to 325 of Table I or a variant of the nucleotide sequence, and/or (ii) the presence in the Acinetobacter species of at least one difference in at least one nucleotide sequence identified in rows 184 to 281, 290, and 326 to 330 of Table I compared with a reference sequence;
(b′2) performing or having performed a genotyping assay on the sample to determine (i) the presence or absence in the Escherichia species of at least one nucleotide sequence identified in rows 1248 to 1499 of Table I or a variant of the nucleotide sequence, and/or (ii) the presence in the Escherichia species of at least one difference in at least one nucleotide sequence identified in rows 1500 to 1586 of Table I compared with a reference sequence;
(b′3) performing or having performed a genotyping assay on the sample to determine (i) the presence or absence in the Klebsiella species of at least one nucleotide sequence identified in rows 1587 to 1696, 1743 to 1782, 1792 to 2133, 2208 to 2396, 2425 to 2427, and 2431 to 2446 of Table I or a variant of the nucleotide sequence, and/or (ii) the presence and/or absence in the Klebsiella species of at least one difference in at least one nucleotide sequence identified in rows 1697 to 1742, 1783 to 1791, 2134 to 2207, 2397 to 2424, 2428 to 2430, 2447, and 2448 of Table I compared with a reference sequence;
(b′4) performing or having performed a genotyping assay on the sample to determine (i) the presence or absence in the Morganella species of at least one nucleotide sequence identified in rows 2460 to 2569 of Table I or a variant of the nucleotide sequence, and/or (ii) the presence and/or absence in the Morganella species of at least one difference in at least one nucleotide sequence identified in rows 2570 to 2621 of Table I compared with a reference sequence;
(b′5) performing or having performed a genotyping assay on the sample to determine (i) the presence or absence in the Proteus species of at least one nucleotide sequence identified in rows 2623 to 2754, and 2789 to 2792 of Table I or a variant of the nucleotide sequence, and/or (ii) the presence and/or absence in the Proteus species of at least one difference in at least one nucleotide sequence identified in rows 2755 to 2788 of Table I compared with a reference sequence;
(b′6) performing or having performed a genotyping assay on the sample to determine (i) the presence or absence in the Pseudomonas species of at least one nucleotide sequence identified in rows 2793 to 2861, 2877, and 2878 of Table I or a variant of the nucleotide sequence, and/or (ii) the presence and/or absence in the Pseudomonas species of at least one difference in at least one nucleotide sequence identified in rows 2862 to 2876 of Table I compared with a reference sequence;
(b′7) performing or having performed a genotyping assay on the sample to determine (i) the presence or absence in the Salmonella species of at least one nucleotide sequence identified in rows 2885 to 2990 of Table I or a variant of the nucleotide sequence, and/or (ii) the presence in the Salmonella species of at least one difference in at least one nucleotide sequence identified in rows 2991 to 3008 of Table I compared with a reference sequence;
(b′8) performing or having performed a genotyping assay on the sample to determine (i) the presence or absence in the Serratia species of at least one nucleotide sequence identified in rows 3009 to 3013, 3023 to 3028, 3031 to 3131, and 3188 to 3198 of Table I or a variant of the nucleotide sequence, and/or (ii) the presence and/or absence in the Serratia species of at least one difference in at least one nucleotide sequence identified in rows 3014 to 3022, 3029, 3030, 3132 to 3187, 3199, and 3200 of Table I compared with a reference sequence;
(b′9) performing or having performed a genotyping assay on the sample to determine (i) the presence in the Stenotrophomonas species of at least one nucleotide sequence identified in rows 3746 to 3751 of Table I or a variant of the nucleotide sequence;
(b′10) performing or having performed a genotyping assay on the sample to determine (i) the presence or absence in the Shigella species of at least one nucleotide sequence identified in rows 3201 to 3234, 3237 to 3256, and 3263 to 3352 of Table I or a variant of the nucleotide sequence, and/or (ii) the presence in the Shigella species of at least one difference in at least one nucleotide sequence identified in rows 3235, 3236, 3257 to 3262, and 3353 to 3375 of Table I compared with a reference sequence;
(b′ 11) performing or having performed a genotyping assay on the sample to determine (i) the presence or absence in the Enterobacter species of at least one nucleotide sequence identified in rows 692 to 697, 702 to 1074, 1190 to 1201, 1213 to 1226, 1231 to 1233, 1235 to 1239, 1246, and 1247 of Table I or a variant of the nucleotide sequence, and/or (ii) the presence and/or absence in the Enterobacter species of at least one difference in at least one nucleotide sequence identified in rows 698 to 701, 1075 to 1189, 1202 to 1212, 1227 to 1230, 1234, and 1240 to 1245 of Table I compared with a reference sequence;
(b′12) performing or having performed a genotyping assay on the sample to determine (i) the presence or absence in the Staphylococcus species of at least one nucleotide sequence identified in rows 3376 to 3664 of Table I or a variant of the nucleotide sequence, and/or (ii) the presence in the Staphylococcus species of at least one difference in at least one nucleotide sequence identified in rows 3665 to 3745 of Table I compared with a reference sequence;
(b′13) performing or having performed a genotyping assay on the sample to determine (i) the presence or absence in the Burkholderia species of at least the nucleotide sequence identified in row 331 of Table I or a variant of the nucleotide sequence;
(b′14) performing or having performed a genotyping assay on the sample to determine (i) the presence or absence in the Citrobacter species of at least one nucleotide sequence identified in rows 332 to 342, 345 to 361, 365 to 419, 422 to 581, 634 to 650, 653 to 683, 690, and 691 of Table I or a variant of the nucleotide sequence, and/or (ii) the presence in the Citrobacter species of at least one difference in at least one nucleotide sequence identified in rows 343, 344, 362 to 364, 420, 421, 582 to 633, 651, 652, and 684 to 689 of Table I compared with a reference sequence;
(b′15) performing or having performed a genotyping assay on the sample to determine (i) the presence or absence in the Kluyvera species of at least one nucleotide sequence identified in rows 2449 to 2457 of Table I or a variant of the nucleotide sequence, and/or (ii) the presence in the Kluyvera species of at least one difference in at least one nucleotide sequence identified in rows 2458 to 2459 of Table I compared with a reference sequence;
(b′16) performing or having performed a genotyping assay on the sample to determine (i) the presence or absence in the Pantoea species of at least the nucleotide sequence identified in row 2622 of Table I or a variant of the nucleotide sequence; or
(b′17) performing or having performed a genotyping assay on the sample to determine (i) the presence or absence in the Raoultella species of at least one nucleotide sequence identified in rows 2879 to 2881, and 2883 of Table I or a variant of the nucleotide sequence, and/or (ii) the presence in the Raoultella species of at least one difference in at least one nucleotide sequence identified in rows 2882, and 2884 of Table I compared with a reference sequence.

38. The method of claim 37, wherein the genotyping assay comprises using a next generation sequencing or high throughput sequencing method.

39. The method of claim 37, wherein the difference in the nucleotide sequence is at position nc_pos of the nucleotide sequence identified in Table I.

40. The method of claim 39, wherein the difference at position nc_pos is the nc_alt residue for the nucleotide sequence identified in Table I.

41. The method of claim 37, wherein the difference in the nucleotide sequence is a point mutation resulting in a change in the encoded amino acid sequence.

42. The method of claim 37, wherein the one or more antimicrobial drugs to which the microorganism is resistant belong to a class of antimicrobial drugs selected from the group consisting of lactams; penicillins; quinolones and derivatives thereof; aminoglycosides; polyketides; benzene-derived compounds; sulfonamides;
tetracyclines; cephalosporins; lincosamides; macrolides; nitrofuranes; glycopeptides;
oxazolidinones; ansamycins; carbacephems; and folate synthesis inhibitors.

43. The method of claim 37, wherein the one or more antimicrobial drugs to which the microorganism is resistant is selected from the group consisting of amoxicillin/K clavulanate (AUG), ampicillin (AM), ampicillin/sulbactam (A/S), aztreonam (AZT), cefazolin (CFZ), cefepime (CPE), cefotaxime (CFT), ceftazidime (CAZ), ceftriaxone (CAX), cefuroxime (CRM), cephalotin (CF), ciprofloxacin (CP), ertapenem (ETP), gentamicin (GM), imipenem (IMP), levofloxacin (LVX), meropenem (MER), piperacillin/tazobactam (P/T), tetracycline (TE), tobramycin (TO), and trimethoprim/sulfamethoxazole (T/S).

44. A method for treating a patient infected with an antimicrobial drug resistant microorganism, the method comprising:
administering to the patient an antimicrobial drug capable of killing or inhibiting the growth of an antimicrobial drug resistant microorganism;
wherein prior to said administration, a sample containing or suspected of containing the antimicrobial drug resistant microorganism from the patient has been tested to determine (i) the presence or absence in the microorganism of at least one nucleotide sequence identified in Table I or a variant of the nucleotide sequence and/or (ii) the presence or absence in the microorganism of at least one difference in at least one nucleotide sequence identified in Table I compared with a reference sequence;
wherein the presence or absence of the at least one nucleotide sequence or variant thereof and/or the difference indicates that the microorganism is resistant to one or more antimicrobial drugs; and
wherein said antimicrobial drug capable of killing or inhibiting the growth of an antimicrobial drug resistant microorganism is different from the one or more antimicrobial drugs to which the microorganism is resistant.

45. The method of claim 44, wherein the microorganism is (1) an Acinetobacter species, (2) an Escherichia species, (3) a Klebsiella species, (4) a Morganella species, (5) a Proteus species, (6) a Pseudomonas species, (7) a Salmonella species, (8) a Serratia species, (9) a Stenotrophomonas species, (10) a Shigella species, (11) an Enterobacter species, (12) a Staphylococcus species, (13) a Burkholderia species, (14) a Citrobacter species, (15) a Kluyvera species, (16) a Pantoea species, or (17) a Raoultella species.

46. The method of claim 45, wherein prior to said administration, the sample containing or suspected of containing the antimicrobial drug resistant microorganism from the patient has been tested to determine:
(1) where the microorganism is an Acinetobacter species (i) the presence or absence in the Acinetobacter species of at least one nucleotide sequence identified in rows 0 to 183, 282 to 289, and 291 to 325 of Table I or a variant of the nucleotide sequence, and/or (ii) the presence in the Acinetobacter species of at least one difference in at least one nucleotide sequence identified in rows 184 to 281, 290, and 326 to 330 of Table I compared with a reference sequence;
(2) where the microorganism is an Escherichia species (i) the presence or absence in the Escherichia species of at least one nucleotide sequence identified in rows 1248 to 1499 of Table I or a variant of the nucleotide sequence, and/or (ii) the presence in the Escherichia species of at least one difference in at least one nucleotide sequence identified in rows 1500 to 1586 of Table I compared with a reference sequence;
(3) where the microorganism is a Klebsiella species (i) the presence or absence in the Klebsiella species of at least one nucleotide sequence identified in rows 1587 to 1696, 1743 to 1782, 1792 to 2133, 2208 to 2396, 2425 to 2427, and 2431 to 2446 of Table I or a variant of the nucleotide sequence, and/or (ii) the presence and/or absence in the Klebsiella species of at least one difference in at least one nucleotide sequence identified in rows 1697 to 1742, 1783 to 1791, 2134 to 2207, 2397 to 2424, 2428 to 2430, 2447, and 2448 of Table I compared with a reference sequence;
(4) where the microorganism is a Morganella species (i) the presence or absence in the Morganella species of at least one nucleotide sequence identified in rows 2460 to 2569 of Table I or a variant of the nucleotide sequence, and/or (ii) the presence and/or absence in the Morganella species of at least one difference in at least one nucleotide sequence identified in rows 2570 to 2621 of Table I compared with a reference sequence;
(5) where the microorganism is a Proteus species (i) the presence or absence in the Proteus species of at least one nucleotide sequence identified in rows 2623 to 2754, and 2789 to 2792 of Table I or a variant of the nucleotide sequence, and/or (ii) the presence and/or absence in the Proteus species of at least one difference in at least one nucleotide sequence identified in rows 2755 to 2788 of Table I compared with a reference sequence;
(6) where the microorganism is a Pseudomonas species (i) the presence or absence in the Pseudomonas species of at least one nucleotide sequence identified in rows 2793 to 2861, 2877, and 2878 of Table I or a variant of the nucleotide sequence, and/or (ii) the presence and/or absence in the Pseudomonas species of at least one difference in at least one nucleotide sequence identified in rows 2862 to 2876 of Table I compared with a reference sequence;
(7) where the microorganism is a Salmonella species (i) the presence or absence in the Salmonella species of at least one nucleotide sequence identified in rows 2885 to 2990 of Table I or a variant of the nucleotide sequence, and/or (ii) the presence in the Salmonella species of at least one difference in at least one nucleotide sequence identified in rows 2991 to 3008 of Table I compared with a reference sequence;
(8) where the microorganism is a Serratia species (i) the presence or absence in the Serratia species of at least one nucleotide sequence identified in rows 3009 to 3013, 3023 to 3028, 3031 to 3131, and 3188 to 3198 of Table I or a variant of the nucleotide sequence, and/or (ii) the presence and/or absence in the Serratia species of at least one difference in at least one nucleotide sequence identified in rows 3014 to 3022, 3029, 3030, 3132 to 3187, 3199, and 3200 of Table I compared with a reference sequence;
(9) where the microorganism is a Stenotrophomonas species (i) the presence in the Stenotrophomonas species of at least one nucleotide sequence identified in rows 3746 to 3751 of Table I or a variant of the nucleotide sequence;
(10) where the microorganism is a Shigella species (i) the presence or absence in the Shigella species of at least one nucleotide sequence identified in rows 3201 to 3234, 3237 to 3256, and 3263 to 3352 of Table I or a variant of the nucleotide sequence, and/or (ii) the presence in the Shigella species of at least one difference in at least one nucleotide sequence identified in rows 3235, 3236, 3257 to 3262, and 3353 to 3375 of Table I compared with a reference sequence;
(11) where the microorganism is an Enterobacter species (i) the presence or absence in the Enterobacter species of at least one nucleotide sequence identified in rows 692 to 697, 702 to 1074, 1190 to 1201, 1213 to 1226, 1231 to 1233, 1235 to 1239, 1246, and 1247 of Table I or a variant of the nucleotide sequence, and/or (ii) the presence and/or absence in the Enterobacter species of at least one difference in at least one nucleotide sequence identified in rows 698 to 701, 1075 to 1189, 1202 to 1212, 1227 to 1230, 1234, and 1240 to 1245 of Table I compared with a reference sequence;
(12) where the microorganism is a Staphylococcus species (i) the presence or absence in the Staphylococcus species of at least one nucleotide sequence identified in rows 3376 to 3664 of Table I or a variant of the nucleotide sequence, and/or (ii) the presence in the Staphylococcus species of at least one difference in at least one nucleotide sequence identified in rows 3665 to 3745 of Table I compared with a reference sequence;
(13) where the microorganism is a Burkholderia species (i) the presence or absence in the Burkholderia species of at least the nucleotide sequence identified in row 331 of Table I or a variant of the nucleotide sequence;
(14) where the microorganism is a Citrobacter species (i) the presence or absence in the Citrobacter species of at least one nucleotide sequence identified in rows 332 to 342, 345 to 361, 365 to 419, 422 to 581, 634 to 650, 653 to 683, 690, and 691 of Table I or a variant of the nucleotide sequence, and/or (ii) the presence in the Citrobacter species of at least one difference in at least one nucleotide sequence identified in rows 343, 344, 362 to 364, 420, 421, 582 to 633, 651, 652, and 684 to 689 of Table I compared with a reference sequence;
(15) where the microorganism is a Kluyvera species (i) the presence or absence in the Kluyvera species of at least one nucleotide sequence identified in rows 2449 to 2457 of Table I or a variant of the nucleotide sequence, and/or (ii) the presence in the Kluyvera species of at least one difference in at least one nucleotide sequence identified in rows 2458 to 2459 of Table I compared with a reference sequence;
(16) where the microorganism is a Pantoea species (i) the presence or absence in the Pantoea species of at least the nucleotide sequence identified in row 2622 of Table I or a variant of the nucleotide sequence; or
(17) where the microorganism is a Raoultella species (i) the presence or absence in the Raoultella species of at least one nucleotide sequence identified in rows 2879 to 2881, and 2883 of Table I or a variant of the nucleotide sequence, and/or (ii) the presence in the Raoultella species of at least one difference in at least one nucleotide sequence identified in rows 2882, and 2884 of Table I compared with a reference sequence.

47. The method of claim 46, wherein the difference in the nucleotide sequence is at position nc_pos of the nucleotide sequence identified in Table I.

48. The method of claim 47, wherein the difference at position nc_pos is the nc_alt residue for the nucleotide sequence identified in Table I.

49. The method of claim 46, wherein the difference in the nucleotide sequence is a point mutation resulting in a change in the encoded amino acid sequence.

50. The method of claim 46, wherein the one or more antimicrobial drugs to which the microorganism is resistant belong to a class of antimicrobial drugs selected from the group consisting of lactams; penicillins; quinolones and derivatives thereof; aminoglycosides; polyketides; benzene-derived compounds; sulfonamides; tetracyclines; cephalosporins; lincosamides; macrolides; nitrofuranes; glycopeptides; oxazolidinones; ansamycins; carbacephems; and folate synthesis inhibitors.

51. The method of claim 46, wherein the one or more antimicrobial drugs to which the microorganism is resistant is selected from the group consisting of amoxicillin/K clavulanate (AUG), ampicillin (AM), ampicillin/sulbactam (A/S), aztreonam (AZT), cefazolin (CFZ), cefepime (CPE), cefotaxime (CFT), ceftazidime (CAZ), ceftriaxone (CAX), cefuroxime (CRM), cephalotin (CF), ciprofloxacin (CP), ertapenem (ETP), gentamicin (GM), imipenem (IMP), levofloxacin (LVX), meropenem (MER), piperacillin/tazobactam (P/T), tetracycline (TE), tobramycin (TO), and trimethoprim/sulfamethoxazole (T/S).

52. A method for killing or inhibiting the growth of a population of antimicrobial resistant microorganisms, the method comprising the steps of:
(a) obtaining or providing a sample containing or suspected of containing at least one microorganism from a population of microorganisms;
(b) performing or having performed a genotyping assay on the sample to determine (i) the presence or absence in the microorganism of at least one nucleotide sequence identified in Table I or a variant of the nucleotide sequence and/or (ii) the presence or absence in the microorganism of at least one difference in at least one nucleotide sequence identified in Table I compared with a reference sequence; wherein the presence or absence of the at least one nucleotide sequence or variant thereof and/or the difference indicates that the microorganism is resistant to one or more antimicrobial drugs; and
(c) exposing the population of microorganisms identified as being resistant to an antimicrobial drug that is different from the one or more antimicrobial drugs to which the microorganism is resistant.

53. The method of claim 52, wherein the sample is obtained from a patient.

54. The method of claim 52, wherein the microorganism is (1) an Acinetobacter species, (2) an Escherichia species, (3) a Klebsiella species, (4) a Morganella species, (5) a Proteus species, (6) a Pseudomonas species, (7) a Salmonella species, (8) a Serratia species, (9) a Stenotrophomonas species, (10) a Shigella species, (11) an Enterobacter species, (12) a Staphylococcus species, (13) a Burkholderia species, (14) a Citrobacter species, (15) a Kluyvera species, (16) a Pantoea species, or (17) a Raoultella species; and
wherein step (b) comprises:
(b′ 1) performing or having performed a genotyping assay on the sample to determine (i) the presence or absence in the Acinetobacter species of at least one nucleotide sequence identified in rows 0 to 183, 282 to 289, and 291 to 325 of Table I or a variant of the nucleotide sequence, and/or (ii) the presence in the Acinetobacter species of at least one difference in at least one nucleotide sequence identified in rows 184 to 281, 290, and 326 to 330 of Table I compared with a reference sequence;
(b′2) performing or having performed a genotyping assay on the sample to determine (i) the presence or absence in the Escherichia species of at least one nucleotide sequence identified in rows 1248 to 1499 of Table I or a variant of the nucleotide sequence, and/or (ii) the presence in the Escherichia species of at least one difference in at least one nucleotide sequence identified in rows 1500 to 1586 of Table I compared with a reference sequence;
(b′3) performing or having performed a genotyping assay on the sample to determine (i) the presence or absence in the Klebsiella species of at least one nucleotide sequence identified in rows 1587 to 1696, 1743 to 1782, 1792 to 2133, 2208 to 2396, 2425 to 2427, and 2431 to 2446 of Table I or a variant of the nucleotide sequence, and/or (ii) the presence and/or absence in the Klebsiella species of at least one difference in at least one nucleotide sequence identified in rows 1697 to 1742, 1783 to 1791, 2134 to 2207, 2397 to 2424, 2428 to 2430, 2447, and 2448 of Table I compared with a reference sequence;
(b′4) performing or having performed a genotyping assay on the sample to determine (i) the presence or absence in the Morganella species of at least one nucleotide sequence identified in rows 2460 to 2569 of Table I or a variant of the nucleotide sequence, and/or (ii) the presence and/or absence in the Morganella species of at least one difference in at least one nucleotide sequence identified in rows 2570 to 2621 of Table I compared with a reference sequence;
(b′5) performing or having performed a genotyping assay on the sample to determine (i) the presence or absence in the Proteus species of at least one nucleotide sequence identified in rows 2623 to 2754, and 2789 to 2792 of Table I or a variant of the nucleotide sequence, and/or (ii) the presence and/or absence in the Proteus species of at least one difference in at least one nucleotide sequence identified in rows 2755 to 2788 of Table I compared with a reference sequence;
(b′6) performing or having performed a genotyping assay on the sample to determine (i) the presence or absence in the Pseudomonas species of at least one nucleotide sequence identified in rows 2793 to 2861, 2877, and 2878 of Table I or a variant of the nucleotide sequence, and/or (ii) the presence and/or absence in the Pseudomonas species of at least one difference in at least one nucleotide sequence identified in rows 2862 to 2876 of Table I compared with a reference sequence;
(b′7) performing or having performed a genotyping assay on the sample to determine (i) the presence or absence in the Salmonella species of at least one nucleotide sequence identified in rows 2885 to 2990 of Table I or a variant of the nucleotide sequence, and/or (ii) the presence in the Salmonella species of at least one difference in at least one nucleotide sequence identified in rows 2991 to 3008 of Table I compared with a reference sequence;
(b′8) performing or having performed a genotyping assay on the sample to determine (i) the presence or absence in the Serratia species of at least one nucleotide sequence identified in rows 3009 to 3013, 3023 to 3028, 3031 to 3131, and 3188 to 3198 of Table I or a variant of the nucleotide sequence, and/or (ii) the presence and/or absence in the Serratia species of at least one difference in at least one nucleotide sequence identified in rows 3014 to 3022, 3029, 3030, 3132 to 3187, 3199, and 3200 of Table I compared with a reference sequence;
(b′9) performing or having performed a genotyping assay on the sample to determine (i) the presence in the Stenotrophomonas species of at least one nucleotide sequence identified in rows 3746 to 3751 of Table I or a variant of the nucleotide sequence;
(b′10) performing or having performed a genotyping assay on the sample to determine (i) the presence or absence in the Shigella species of at least one nucleotide sequence identified in rows 3201 to 3234, 3237 to 3256, and 3263 to 3352 of Table I or a variant of the nucleotide sequence, and/or (ii) the presence in the Shigella species of at least one difference in at least one nucleotide sequence identified in rows 3235, 3236, 3257 to 3262, and 3353 to 3375 of Table I compared with a reference sequence;
(b′ 11) performing or having performed a genotyping assay on the sample to determine (i) the presence or absence in the Enterobacter species of at least one nucleotide sequence identified in rows 692 to 697, 702 to 1074, 1190 to 1201, 1213 to 1226, 1231 to 1233, 1235 to 1239, 1246, and 1247 of Table I or a variant of the nucleotide sequence, and/or (ii) the presence and/or absence in the Enterobacter species of at least one difference in at least one nucleotide sequence identified in rows 698 to 701, 1075 to 1189, 1202 to 1212, 1227 to 1230, 1234, and 1240 to 1245 of Table I compared with a reference sequence;
(b′12) performing or having performed a genotyping assay on the sample to determine (i) the presence or absence in the Staphylococcus species of at least one nucleotide sequence identified in rows 3376 to 3664 of Table I or a variant of the nucleotide sequence, and/or (ii) the presence in the Staphylococcus species of at least one difference in at least one nucleotide sequence identified in rows 3665 to 3745 of Table I compared with a reference sequence;
(b′13) performing or having performed a genotyping assay on the sample to determine (i) the presence or absence in the Burkholderia species of at least the nucleotide sequence identified in row 331 of Table I or a variant of the nucleotide sequence;
(b′14) performing or having performed a genotyping assay on the sample to determine (i) the presence or absence in the Citrobacter species of at least one nucleotide sequence identified in rows 332 to 342, 345 to 361, 365 to 419, 422 to 581, 634 to 650, 653 to 683, 690, and 691 of Table I or a variant of the nucleotide sequence, and/or (ii) the presence in the Citrobacter species of at least one difference in at least one nucleotide sequence identified in rows 343, 344, 362 to 364, 420, 421, 582 to 633, 651, 652, and 684 to 689 of Table I compared with a reference sequence;
(b′15) performing or having performed a genotyping assay on the sample to determine (i) the presence or absence in the Kluyvera species of at least one nucleotide sequence identified in rows 2449 to 2457 of Table I or a variant of the nucleotide sequence, and/or (ii) the presence in the Kluyvera species of at least one difference in at least one nucleotide sequence identified in rows 2458 to 2459 of Table I compared with a reference sequence;
(b′16) performing or having performed a genotyping assay on the sample to determine (i) the presence or absence in the Pantoea species of at least the nucleotide sequence identified in row 2622 of Table I or a variant of the nucleotide sequence; or
(b′17) performing or having performed a genotyping assay on the sample to determine (i) the presence or absence in the Raoultella species of at least one nucleotide sequence identified in rows 2879 to 2881, and 2883 of Table I or a variant of the nucleotide sequence, and/or (ii) the presence in the Raoultella species of at least one difference in at least one nucleotide sequence identified in rows 2882, and 2884 of Table I compared with a reference sequence.

55. The method of claim 54, wherein the genotyping assay comprises using a next generation sequencing or high throughput sequencing method.

56. The method of claim 54, wherein the difference in the nucleotide sequence is at position nc_pos of the nucleotide sequence identified in Table I.

57. The method of claim 56, wherein the difference at position nc_pos is the nc_alt residue for the nucleotide sequence identified in Table I.

58. The method of claim 54, wherein the difference in the nucleotide sequence is a point mutation resulting in a change in the encoded amino acid sequence.

59. The method of claim 54, wherein the one or more antimicrobial drugs to which the microorganism is resistant belong to a class of antimicrobial drugs selected from the group consisting of lactams; penicillins; quinolones and derivatives thereof; aminoglycosides; polyketides; benzene-derived compounds; sulfonamides; tetracyclines; cephalosporins; lincosamides; macrolides; nitrofuranes; glycopeptides; oxazolidinones; ansamycins; carbacephems; and folate synthesis inhibitors.

60. The method of claim 54, wherein the one or more antimicrobial drugs to which the microorganism is resistant is selected from the group consisting of amoxicillin/K clavulanate (AUG), ampicillin (AM), ampicillin/sulbactam (A/S), aztreonam (AZT), cefazolin (CFZ), cefepime (CPE), cefotaxime (CFT), ceftazidime (CAZ), ceftriaxone (CAX), cefuroxime (CRM), cephalotin (CF), ciprofloxacin (CP), ertapenem (ETP), gentamicin (GM), imipenem (IMP), levofloxacin (LVX), meropenem (MER), piperacillin/tazobactam (P/T), tetracycline (TE), tobramycin (TO), and trimethoprim/sulfamethoxazole (T/S).
